(19) United States
(12) Patent Application Publication
(10) Pub. No.: US 2025/0325690 A1
(43) Pub. Date: Oct. 23, 2025

(54) ANTIBODY-DRUG CONJUGATE WITH TWO TYPES OF DRUG-LINKER CONJUGATES ON SINGLE ANTIBODY

(71) Applicant: PINOTBIO, INC., Suwon-si, Gyeonggi-do (KR)

(72) Inventors: Doo Young JUNG, Suwon-si (KR); Jin Soo LEE, Suwon-si (KR); Hyun Yong CHO, Suwon-si (KR); Byeong Sung LEE, Suwon-si (KR); Seung Gun SHIN, Suwon-si (KR)

(73) Assignee: PINOTBIO, INC., Suwon-si, Gyeonggi-do (KR)

(21) Appl. No.: 18/857,043

(22) PCT Filed: Jun. 26, 2023
(86) PCT No.: PCT/KR2023/008865

§ 371 Date: Oct. 15, 2024

(30) Foreign Application Priority Data
Jun. 24, 2022 (KR) 10-2022-0077731
Dec. 7, 2022 (KR) 10-2022-0170146
Jan. 17, 2023 (KR) 10-2023-0006435

(51) Int. Cl. A61K 47/68; A61P 35/00; C07K 16/28; C07K 16/30; C07K 16/32

(52) U.S. Cl. CPC A61K 47/68037; A61K 47/68031; A61K 47/6845; A61K 47/6855; A61K 47/6889; A61P 35/00; C07K 16/2863; C07K 16/30; C07K 16/32

(57) ABSTRACT

Provided is an antibody-drug conjugate (ADC) having a drug-linker conjugate (A) linked thereto and a preparation method therefor, including a combination of a camptothecin-based drug that degrades DDX5 protein with a DAR of 4 or more and either (i) an acid-sensitive linker or (ii) an enzyme-sensitive linker. The antibody-drug conjugate is designed to increase the therapeutic index of the camptotechin-based drug payload while suppressing the non-selective uptake of the camptothecin-based drug and/or ADC released from apoptotic cells. The ADC is designed and/or synthesized so that two types of drug-linker conjugates including: a drug-linker conjugate (A) composed of a combination of a camptothecin-based drug that degrades DDX5 protein with a DAR of 4 or more and either (i) an acid-sensitive linker or (ii) an enzyme-sensitive linker; and a drug-linker conjugate (B) composed of a combination of a non-camptothecin cytotoxic drug and an enzyme-sensitive linker are each linked to a single antibody.

### CROSS REFERENCE TO RELATED APPLICATIONS

This application is a National Stage of International Application No. PCT/KR2023/008865 filed on Jun. 26, 2023, claiming priority based on Korean Patent Application No. 10-2022-0077731 filed on Jun. 24, 2022, Korean Patent Application No. 10-2022-0170146 filed on Dec. 7, 2022, and Korean Patent Application No. 10-2023-0006435 filed on Jan. 17, 2023, the entire disclosures of which are incorporated herein by references.

### TECHNICAL FIELD

The preset invention relates to an antibody-drug conjugate (ADC) in which a drug-linker conjugate (A) consisting of the combination of a camptothecin-based drug that degrades the DDX5 protein with a DAR of 4 or higher and (i) an acid-sensitive linker or (ii) an enzyme-sensitive linker is linked to one antibody, and which is designed to increase the therapeutic index of the ADC or its payload, which is a camptothecin-based drug, and inhibit the non-selective uptake of the camptothecin-based drug and/or ADC, which is released from apoptotic cells, and a method of preparing the same.

### BACKGROUND ART

Although immunotherapy is widely used in the treatment of cancer, traditional chemotherapy still plays an important role. Chemotherapeutic agents mainly use the difference in cell cycle to distinguish normal cells and cancer cells. To achieve a therapeutic effect, a chemotherapeutic agent is generally used at approximately the maximum tolerated dose. A chemotherapeutic agent targets cancer cells, but it only rapidly kills dividing cells without distinguishing between normal cells and cancer cells, making it difficult to avoid systemic toxicity and cytotoxicity. Therefore, there is a need for a method of specifically killing cancer cells by a cytotoxic drug, which is a chemotherapeutic agent, targeting only cancer cells.

A therapeutic monoclonal antibody specifically binds to an antigen present on the surface of a tumor cell, thereby causing a cell killing effect. It binds only to tumor cells, reducing the burden of non-specific systemic toxicity. Although therapeutic antibodies have advantages over chemotherapeutic agents, only a small number of antibodies among tumor-specific antibodies are used for cancer treatment. This is because many tumor-specific antibodies do not effectively kill cancer cells alone.

A therapeutic effect may increase when a chemotherapeutic agent with a strong killing capability is combined with an antibody with specificity for targeting only cancer cells. An antibody-drug conjugate (ADC) in which an antibody is combined with a chemotherapeutic agent was created with such expectation and hope.

An antibody has binding affinity and binding specificity for antigens. An ADC is a cytotoxic drug (payload) capable of destroying target cells, such as cancer cells, which is attached to an antibody that is accurately guided to a target. To prepare an ADC, three components, including these two components as well as a linker that conjugates these components, are needed.

ADCs have the potential to safely enhance the efficacy of a cytotoxic drug compared to when the drug is used alone. ADCs have a drug attached to an antibody to allow the antibody to specifically target a lesion and deliver the drug to the lesion, not normal tissue. In the case of cancer cells, an antibody specifically binding to a specific antigen expressed on the surface of a cancer cell is used to specifically deliver a drug with strong toxicity to the cancer cell to kill only the cancer cell.

ADCs must enter a target cell to work. The antibody of the ADC specifically binds to a specific antigen expressed on the surface of the target cell, such as a cancer cell, and then enters the target cell through the mechanism of action of the clathrin-coated pit of the cell membrane.

An ADC incorporated into the cell is separated from clathrin, fuses with another vesicle in the cell, and follows an endosome-lysosome pathway. Once reaching the endosome, the drug is separated from the antibody by specific elements in the endosome in the specific tumor cell environment. The free cytotoxic drug independent from the antibody enters the cytoplasm through the lysosomal membrane. The activated drug exerts a pharmacological effect by binding to its own molecular target in the vicinity, thereby inducing cell death and killing cancer cells.

Among these activities, some cytotoxic drugs may be passively diffused, actively transported, or released from cells via dead cells. When the drug that has spread to the surroundings penetrates the cell membrane, it enters an adjacent cell and causes a surrounding cell-killing effect that kills the surrounding cells as well (a so-called bystander phenomenon).

A significant number of cancer-specific antigens are expressed in a limited manner on the surface of cancer cells. In this case, it is not easy to deliver a sufficient amount of cytotoxic drug into cancer cells using ADCs, so alternatively, the strength of a toxin is increased. As the cytotoxic drug binding to an ADC, a drug stronger than general anticancer drugs was used.

So far, high toxicity and a narrow therapeutic window have been pointed out as problems, but recently, next-generation ADCs with reduced toxicity have emerged, and the ADCs have become the mainstream trend in the biopharmaceutical market.

ADCs are a rapidly growing class of anticancer therapeutics, with more than 100 ADCs in clinical trials. Currently, twelve ADCs such as gemtuzumab ozogamicin (Mylotarg), brentuximab vedotin (Adcetris), inotuzumab ozogamicin (Besponsa), trastuzumab emtansine (Kadcyla), polatuzumab vedotin (Polivy), enfortumab vedotin (Padcev), trastuzumab deruxtecan (Enhertu), sacituzumab govitecan (Trodelvy), belantamab mafodotin (Blenrep), loncastuximab tesirine (Zynlonta), tisotumab vedotin (Tivdak), and mirvetuximab soravtansine (Elahere) have been approved by the U.S. Food and Drug Administration. Further, a relatively small number of payload molecules (e.g., MMAE, MMAF, DM1, DM4, calicheamicin, SN38, Dxd, and PBD) are used in most approved and developmental ADCs.

Seattle Genetics developed Adcetris® by designing a linker that connects a dolastatin derivative, monomethyl auristatin E (MMAE), as a therapeutic agent to a cysteine residue of an anti-CD30 monoclonal antibody. The disulfide bonds of the anti-CD30 monoclonal antibody were partially reduced and then linked to a cleavage linker, such as a heterobifunctional maleimide linker (ValCit-PAB linker). The linker has a valine-citrulline peptide, which is sensitive to cathepsin B of a lysosome, which releases MMAE after internalization into CD30-positive cancer cells, killing target cancer cells.

Adcetris® was approved in 2011 for indications such as anaplastic large cell lymphoma and Hodgkin's lymphoma. MMAE released after cleaving a linker destroyed target cells and killed surrounding cancer cells after passing through the cell membrane, showing the effect of treating heterogeneous lymphoma.

Polivy®, developed by Genentech and Roche, is a second-generation cleavage linker that connects the anticancer drug MMAE to an anti-CD79b antibody. Polivy® was approved in 2019 as a combination therapy for diffuse large B-cell lymphoma. The average DAR value of Polivy® is 3.5 (Deeks, 2019).

Padcev®, developed by Seattle Genetics and Astellas, used the same linker, and approved in 2019 for patients with metastatic urothelial carcinoma who were previously treated with anti-PD1/PDL1 antibodies.

Recently approved new third-generation ADCs use improved cytotoxic drugs and novel linkers.

Trodelvy® developed by Immunomedics targeted a slightly overexpressed target, adopted a drug with less toxicity than the existing drugs such as MMAE or DM1, and allowed the drug to be released inside and outside a cell. Trodelvy®, approved by the US FDA in 2020, used a cleavable maleimide linker in which the topoisomerase I inhibitor SN-38 as a cytotoxic drug is attached to an anti-TROP2 monoclonal antibody with polyethylene glycol (PEG). Trodelvy® was approved for the indication of recurrent, refractory metastatic triple-negative breast cancer, which has been treated more than twice, which is an unmet medical need for which there is no existing therapeutic agent. The introduction of PEG to the linker increased the DAR to 7.6 (Goldenberg and Sharkey, 2020).

Daiichi Sankyo used DXD (exatecan), which is a cytotoxic drug that is about 10 times more active in cancer cells than SN-38, to develop Enhertu®. DXD is advantageous for the treatment of heterogeneous tumors due to having high solubility, relatively high safety, and a high bystander effect. However, it has a short half-life, which can reduce an off-target effect. DXD is bio-conjugated to the cysteine residue of an anti-HER2 antibody with a maleimide linker and has a homogeneous DAR value of 8. Despite the high DAR value, DXD is highly stable, with only a 2.1% release into the plasma over 21 days (Ogitani et al., 2016). Enhertu® was approved by the US FDA in 2019 for adult patients with unresectable metastatic Her2-positive breast cancer, who have received HER2-targeted therapy at least twice in the past.

GlaxoSmithKline (GSK) received approval for Blenrep®, a novel multiple myeloma ADC drug, in 2020. Blenrep® is an anti-B cell maturation antigen (BCMA, CD38) monocloncal antibody ADC and was approved as monotherapy for the treatment of recurrent or refractory multiple myeloma in adult patients, who have received at least four treatments, including a proteasome inhibitor, and an immunoregulator. Blenrep® is the first approved anti-BCMA therapeutic agent worldwide. Blenrep® has about four MMAF molecules, which are conjugated to the cysteine residue of an anti-BCMA monoclonal antibody using a non-cleavage protease-resistant maleimidocaproyl linker. Blenrep® eliminates cancer cells by various mechanisms, and in addition to the killing effect of MMAF, antibody-dependent cytotoxicity and antibody-dependent phagocytosis are involved.

The US biotechnology company, ADC Therapeutics, received approval for Zynlonta, which is an anti-CD19 monoclonal antibody ADC, in May 2021. Zynlonta was approved as a treatment for the indication of adult patients with relapsed or refractory (r/r) large B-cell lymphoma after two or more systemic therapies. This approval also includes diffuse large B-cell lymphoma, and diffuse large B-cell lymphoma arising from low-grade lymphoma and high-grade B-cell lymphoma, which were not otherwise specified. Zynlonta is the first to adopt a pyrrolobenzodiazepine (PBD) dimer as a cytotoxic drug. About 2.3 molecules of PBD dimers are linked to an antibody via a valine-alanine linker, which is cleaved by cathepsin B. When the drug is released, crosslinking between two strands is formed in a DNA groove, thereby killing target cells.

The potency of the cytotoxic drug (payload) binding to an ADC is typically 100 to 1000 times greater than that of the cytotoxic drug used alone. Therefore, it is necessary to develop ADCs that act highly specifically on target cancer cells without serious adverse effects in normal tissue.

The development of ADCs requires an understanding of the selection of a target antigen, the endocytosis of ADC by a tumor cell, drug potency, and the stability of a linker between the drug and the antibody. Moreover, the effects of ADC on drug binding ability according to the method of conjugating a cytotoxic drug with an antibody, a drug-antibody rate (DAR), antibody properties, and a linker type are very important for developing safe and effective ADCs.

The inhibitory factors of ADCs include a relatively difficult preparation process, a high cost, linker stability, and non-uniform DAR profiles.

Particularly, the stability of the linker that connects the antibody and the cytotoxic drug is closely related to toxicity in clinical trials.

That is, the biggest problem that can occur in ADCs is that when the linker connecting a monoclonal antibody and a cytotoxic drug in normal tissue, other than target cancer cells, is broken in an early stage, undesirable toxicity may occur. The reason that the first ADC, Mylotarg, in the US market was withdrawn in 2010 is known to be related to the instability of the linker attached to the antibody and side effects resulting from the strong toxicity of the cytotoxic drug. Another toxicity-related concern arises when an ADC targets an antigen that is also found in normal tissue

ADCs that had been approved to date and ADCs belonging to clinical pipelines are prepared by connecting small-molecule cytotoxic drugs to the lysine or cysteine residue of antibodies via linkers. In the conventional preparation processes for Adcetris and Kadcyla, and Mylotarg reapproved in 2017, the number of cytotoxic drugs attached to antibodies can be controlled in a limited manner. For example, according to the results of a study on Kadcyla's drug-antibody ratio (DAR) profile, four drugs are linked to an antibody on average, but there are about 80 available binding sites (lysine residues) on a monoclonal antibody, and the DAR profile varies from 0 to 8 because there are about 8 to 10 highly reactive lysine residues on the surface. Several companies are developing platforms for suitably conjugable drug-antibody ratios (DARs) including Besponsa (Inotuzumab ozogamicin) of Pfizer, newly approved in 2017.

However, ADCs including T-DM1 (trade name: Kadcyla®) have had problems of non-uniformity since their initial development. That is, since about 70 to 80 Lys residues on an antibody randomly react with a small-molecule drug, the DAR or conjugation sites are not uniform. It is known that when ADCs are produced by such a random conjugation method, the DAR ranges from 0 to 8, and multiple drugs with different numbers of drug conjugations are produced. Recently, it has been reported that when the number of drug conjugations and conjugation sites of ADCs are changed, the in vivo kinetics, drug release rate, and effects are changed. In this respect, the control over the number and site of conjugated drugs is required in next-generation ADCs. When the number and site are constant, the expected efficacy, variation of conjugated drugs, and lot difference, which are so-called regulation problems, can be solved.

As a method for antibody site-selective modification, there are a genetic engineering method and an enzyme-based modification method. Regarding the genetic engineering modification method, site selectivity and number selectivity can be controlled.

Recently, a chemical conjugation by affinity peptide (CCAP) method was developed (U.S. Ser. No. 10/227,383 B2, US 2021/0139548A1, ACS Omega (2019) Vol. 4, pp. 20564-20570, which are all incorporated herein by reference). The CCAP method is successful in the site-selective modification of an antibody by reacting a peptide reagent in which an NHS-activated ester and a drug are connected with affinity peptides with the antibody (i.e., a method of producing an ADC using a linker with a peptide moiety). The CCAP method is the first in the world to successfully site-selective modify an Fc region of an antibody by a chemical synthesis method, and has been confirmed to have satisfactory results in practice [reaction time: 30 min, yield: 70% (for DAR 1), site selectivity: 100%]. It has been demonstrated that the DAR can be controlled to 2 by adding about 5 equivalents of peptide reagent, and it is groundbreaking in that the modification site can also be controlled.

The drug-to-antibody ratio, DAR, is a very important characteristic that determines the pharmacodynamic properties and in vivo distribution in ADC development.

The higher the DAR, the higher the potency in in vitro experiments. However, contrary to expectations, ADCs with high DARs exerted low in vivo potency, this is presumed that the reason for this is the higher the number of drug conjugations, the higher the plasma clearance rate. Accordingly, the DAR of the ADC formulation has been regulated to about 2 to 4 for some time. For this reason, technologies that are used to conjugate a drug to the cysteine or lysine residues of an antibody are mainly used.

There are also problems due to hydrophobicity. Most of the commonly used cytotoxic drugs and linkers are hydrophobic. This causes problems such as ADC aggregation, the loss of affinity for a target antigen, or an increased plasma clearance rate. A hydrophilic linker containing sulfonate or polyethylene glycol (PEG) solves the problems caused by a hydrophobic linker. The PEG linker is water-soluble, and has low toxicity or low immunogenicity.

It has long been thought that a DAR of about 4 is optimal, which actually corresponds to second-generation linkers, which use MMAE or DM1 as a drug. For third-generation linkers, a higher DAR is better. Recently approved ADCs often have DAR values close to 8, and novel ADCs under clinical trials have varying DAR values ranging from 1 to 15.

Recently, a homogeneous ADC with a DAR value of 8 was produced by cysteine linkage, but this ADC also had an increased plasma clearance rate due to a unique drug-linker complex and great modifications to an antibody.

The concept of an “ideal” ADC is to deliver the maximum cytotoxic drug to target cancer cells. To develop high-DAR ADCs that remain in the plasma for a long time, a delicate balance between the number of drugs binding to an antibody and the degree of antibody modification must be studied and established.

Furthermore, as ADC technology develops, it is becoming more important to maximize anticancer efficacy by efficiently delivering payloads with various pharmacological effects or a combination thereof to cancer tissue rather than only selectively delivering a simply potent anticancer drug to cancer tissue using an antibody. Particularly, even when potent payloads such as MMAE, hemiasterlin, and PBD are used to prepare ADCs, unlike in preclinical animal tests, strong anticancer efficacy to the extent of completely eliminating cancer is not achieved in humans. Therefore, to improve the potency of ADCs, rather than simply using a large number of payloads such as MMAE, hemiasterlin, and PBD, a method of maximizing efficiency through combinations (e.g., a combination of an anti-apoptotic protein inhibitor (Bcl-XL inhibitor (navitoclax etc.) and an MMAE or Top 1 inhibitor, or a combination of a CHEK1 inhibitor and a Top 1 inhibitor) has been attempted, but to date, there is a problem that the method of efficiently preparing a dual payload ADC is limited. Current clinical attempts generally use a potent anticancer drug such as MMAE or Top 1 inhibitor in an ADC form, and a combination drug such as an anti-apoptotic protein inhibitor for systemic administration. While AbbVie is developing an ADC that uses a Bcl-XL inhibitor such as ABBV-155 as a payload, due to a limited number of antigens present on the surface of a cancer cell, there is a growing concern that the efficiency of drug delivery will be limited by the use of two types of ADCs, and it is predicted that its application will be limited due to the difficulty in using more than two types of ADCs, which are more expensive than general anticancer agents. Debiopharm delivers two types of drugs using a linker-payload system in which two types of payloads are combined in a 1:1 ratio, but there is still a limitation in that two types of drugs must be delivered only at a relatively high aggregation rate and a fixed ratio. In other words, there is still no commercialized ADC with a novel structure that can efficiently deliver more than two types of payloads at various ratios, while reducing the concern of aggregation.

As expected from the pharmacokinetics and biodistribution of monoclonal antibody drugs, in ADCs, high-affinity mAb binding to a cell membrane protein may localize a significant part of the mAb in a target cell population, and the chemical conjugation between a payload and anti-cancer mAb increases the selectivity of the payload being delivered to the cancer cells, thereby increasing the therapeutic index of the payload.

Although several ADCs have demonstrated sufficient efficacy and safety to receive FDA approval, all ADCs used in clinical practice cause significant toxicity in treated patients, and many ADCs fail during clinical development due to unacceptable toxicity profiles. This is because off-site toxicity remains problematic, limiting a tolerable ADC dose below that required for substantial anticancer efficacy. Even for FDA-approved ADCs, a considerable number of treated patients need adjuvant therapy to reduce the severity of ADC-related toxicity, and many patients require dose reduction, treatment delay, or treatment discontinuation.

An analysis of clinical data demonstrated that dose-limiting toxicities (DLTs) are often shared by multiple ADCs delivering the same cytotoxic payload, regardless of a target antigen and/or a cancer type to be treated. DLTs are typically associated with cells and tissue that do not express the target antigen (i.e., off-target toxicity), and often limit ADC doses below those required for optimal anticancer efficacy.

### DISCLOSURE

### Technical Problem

Recently, many ADCs have failed in clinical development due to excessive toxicity and unfavorable risk-benefit profiles, and even in the case of ADCs approved for clinical use, a considerable number of patients require dose reduction, treatment delay, or treatment discontinuation due to unacceptable ADC-related toxicity. To solve these problems, the present invention is directed to providing a modality for alleviating or preventing ADC toxicity.

In addition, in order to overcome the difficulties of ADC anticancer agents in that an increased anticancer effect increases toxicity, and conversely, the anticancer effect is not sufficiently exhibited when safety is increased, and thus the therapeutic window is narrowed, that is, to widen the therapeutic window of ADC drugs and increase a tumor response rate, and in order to solve the problem that the efficiency of drug delivery will be limited by using two types of ADCs because only a limited number of antigens are present on the surface of a cancer cell, and in order to provide a delicate balance between the number of drugs binding to the antibody and the degree of antibody modification, the present invention is directed to providing an antibody-drug conjugate (ADC) designed to bind two types of drug-linker conjugates, that is, a drug-linker conjugate (A) consisting of the combination of a camptothecin-based drug that degrades the DDX5 protein with a DAR of 4 or higher, and (i) an acid-sensitive linker or (ii) an enzyme-sensitive linker; and a drug-linker conjugate (B) formed by a combination of a non-camptothecin-based cytotoxic drug and an enzyme-sensitive linker, to one antibody.

### Technical Solution

A first aspect of the present invention provides a method of preparing an antibody-drug conjugate (ADC), in which a drug-linker conjugate (A) consisting of a combination of a camptothecin-based drug that degrades the DDX5 protein with a DAR of 4 or higher and (i) an acid-sensitive linker or (ii) an enzyme-sensitive linker is linked to one antibody, and which is designed to increase the therapeutic index of the ADC or its payload, which is a camptothecin-based drug, and inhibit the non-selective uptake of the camptothecin-based drug and/or ADC, which is released from apoptotic cells.

The method includes designing and/or synthesizing an ADC in which two types of drug-linker conjugates are connected such that a drug-linker conjugate (A) consisting of a combination of a camptothecin-based drug that degrades the DDX5 protein with a DAR of 4 or higher, and (i) an acid-sensitive linker or (ii) an enzyme-sensitive linker; and
a drug-linker conjugate (B) consisting of a combination of a non-camptothecin-based cytotoxic drug and an enzyme-sensitive linker each bind to one antibody.

A second aspect of the present invention provides an ADC in which two types of drug-linker conjugates are linked to one antibody,
wherein a drug-linker conjugate (A) consists of a combination of a camptothecin-based drug that degrades the DDX5 protein with a DAR of 4 or higher, and (i) an acid-sensitive linker or (ii) an enzyme-sensitive linker; and
a drug-linker conjugate (B-1) consists of a combination of a non-camptothecin super toxic drug having a DAR of 4 or less and an enzyme-sensitive linker or a drug-linker conjugate (B-2) consists of a combination of an anti-apoptotic protein inhibitor drug and an enzyme-sensitive linker each bind to one antibody.

The ADC in which two types of drug-linker conjugates are linked to one antibody may preferably have a total DAR of 6 to 10, and more preferably, about 8.

In the first and/or second aspect(s), by the additional connection of a drug-linker conjugate (B) consisting of the combination of a non-camptothecin-based cytotoxic drug and an enzyme-sensitive linker, non-selective uptake of the camptothecin-based drug-containing ADC in non-target cells may be inhibited.

In the first and/or second aspect(s), the non-camptothecin-based cytotoxic drug may alleviate or inhibit the adverse effects of the ADC by regulating the excessive bystander effect of the camptothecin-based drug released from both targeted/non-targeted apoptotic cells.

In the first and/or second aspect(s), the non-camptothecin-based cytotoxic drug may solve the problem of off-target toxicity of the camptothecin-based drug released from both targeted/non-targeted apoptotic cells.

A third aspect of the present invention provides a pharmaceutical composition for preventing or treating cancer, which includes the ADC of the second aspect or a pharmaceutically acceptable salt thereof as an active ingredient.

Here, the antibody may be trastuzumab, cetuximab, or sacituzumab.

Hereinafter, the present invention will be described.

In this specification, cancer and tumors may be used interchangeably.

The therapeutic index of a drug is a measure of the safety and efficacy of a drug in medical treatment. It is defined as the ratio between the dose of a drug that causes a therapeutic effect and the dose of a drug that exhibits toxicity or adverse effects. That is, it represents the range between the therapeutic dose and toxic dose of a drug.

A high therapeutic index indicates a wide margin of safety, where the effective dose is significantly lower than the toxic dose. This means that the drug can be administered at a therapeutic level without causing serious adverse effects or toxicity. Drugs with a high therapeutic index are generally considered safer and more preferable for clinical use.

On the other hand, low therapeutic indexes mean narrow safety margins. In these cases, the effective dose and toxic dose are relatively close, and the risk of adverse effects or toxicity when using a drug is high. Drugs with a low therapeutic index require careful monitoring and precise dosing to avoid harming patients.

The therapeutic index is an important consideration in drug development because it helps minimize the risk of adverse effects and determine the dose range that can provide a desired therapeutic effect. It provides useful information when prescribing medications and allows assessment of the overall benefit-to-risk ratio of the drug.

For a drug to act effectively in vivo, the drug concentration in the body must be maintained within the therapeutic range for a certain period of time. When the drug is present in an excessive amount in the body, it becomes toxic, and when the drug is present in an insufficient amount, it has no therapeutic effect.

Drug potency refers to the drug remaining in the body without degradation for the expected time, allowing it to exert an effect on a target indication. Since a lower metabolic rate leads to a blood concentration being maintained for a longer time, the duration of potency is prolonged.

In the specification, the term “antibody” refers to a protein molecule that acts as a ligand that specifically recognizes an antigen, including an immunoglobulin molecule that is immunologically reactive with a specific antigen, and includes all of a polyclonal antibody, a monoclonal antibody, and a whole antibody. The above term also includes a chimeric antibody, a bivalent or bispecific molecule, a diabody, a triabody, and a tetrabody. The antibody further includes single-chain antibodies (scABs) with a binding function to FcRn, derivatives of antibody constant regions, and artificial antibodies based on protein scaffolds. A whole antibody is a structure having two full-length light chains and two full-length heavy chains, wherein each light chain linked to a heavy chain by a disulfide bond. The whole antibodies include IgA, IgD, IgE, IgM, and IgG, wherein IgG is a subtype, including IgG1, IgG2, IgG3, and IgG4.

In the specification, the term “drug-linker conjugate” refers to a material for producing an ADC, in which the antibody is not yet connected.

In the specification, the “drug-linker conjugate is homogeneously and symmetrically connected” means that a drug/antibody ratio (DAR) and/or the conjugation site is/are constant.

In the specification, the camptothecin-based drug of Chemical Formula 1 or 2 may be provided by
(1) selecting an active camptothecin derivative designed to have a mechanism of action (MoA) that inhibits topoisomerase-1 and/or an MoA that degrades the oncoprotein DDX5 from the library of compounds having a camptothecin-based backbone represented by Chemical Formula 1 or 2 as a parent nucleus, and/or
(2) confirming whether the compound having a camptothecin-based backbone represented by Chemical Formula 1 or 2 as a parent nucleus inhibits the topoisomerase-1 and/or degrades the oncoprotein DDX5 through an in vitro experiment and/or an in vivo experiment.

A drug may be properly used by knowing the properties of the drug. Pharmacodynamic and pharmacokinetic parameters of the drug are helpful in understanding its properties.

Pharmacodynamics explains the magnitude and patterns of changes (effects; cell viability, a clinical effect, a therapeutic action, a toxic effect, and an adverse effect), which occur in cells or the body after binding a drug to a receptor, in relation to drug concentration.

Pharmacokinetics (PK) shows how a drug concentration changes as a drug moves through different compartments of the body through ADME depending on the drug or drug modality.

Meanwhile, the in vivo efficacy and in vivo side effects of an anticancer agent are closely related to the absorption, distribution, metabolism, and excretion (ADME) properties of the anticancer agent. The ADME of a drug determines pharmacokinetics, which describes how the drug moves through the body and interacts with tissue and organs.

ADCs can be used as a type of targeted therapy in cancer treatment. For example, a drug is specifically delivered to cancer cells by combining a monoclonal antibody with a cytotoxic drug. The monoclonal antibody component of the ADC recognizes and binds to a specific antigen present on cancer cells, whereas the cytotoxic drug component kills cancer cells when internalized.

Additionally, an ADC is a novel anticancer therapeutic modality that binds an anticancer drug exhibiting a potent anticancer effect at a low concentration of several pM to several nM to an antibody that can selectively recognize cancer cells by binding to a specific antigen expressed on the surface of the cancer cell using a linker that maintains binding without releasing the drug until the ADC is internalized inside the cell and then releases the drug immediately after being internalized in the cells.

Although ADCs have shown new potential in combining antibody selectivity and the potent cell toxicity of an anticancer drug to provide powerful anticancer therapeutic efficacy while reducing the risk of systemic side effects in many cancer patients, many existing ADCs show several limitations in practical application. When a DAR exceeds a certain number, due to the hydrophobicity of the drug and linker used, an ADC has problems of indiscriminate absorption (non-selective uptake) into normal cells/normal tissue, other than cancer cells, low PK profiles caused by drug release, and unexpected toxicity.

In addition, the potency is limited due to the limitation in the amount of drug delivered.

Generally, when a mostly hydrophobic linker-payload is bound to a hydrophilic antibody, the DAR, which indicates the number of drugs attached to one antibody, does not have a big problem in the range of 1 to 8, but when this range is exceeded, a problem may occur in production/transportation/use due to aggregation or in safety due to the formation of an aggregate in blood. In addition, due to high lipid solubility, non-selective uptake may occur in macrophages, rather than drug absorption and payload release caused by drug targeting, resulting in unexpected adverse effects. In addition, an antibody (ADC) dose may be increased while fixing the DAR at a certain value. However in this case, due to (1) the problem of non-selective uptake of ADCs and subsequent off-target toxicity and (2) the problem in that when the antibodies constituting the ADCs bind to the drug surface on the surface of a cancer cell with high affinity, the drug target around a blood vessel is first saturated, and after more than a certain amount of antibodies are bound, an additional antibody (ADC) does not penetrate into cancer tissue, resulting in a problem of the administered ADC becoming useless.

Among these problems, first, to solve the problem of off-target toxicity occurring after non-selective uptake, many ADCs using a camptothecin compound exhibiting relatively high safety in normal tissue are being developed, and particularly, the success of Enhertu (Daiichi-Sankyo) and Trodelvy (Gilead) shows that this approach is effective. That is, a certain part of the excellent success of Enhertu is achieved by increasing the ADC dose from a convention 2.7 mpk level to 4.8-5.4 mpk by using a safe payload, and thereby evenly distributing the payload released from the ADC throughout the cancer tissue.
in the case of Enhertu (Daiichi-Sankyo), which is a high DAR ADC using the combination of a payload with a relatively low toxicity and a hydrophilic linker, to conjugate the drug and the linker to an antibody, a pseudo-homogeneous ADC with a DAR of approximately 8 was obtained by reacting all eight free thiol functional groups, which are obtained after cleaving all interchain disulfide bonds of an IgG1 format, with a drug-linker conjugate. Here, the linker and payload used have stronger hydrophilicity than previously used linkers and payloads, so they rarely experience aggregation even at a high DAR value, and a stable PK profile can also be maintained. Nevertheless, the problem of non-selective uptake is still present, which results in severe inflammatory side effects in 10 to 15% of patients.

In order to provide an ADC designed to increase the therapeutic index of an antibody-drug conjugate (ADC) in which a drug-linker conjugate (A) consisting of t a combination of a camptothecin-based drug degrading the DDX5 protein with a DAR of 4 or higher and (i) an acid-sensitive linker or (ii) an enzyme-sensitive linker is conjugated to one antibody or its payload, such as a camptothecin-based drug, and to inhibit the non-selective uptake of the camptothecin-based drug and/or released from apoptotic cells,
the present invention is characterized by designing and/or synthesizing an ADC in which two types of drug-linker conjugates are linked such that a drug-linker conjugate (A) consisting of the combination of a camptothecin-based drug that degrades the DDX5 protein with a DAR=4 or higher, and (i) an acid-sensitive linker or (ii) an enzyme-sensitive linker; and a drug-linker conjugate (B) consisting of the combination of a non-camptothecin-based cytotoxic drug and an enzyme-sensitive linker are each linked to one antibody.

The term “non-selective uptake” refers to the general absorption of a material into cells without a specific target or preference. In the context of ADCs, non-selective uptake refers to the absorption of ADC or its payload into cells not expressing a target antigen.

It is estimated that only about 0.1% of the injected ADC dose is delivered to the targeted disease cell population, and the majority of the administered dose may potentially cause undesired toxicity by being metabolized “off-site” in non-target healthy cells. The off-site ADC toxicity may be classified into “on-target” or “off-target,” and on-target toxicity occurs through ADC binding to a target cell surface protein on healthy cells. Each component of the ADC including an antibody, a linker, and a payload may affect the degree of toxicity caused by the ADC.

The mechanism of ADC toxicity is illustrated in FIG. 5. The uptake of an intact ADC into normal cells may occur via non-specific intracellular uptake or via internalization by binding to a target antigen or an Fc/C type lectin receptor. In the case of payloads released by ADC deconjugation in the extracellular fluid or from other targeted/non-targeted apoptotic cells, membrane-permeable payloads may enter normal cells through passive diffusion, or non-membrane-permeable linker-payload adjuncts may enter normal cells through non-specific endocytosis.

Conceptually, ADCs can enhance the selectivity of chemotherapy by facilitating targeted delivery of a cytotoxic payload molecule to a desired cell population (on-target, on-site toxicity), and also broaden a therapeutic index by reducing payload delivery to non-target healthy tissue.

In the early stage of technology development, the anticipated safety concern for anticancer ADCs was on-target (i.e., target-mediated) toxicity in tissue where a target antigen is expressed to some extent, and the differential expression of targets in cancer cells and healthy tissue is expected to be an important factor in determining the therapeutic index of ADCs. However, subsequent clinical experience with ADCs demonstrated that dose-limiting toxicity (DLT) is rarely caused by target expression in healthy tissue.

According to preclinical and clinical data in twenty investigational new drug (IND) applications for ADCs, submitted from 2012 to 2013, ADCs having the same type of linker/payload generally share very similar toxicity profiles, DLTs, and maximum tolerated doses (MTDs), regardless of a target antigen and the degree of expression of the antigen in healthy tissue.

Since lipophilic payloads have high plasma membrane permeability, the released payloads efficiently enter non-target cells (e.g., through membrane diffusion), potentially causing undesired cytotoxicity.

As a method for reducing the non-selective uptake of ADCs, lowering the DAR is already a well-established method, but for an ADC using a drug with relatively low cytotoxicity such as a camptothecin-based payload, it is also important to maintain a high-DAR of 4 or more to ensure sufficient potency.

In MDA-MB-468, which is a Her2 negative cell line corresponding to “non-selective uptake” in ADCs, the IC50 result for trastuzumab-25-6 (DAR 6; IC50=97.61 nM) was 2.5 times lower than that for trastuzumab-25-6 (DAR 4; IC50=240.7 nM).

This may increase the therapeutic index in terms of off-target toxicity (by reducing payload delivery to non-target healthy tissue) by regulating the DAR of a camptothecin-based payload. That is, in the case of DAR=4, compared to the case of DAR=6, even when a higher content of ADC is administered, the bystander effect, which is a type of “non-selective uptake” of a payload, may be maintained lower.

As shown in FIG. 1, the evaluation results for trastuzumab-MMAE(2)-25-6(6) showed that, in the MDA-MB-453 cell line, which is a Her2 positive cell line,
(1) the dual payload ADC has higher potency (lower IC50) than single payload ADCs, and (2) the dual payload ADC has a similar level of potency in an in vitro experiment to the combination of single payload ADCs (trastuzumab-25-6 (DAR 6)+trastuzumab-MMAE (DAR 2)).

However, it was confirmed that, in MDA-MB-468, which is a Her2 negative cell line, (3) the dual payload ADC has high stability by showing a significantly higher IC50 value than the combination of single payload ADCs (in terms of the off-target toxicity of ADC). A relatively equivalent or higher level of stability, compared to ˜

As shown in FIG. 2, the evaluation results for trastuzumab-veliparib(4)-25-6(4) showed that, in the MDA-MB-453 cell line, which is a Her2 positive cell line,
(1) the dual payload ADC has higher potency (lower IC50) than single payload ADCs, and (2) the dual payload ADC has higher potency (lower IC50) than the combination of single payload ADCs (trastuzumab-veliparib (DAR 4)+trastuzumab-25-6 (DAR 4)).

However, it was confirmed that, in the MDA-MB-468 cell line, which is a Her2 negative cell line, (3) the dual payload ADC has relatively the same or higher stability, compared to the combination of single payload ADCs (trastuzumab-veliparib (DAR 4)+trastuzumab-25-6 (DAR 4)).

In summary, it was confirmed that (1) the dual payload ADC has excellent potency compared to single ADCs with the same DAR, and (2) in the case of trastuzumab-MMAE(2)-25-6(6), the dual payload ADC has the same level of potency but relatively higher stability, compared to a combination of two types of single ADCs with the same DAR, and (3) in the case of trastuzumab-veliparib(4)-25-6(4), the dual payload ADC has the same level of stability but a relatively higher potency, compared to a combination of two types of single ADCs with the same DAR.

Based on the analysis of the above-described results in FIGS. 1 and 2, the present invention provides an ADC in which a drug-linker conjugate (A) consisting of a combination of a camptothecin-based drug that degrades the DDX5 protein with a DAR=4 or higher and (i) an acid-sensitive linker or (ii) an enzyme-sensitive linker binds to one antibody, and which is designed to increase the therapeutic index of the ADC or its payload, which is a camptothecin-based drug, and inhibit the non-selective uptake of the camptothecin-based drug and/or ADC, which is released from apoptotic cells, thereby completing the present invention.

[Clinical Results and Adverse Effects of ADCs with Camptothecin-Based Payloads]

Trastuzumab deruxtecan (Enhertu) is a humanized anti-HER2 antibody linked to the camptothecin derivative deruxtecan using a stable linker. In a first phase 1 dose-escalation study, 22 patients with HER-2-positive advanced or metastatic breast cancer, gastric cancer, or other HER-2-expressing solid tumors were treated with 0.8 mg/kg to 8.0 mg/kg of trastuzumab deruxtecan once every three weeks. No dose-limiting toxicity was observed and the MTD was not reached. Target drug exposure was achieved at a dose of 6.4 mg/kg, which is selected as the recommended phase 2 dose.

The pivotal single-group, phase 2 clinical trial, DESTINY-Breast01 clinical trial, consisted of two parts. In the first part, advanced/metastatic breast cancer patients who had previously received more than two anti-HER2 regimens were randomly assigned to receive trastuzumab deruxtecan at a dose of 5.4 mg/kg (n=50), 6.4 mg/kg (n=48), or 7.4 mg/kg (n=21) once every three weeks. In the second part, based on the efficacy and toxicity data obtained in Part 1, trastuzumab deruxtecan was administered to 134 patients at a dose of 5.4 mg/kg. Among the 184 patients that had been treated with 5.4 mg/kg of trastuzumab deruxtecan, the most common adverse reactions across all grades (≥20%) were nausea (77.5%), fatigue (49.8%), hair loss (49.8%), vomiting (44.3%), neutropenia (40.3%), constipation (37.5%), anemia (33.6%), decreased appetite (33.2%), diarrhea (29.2%), leukopenia (26.9%), and thrombocytopenia (24.9%). Grade 3 or higher adverse reactions occurred in 57.1% of patients, with the most common being neutropenia (20.7%), anemia (8.7%), nausea (7.6%), leukopenia (6.5%), lymphopenia (6.5%), and fatigue (6.0%). Drug discontinuation, dose reduction, and treatment discontinuation, caused by adverse reactions, occurred in 35.3%, 23.4%, and 15.2% of patients, respectively, with pneumonia (11 patients) and interstitial lung disease (5 patients) being the most common reasons. The black box warning for patients treated with trastuzumab deruxtecan includes interstitial lung disease (ILD) and pneumonia. Treatment-related interstitial lung disease and fatal results occurred in 9% and 2.6% of patients treated with trastuzumab deruxtecan, respectively. As with other HER-2-targeted ADCs, patients treated with trastuzumab deruxtecan had an increased risk of embryo-fetal toxicity and left ventricular dysfunction.

Meanwhile, sacituzumab govitecan (Trodelvy) is humanized anti-TOP-2 IgG linked to an active metabolite of irinotecan (SN-38) via a pH-sensitive linker sensitive. In a first-in-human, dose-escalation phase 1/2 clinical trial, 25 patients with a variety of metastatic solid tumors were administered sacituzumab govitecan at 8 mg/kg to 18 mg/kg on day 1 and day 8 in a 21-day cycle. The MTD for the first cycle was determined to be 12 mg/kg, with neutropenia as the dose-limiting toxicity. However, this dose level was too toxic for subsequent cycles, so doses of 8 mg/kg and 10 mg/kg were selected for the phase-2 clinical trial. In the phase-2 clinical trial, sacituzumab govitecan was administered at a dose of 8 mg/kg (n=81) or 10 mg/kg (n=97) to patients with various types of metastatic epithelial cancer who had previously received multiple treatments. The most common adverse reactions across all grades (≥25%) reported in the 8 mg/kg and 10 mg/kg cohorts were nausea (59% vs. 63%), diarrhea (53% vs. 62%), neutropenia (42% vs. 58%), fatigue (61% vs. 52%), vomiting (36% vs. 43%), anemia (38% vs. 42%), hair loss (46% vs. 37%), and constipation (33% vs. 37%). The most common grade 3 or higher adverse reactions (≥10%) reported in the 8 mg/kg and 10 mg/kg-administered groups were neutropenia (30% vs. 36%), anemia (13% vs. 12%), diarrhea (4% vs. 10%), and leukopenia (6% vs. 12%). Dose reductions occurred in 19% and 28% of patients in the 8 mg/kg and 10 mg/kg cohorts, respectively. Neutropenia was the most common adverse reaction leading to dose delay or reduction. After the first administration, significantly more patients in the 10 mg/kg cohort experienced grade 3 or higher neutropenia than in the 8 mg/kg cohort (47% vs. 21%).

A black box warning for severe or life-threatening neutropenia and severe diarrhea has been added to the sacituzumab govitecan label. Such an adverse reaction is more likely to be mediated by released (“free”)SN-38, and associated with the same toxicity as the SN-38 prodrug irinotecan. Among all patients treated with sacituzumab govitecan, neutropenia of all grades and grade 3 or higher occurred in 61% and 47%, respectively. 7% of patients had febrile neutropenia. Diarrhea of all grades and grade 3 or higher occurred in 65% and 12% of all patients treated with sacituzumab govitecan, respectively. Neutropenic colitis occurred in 0.5% of patients.

### [Anticancer Mechanism of FL118 Drug as Molecular Glue Degrader Binding to DDX5]

FL118 may serve as a molecular glue degrader that degrades DDX5 by directly binding DDX5 to a ubiquitination regulator.

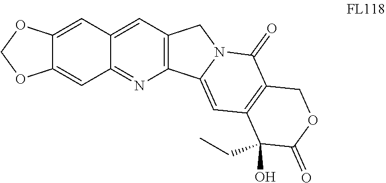 [SMILES: CC[C@@]1(O)C(=O)OCc2c1cc1n(c2=O)Cc2cc3cc4c(cc3nc2-1)OCO4]

FL118, acting as a molecular glue, directly binds to the oncoprotein DDX5, which is a multifunctional master regulator, and has a function of dephosphorylating and degrading DDX5 through a proteasome degradation pathway without reducing DDX5 mRNA, and DDX5 silencing indicates that DDX5 is a master regulator that regulates the expression of several oncogenic proteins, including survivin, Mcl-1, XIAP, cIAP2, c-Myc, and mutant Kras.

In addition, FL118 can avoid resistance by fundamentally blocking the overexpression of an anti-apoptotic protein, which is a common mechanism of resistance development in most types of cancer; and has biomarkers (DDX5, K-ras, and p53) capable of predicting anticancer responses in patients, and companion diagnostic techniques, which have been already established, enables personalized treatment through companion diagnostics by ensuring customized biomarkers, and exhibits strong efficacy, particularly, in p53/K-ras mutant cancer cells with poor prognosis.

FL118 indirectly controls DDX5 downstream targets to suppress cancer initiation, development, metastasis, recurrence and treatment resistance with high efficacy as proven in studies using human colorectal cancer/pancreatic ductal adenocarcinoma cells, and tumor models.

The genetic manipulation of DDX5 in PDAC cells affects tumor growth. PDAC cells with DDX5 KO are resistant to FL118 treatment. In studies with human tumor animal models, FL118 exerts high efficacy to remove human PDAC and CRC tumors with high DDX5 expression, but FL118 is proven to be less effective in PDAC and CRC tumors with low DDX5 expression.

The DDX5 protein is a direct target of the FL118 drug and may serve as a biomarker for predicting PDAC and CRC tumor sensitivity to FL118.

Meanwhile, the FL118 drug has a Top1 inhibitory effect equivalent to or higher than SN-38 in cancer cells, and exhibits cytotoxicity in various cancer lines with a low IC₅₀ value (FIG. 18), which is 5 to 20 times higher than SN-38, and the results of evaluation on 140 cell lines originating from various cancer types also showed very strong anticancer efficacy, with IC₅₀ of <100 nM against most cancer cells. The FL118 drug exhibited excellent safety through GLP-toxicity tests in mice and beagle dogs, and showed superior efficiency compared to SN-38 in various cancer cell line xenograft models. Meanwhile, when used for patients, the camptothecin-based anticancer agent initially showed excellent anticancer responses, but showed strong tolerance to the drugs through the epigenetic silencing of the Top1 gene and the Top2 dependence of cancer cells. In contrast, the FL118 drug showed strong efficacy even in a xenograft model in which Top1 is not expressed through epigenetic silencing or knock-out.

In addition, the FL118 drug is a triple-target anticancer agent that inhibits resistance proteins involved in the resistance mechanism, such as the Bcl family including survivin, and simultaneously inhibits the action of an efflux pump, while directly targeting topoisomerase-1, which is a well-established anticancer target.

Specifically, while camptothecin-based anticancer agents such as SN-38 show resistance in the form of drug excretion from cells by the overexpression of the ABCG2 transporter, the FL118 drug is not affected by the ABCG2 transporter, so it is possible to overcome the resistance caused thereby. The FL118 drug strongly suppresses the expression of an anti-apoptotic protein (e.g., survivin, cIAP2, XIAP, etc.), which is another main cause of resistance to anticancer agents, at a low concentration, thereby blocking the expression of resistance (FIG. 17).

Accordingly, the FL118 drug is not released out of cells by the efflux pump ABCG2, and is able to block the resistance caused by various anti-apoptotic proteins. As a result, the FL118 drug may overcome various resistance action mechanisms of SN-38/exatecan.

The FL118 drug showed strong tumor regression efficacy compared to SN-38 in colorectal cancer/head and neck cancer/pancreatic cancer when administered in vivo at the same amount as SN-38.

The FL118 drug exhibited potent anticancer efficacy when FL118 was administered after inducing SN-38 resistance in tumor xenografts.

Moreover, the FL118 drug has PK/stability profiles, which are optimal for applying targeted drug delivery (e.g., carrier-drug conjugate). When the FL118 drug is systemically administered alone, it is rapidly metabolized/excreted from the blood and only shows a low concentration, but it accumulates rapidly in cancer tissue immediately after administration and maintains a high concentration for a long time. For example, the maximum selectivity between tumor tissue and normal tissue is ensured when applying ADCs.

Based on this, in order to exhibit various advantages as an anticancer agent exemplified by the FL118 drug, various camptothecin-based drugs of Chemical Formula 1 or Chemical Formula 2 according to the present invention may be designed and used as payloads.

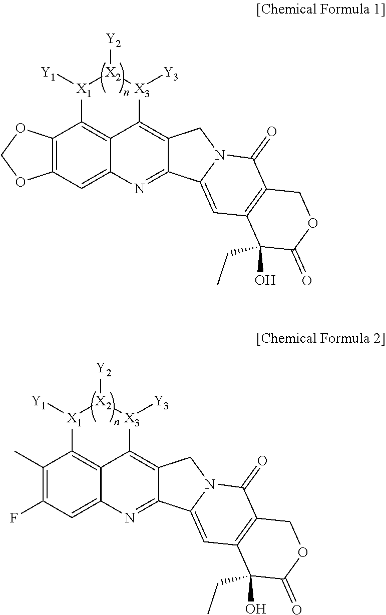

here, X₁ and X₃ may each be independently carbon, oxygen, nitrogen, or sulfur, and may be the same or different,
X₂ may be carbon, oxygen, nitrogen, sulfur, a single bond, or a double bond,
X₁, (X₂)ₙ and X₃ may form a 5-, 6- or 7-membered ring (n=0 to 2), and
Y₁, Y₂ and Y₃ may each be independently hydrogen, or a functional group containing oxygen, nitrogen, phosphorus or sulfur.

Here, non-limiting examples of the functional groups including oxygen, nitrogen, phosphorous, or sulfur may include functional groups selected from the group consisting of —CHO, —COOH, —NH₂, —SH, —CONH₂, —PO₃H, —PO₄H₂, —OPO₄H, —PO₂(OR¹)(OR²)(R¹, R²═CₛHₜNᵤO_{w}SₓP_{y}X_{z}, X═—F, —Cl, —Br or —I, 0≤s≤20, 0≤t≤2(s+u)+1, 0≤u≤2s, 0≤w≤2s, 0≤x≤2s, 0≤y≤2s, 0≤z≤2s), —SO₃H, —OSO₃H, —NO₂, —N₃, —NR₃OH(R═CₙH₂ₙ₊₁, 0≤n≤16), —NR₃⁺X⁻(R═CₙHₘ, 0≤n≤16, 0≤m≤34, X═OH, Cl or Br), NR₄⁺X⁻(R═CₙHₘ, 0≤n≤16, 0≤m≤34, X═OH, Cl or Br), —COSH, —COOCO—, —CORCO— (R═CₗHₘ, 0≤l≤3, 0≤m≤2l+1), —COOR, —CN, —N₃, —N₂, —NROH(R═CₛHₜNᵤO_{w}SₓP_{y}X_{z}, X═—F, —Cl, —Br or —I, 0≤s≤20, 0≤t≤2(s+u)+1, 0≤u≤2s, 0≤w≤2s, 0≤x≤2s, 0≤y≤2s, 0≤z≤2s), —NR¹NR²R³ (R¹, R², R³═CₛHₜNᵤO_{w}SₓP_{y}X_{z}, X═—F, —Cl, —Br or —I, 0≤s≤20, 0≤t≤2(s+u)+1, 0≤u≤2s, 0≤w≤2s, 0≤x≤2s, 0≤y≤2s, 0≤z≤2s), —CONHNR¹R² (R¹, R²═CₛHₜNᵤO_{w}SₓP_{y}X_{z}, X═—F, —Cl, —Br or —I, 0≤s≤20, 0≤t≤2(s+u)+1, 0≤u≤2s, 0≤w≤2s, 0≤x≤2s, 0≤y≤2s, 0≤z≤2s), —NR¹R²R³X′ (R¹, R², R³═CₛHₜNᵤO_{w}SₓP_{y}X_{z}, X═—F, —Cl, —Br or —I, X′═F—, Cl—, Br—, or I—, 0≤s≤20, 0≤t≤2(s+u)+1, 0≤u≤2s, 0≤w≤2s, 0≤x≤2s, 0≤y≤2s, 0≤z≤2s), —OH, —O—, >C═O, —SS—, —SO—, —NO₂, —COX (X═F, Cl, Br or I), —COOCO—, —CONH—, —CN, —SCOCH₃, —SCN, —NCS, —NCO, —OCN, —CN, —F, —Cl, —I, —Br, epoxy, -hydrazone, —ONO₂, —PO(OH)₂, —C═NNH₂, —HC═CH—, —C═C—, —C≡C—, and a hydrocarbon with two or more carbon atoms.

Exatecan is a camptothecin derivative, which is an anti-tumor small molecule compound that inhibits topoisomerase-1. Exatecan is a material with cytotoxicity that is 5 to 10 times more potent than the SN-38 drug.

Exatecan differs from irinotecan and does not require enzymatic activation. In addition, it has stronger topoisomerase-1 inhibitory activity than SN-38, which is the active ingredient of irinotecan, or topotecan used in the same clinical trial, and stronger cytotoxic activity against various cancer cells in vitro. Particularly, it also showed effectiveness against cancer cells that exhibit a tolerance to SN-38 or the like by the expression of P-glycoprotein. In addition, it showed a strong anti-tumor effect in a human tumor subcutaneously transplanted mouse model, and clinical trials were conducted.

The exatecan derivative for an ADC (Dxd) is a strong DNA topoisomerase-1 inhibitor, which is used as a HER2-targeting ADC (DS-8201a) conjugation drug, and has an IC₅₀ of 0.31 μM.

Surprisingly, as will be described below, while designing camptothecin derivatives having a structure exhibiting a dual MoA in terms of topoisomerase-1 inhibition and DDX5 degradation, which are the advantages of the FL118 drug, based on the structure of the FL118 drug differentiated from the SN38 drug (FIGS. 3 and 4), the present inventors found that exatecan or Dxd also exhibits a MoA that degrades the DDX5 protein (FIGS. 26 and 27).

The camptothecin-based drugs of Chemical Formula 1 or Chemical Formula 2, which are used as payloads in the present invention, are designed so that R₁ and R₂ (Group A) on ring A in General Formula 1 are the same or structurally very similar to the FL118 compound or exatecan/Dxd (FIG. 3), thereby exhibiting an anticancer mechanism that degrades the oncoprotein DDX5 in cells (Example 1).

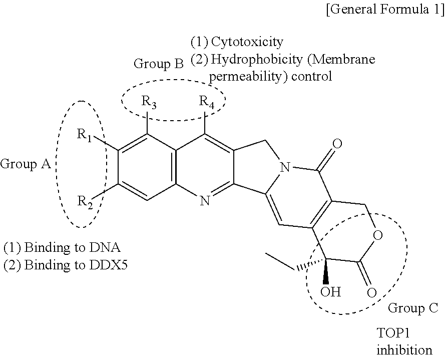

The synthetic design concept of the active camptothecin derivative having a dual MoA that degrades the oncoprotein DDX5 as well as the ability to inhibit topoisomerase-1 may be to design a structure that can maintain the structure of Group C, which is a topoisomerase-1 inhibition region based on the structure activity relationship (SAR), and the same structure of Group A, which is a binding site for DDX5 degradation, as FL118, or the same structure as exadecan and Dxd, improve the physicochemical properties of the drug by modifying the structures (R₃ and R₄) of Group B in General Formula 1 as shown in Chemical Formula 1 or Chemical Formula 2 to solve the aggregation of ADCs using it as a payload, and precisely control a bystander effect by regulating the cytotoxicity of a payload (active camptothecin-based drug) released from an ADC, and/or tumor tissue penetration and/or cell membrane permeability of the drug.

In General Formula 1, Group C may bind to topoisomerase-1 and Group A may bind to DNA, such that the covalent bonding of the topoisomerase-DNA complex may be stabilized, preventing the re-ligation of cleaved DNA fragments. Alternatively, in General Formula 1, Group A may bind to DDX5 and Group C may bind to an E3 ligase, thereby inducing DDX5 degradation (refer to PCT/KR2023/005380, which is incorporated herein in its entirety).

It is preferable to design the camptothecin-based drugs of Chemical Formula 1 or Chemical Formula 2 to exhibit a suitable bystander effect in tumor tissue by regulating the cell membrane permeability through the modification of R₃ and/or R₄ in General Formula 1.

Accordingly, considering various adverse effects as well as problems such as a reduced therapeutic coefficient, another key feature of the present invention is that the selection range of an ADC payload exhibiting appropriate anticancer efficacy is expanded to a group of diverse candidates including the active camptothecin derivatives of Chemical Formula 1 or Chemical Formula 2, which are designed to bind to the DDX5 protein and an E3 ligase as molecular glues.

The camptothecin derivative of Chemical Formula 1 or 2, which is designed to bind to the DDX5 protein and an E3 ligase, according to the present invention may kill target cells that express the DDX5 protein through a molecular glue MoA.

The target cells may be cancer cells or senescent cells. Senescent cells also include cells that no longer perform an organ's specific functions.

Preferably, the camptothecin-based drug of Chemical Formula 1 or Chemical Formula 2 designed to bind to the DDX5 protein and the E3 ligase according to the present invention may have multiple mechanisms of action (MoAs) that degrade the oncoprotein DDX5 as well as the ability to inhibit topoisomerase-1.

The camptothecin derivatives represented by Chemical Formula 1 according to the present invention, for example, PBX-7011 and PBX-7012, are designed to have a pentacyclic structure with lactone in ring E essential for cytotoxicity, like camptothecin shown in FIG. 3, and maintain a lactone group and an alpha-hydroxide group located at C-20 on ring E, which are critical for the stability of a topoisomerase-1-DNA byproduct. The structural feature of the Exatecan-based drug of General Formula 1, that is, (1) the structural feature (CH₃—C═C—F) of R₁ and R₂, which has the orientation structure similar to the FL118 drug (—OCH₂O— (methylenedioxo) pentagonal ring), binding to the DDX5 protein, is maintained, and (2) various orientations of consecutive carbon-carbon single bonds of a 6- or 7-membered ring expanded from rings A and B, compared to SN-38 in which aggregation is induced by the structural feature of the exatecan drug (R₃ and R₄ of General Formula 1) is that stacking of aromatic rings formed by the π-π stacking of aromatic rings forming rings A and B are made dynamic equilibrium, thereby weakening or suppressing the stacking of aromatic rings formed by the rings A and B.

In addition, the PBX-7011 compound allows the molecular bond rotation of a carbon-carbon single bond in a hexagonal ring extending from rings A and B, which exposes —NH₂ having a large degree of freedom to water (H₂O) to either carry a (+) charge or form a hydrogen bond with water, thereby increasing water dispersibility.

In addition, the PBX-7014 compound allows the molecular bond rotation of a carbon-carbon single bond in a hexagonal ring extending from rings A and B, which exposes a lactic acid-like functional group, CH₂(OH)CONH—, of —NH₂ having a large degree of freedom to water (H₂O) to rotate like a propeller to form a hydrogen bond with water, thereby increasing water dispersibility.

Moreover, the PBX-7016 compound has a methyl group, which is a metabolically unstable functional group, introduced to the PBX-7014 compound to shorten the drug lifespan. Drugs that are highly stable in metabolism and metabolized very slowly require an appropriate retention time because they may accumulate and likely cause toxicity and serious side effects.

Meanwhile, the Dxd payload used in Enhertu was originally made from the exatecan compound, which is hardly affected by ABCG2, but was strongly affected by ABCG2 due to the presence of a glycolic acid (alpha-hydroxy acetic acid) functional group used to convert exatecan to DXd. However, the glycolic acid functional group plays a very important role in the excellent safety/efficacy profiles of Enhertu, and removing this causes difficulties in manufacturing ADCs and also deteriorates (leading to a safety problem or reduced efficacy) the performance of ADCs in animal models and clinical trials. There is a great need for a new camptothecin derivative that can be easily used in ADC preparation and is not affected by ABCG2. The PBX-7024 compound is a compound derived from the PBX-7011 compound, and a novel camptothecin compound that can be easily used in ADC preparation without being affected by ABCG2.

To confirm the anticancer efficacy of PBX-7024, efficacy was evaluated in FaDu, which is a cancer cell that does not express ABCG2, and A549, which is a cancer cell overexpressing ABCG2. It was confirmed that, in A549, which is a cancer cell line that overexpresses ABCG2, as shown in FIG. 15, PBX-7016 and PBX-7024 still maintain potent efficacy compared to camptothecin compounds including DXd, which show increased IC₅₀ values.

As shown in FIG. 28, by treating Her2-low/mid cancer cells not expressing ABCG2, FaDu, and ABCG2-overexpressing cancer cells, A549, with different concentrations of various camptothecin-based drugs, the degree of degradation of the DDX5 protein in cells and the resulting activity of inhibiting the expression of cancer-associated survival genes of survivin, Mcl-1, XIAP and cIAP2 may be confirmed, thereby indirectly comparing and confirming cell membrane permeability.

### [Anticancer Mechanism as Molecular Glue Degrader Binding to DDX5]

The ubiquitin-proteasome system (UPS) is a crucial pathway for the degradation of intracellular proteins that regulate a wide range of cellular processes. Ubiquitin is a small protein that covalently binds to a lysine residue of a substrate protein by a series of enzymatic reactions involving ubiquitin-activating enzymes (Els), ubiquitin-conjugating enzymes (E2s), and ubiquitin ligases (E3s). This process is called ubiquitination, which is a significant mechanism for regulating protein degradation, signaling, and trafficking.

Ubiquitin ligases are responsible for substrate specificity in the ubiquitination pathway.

In this regard, the camptothecin-based drug of Chemical Formula 1 or Chemical Formula 2 according to the present invention is designed to bind to the DDX5 protein and an E3 ligase.

Transformation of normal cells into cancerous cells occurs through the deregulation of different metabolic pathways involving a complex network of protein-protein interactions. Cellular enzymes and DDX5 play a critical role in maintaining a normal cellular metabolism, but when they are deregulated, tumor transformation can be accelerated. DDX5 interacts with hundreds of different cellular proteins, and depending on the specific pathway involved, both proteins can act as either tumor suppressors or oncogenes.

DDX5 (also called p68) is a multifunctional master regulator acting in the following mechanisms: (1) a biological process for co-activating the transcription of many oncogenes through direct interaction with various transcription factors (e.g., c-Myc) at oncogene promoters, and (2) a biological process for regulating miRNA and pre-RNA splicing (e.g., U1, U2, U3, . . . snRNP), and (3) ribosome biogenesis (e.g., 32S rRNA, pre-ribosome).

The camptothecin-based drug of Chemical Formula 1 or Chemical Formula 2 according to the present invention binds to the DDX5 protein without reducing DDX5 mRNA to functionally degrade the DDX5 protein through dephosphorylation and proteasomal degradation pathways, suggesting that the camptothecin-based drug of Chemical Formula 1 or Chemical Formula 2 can bind to both DDX5 and ubiquitin-involved protein stability/degradation regulators, acting as a “molecular glue degrader.”

DDX5 downstream protein targets are known to be involved in cancer initiation, development, metastasis, recurrence and treatment resistance. Accordingly, when a DDX5 downstream target is indirectly blocked through the degradation of the DDX5 protein by the camptothecin-based drug of Chemical Formula 1 or Chemical Formula 2 according to the present invention, the camptothecin-based drug of Chemical Formula 1 or Chemical Formula 2 may exhibit high antitumor efficacy.

DDX5 (p68) is a well-known multifunctional DEAD-box RNA helicase and transcription cofactor. Therefore, when cancer occurs due to the deregulation of a transcription factor by the physiological state of DDX5, cancer may be treated by selectively degrading the transcription cofactor, DDX5 (p68). Similarly, cancer may be prevented by selectively degrading the transcription cofactor, DDX5 (p68).

Since DDX5 protein is a drug target, the camptothecin-based drug of Chemical Formula 1 or Chemical Formula 2 according to the present invention may avoid drug resistance, resistance to targeted therapy, and/or resistance during treatment. In addition, the transcriptional induction of anti-apoptotic genes may be blocked by the camptothecin-based drug of Chemical Formula 1 or Chemical Formula 2. Further, the transcription cofactor DDX5 protein may be degraded by the camptothecin-based drug of Chemical Formula 1 or Chemical Formula 2, thereby maintaining or improving the sensitivity of cancer cells to chemotherapy or radiotherapy.

### [Advantages of Drug Modality Called Molecular Glue Degrader]

Proteins in cells of the body are naturally degraded within hours to days after performing their functions. All cells in the body have a purification system called the ubiquitin proteasome system (UPS) that degrades proteins, and in this process, ubiquitin acts as a marker that indicates a protein to be degraded, and the proteasome acts as a grinder that recognizes a ubiquitin marker and destroys the relevant protein. That is, several substances called ubiquitin are attached like markers to proteins that have completed their function, and the substance called proteasome selects only proteins with these markers and degrades them like a grinder. An E3 ligase is an enzyme that initiates the protein degradation system in the body, and is responsible for substrate specificity in the ubiquitination pathway.

A molecular glue degrader or molecular glue is a compound that acts as an adhesive for bonding a target protein with a certain enzyme (E3 ligase) in the body. One of the advantages of molecular glue is that it acts as a catalyst, which can degrade a target and then be dissociated to degrade another target protein.

When the E3 ligase enzyme binds to an oncoprotein through a molecular glue, the oncoprotein is degraded, and other oncoproteins are degraded sequentially until the target oncoprotein disappears, thereby preventing cancer cell proliferation. Therefore, the molecular glue for the oncoprotein overcomes drug resistance, which is a problem of targeted anticancer agents, and has a high therapeutic effect even with a low dose.

The camptothecin-based drug of Chemical Formula 1 or Chemical Formula 2 used as a payload according to the present invention is a molecular glue degrader that binds to the DDX5 protein and an E3 ligase, that is, a molecular glue that activates the degradation of the oncoprotein DDX5 or phosphorylated DDX5 protein (p-DDX5) thereof (FIGS. 3, 26 and 27).

A molecular glue may act as not only a ligand (warhead) binding to a target protein but also an E3 ligase ligand (binder).

Therefore, the camptothecin-based drug of Chemical Formula 1 or Chemical Formula 2 according to the present invention is a molecular glue activating oncoprotein DDX5 degradation, and it can be used as a ligand targeting the DDX5 protein or a ligand binding to the DDX5 protein.

A molecular glue is ‘proximity-driven’ unlike a kinase inhibitor, and its degradation-inducing ability is ‘event-driven’ depending on the formation of a temporary triple complex of ‘target protein—molecular glue-E3 ligase’. After degradation occurs, dissociated molecular glues form an additional triple complex with a target protein, allowing multiple degradations until the target protein is eliminated.

Most drug-targeted proteins acquire resistance by adapting to a drug. However, a molecular glue, which is a small molecule compound acting as an adhesive for bonding an oncoprotein with an E3 ligase, is a modality suitable for a cancer treatment because it is resistant to resistance.

Each time a genetic mutation occurs, the shape of a drug target protein slightly changes. It would be preferable to block the activity of all mutants with one drug, but this is not possible due to selectivity, so in order to block the activity of all mutants with one drug, the drug target protein is degraded and completely removed.

Therefore, the camptothecin-based drug of Chemical Formula 1 or Chemical Formula 2 according to the present invention is a drug that can precisely bind to the DDX5 oncoprotein, and can avoid the resistance of targeted therapeutics through a molecular glue approach that selectively degrades the protein.

The camptothecin-based drug of Chemical Formula 1 or Chemical Formula 2 according to the present invention is preferably an irreversible drug that binds so strongly it cannot return to its previous state, and the binding strength of the derivative to the target protein, the oncoprotein DDX5, is determined by its physicochemical properties.

Unlike the existing SN38 drug, the camptothecin-based drug of Chemical Formula 1 or Chemical Formula 2 according to the present invention may not be easily dissociated due to high affinity with DDX5, degrade DDX5 by the function of a molecular glue not only to irreversibly inhibit the signaling of cancer cells associated with DDX5 but also to regulate the cell membrane permeability of the drug according to the physicochemical properties thereof as desired, may be beneficial for treatment of heterogeneous tumors due to a high effect of killing surrounding cells by exhibiting a bystander effect or controlling the degree of its effect, may suppress the long-term progression of cancer, and may increase the treatment response rate by reducing the risk of developing resistance.

The camptothecin-based drug of Chemical Formula 1 or Chemical Formula 2 according to the present invention may bind to DDX5 acting as an oncoprotein in cells to induce cell death through DDX5 protein degradation (FIGS. 28 and 29).

In cancer treatment, intrinsic drug resistance may be caused by abnormally expressed transcription factors, which are critical regulators of cell proliferation and cell death. Therefore, the camptothecin-based drug of Chemical Formula 1 or Chemical Formula 2 according to the present invention can induce DDX5 protein degradation and resulting cell death through the MoA as a molecular glue degrader binding to DDX5, which is a transcription cofactor and an oncoprotein. Here, DDX5 protein degradation may downregulate the transcription of anti-apoptotic genes (FIGS. 26 and 27). Therefore, unlike other targeted therapeutics, drug resistance caused by abnormally expressed cell proliferation and/or cell death-related transcription factors may not develop, and/or acquired drug resistance induced through the activation of dysregulated transcription factors during chemotherapy and/or radiotherapy may be inhibited.

That is, since the anticancer mechanism of the camptothecin-based drug of Chemical Formula 1 or Chemical Formula 2 according to the present invention may bypass the molecular mechanism of cancer treatment resistance, the active camptothecin derivative can be free of the drug resistance issues. Since the therapeutic effect is lower than before resistance occurs, the camptothecin-based drug of Chemical Formula 1 or Chemical Formula 2 according to the present invention may be preferred as a standard treatment or first-line treatment after cancer diagnosis.

In short, the camptothecin-based drug of Chemical Formula 1 or Chemical Formula 2 used as a payload according to the present invention may become a targeted anticancer agent that acts on the DDX5 protein, which plays an important role in the growth, survival, proliferation, metastasis, and/or metabolism of cancer cells.

### [Use of Combination of Cytotoxic Drugs as Payload]

There are various anticancer agents to choose from to treat cancer. However, since there are not many anticancer agents that can show dramatic effects in monotherapy, combination therapy that mixes two or more agents is being widely studied.

Meanwhile, antibodies are considered an important component in determining the effects of ADCs, but a cytotoxic drug is the one that carries out tumor cell killing. Cytotoxic drugs are small molecule drugs that induce the killing of tumor cells.

In addition, when designing ADCs, it is important to adopt an effective drug that enhances specificity for target tumor cells, ensures stability in plasma to reduce toxicity to normal cells, thereby increasing the tumor cell killing effect. Even linkers that are attached to the same antibody amino acid sequence, depending on the type and length of a linker, and the location of an antibody to which the linker is conjugated, the in vivo transformation due to linker deconjugation may be affected in terms of the three-dimensional environment and electromagnetic environment. Additionally, the ADC must maintain the same affinity as the antibody before conjugation of the antibody and the drug. That is, the drug bound to the antibody must not affect antibody-antigen binding.

Taking all of the above into consideration, the ADC of the present invention is an ADC in which two types of drug-linker conjugates are linked to one antibody, and is designed such that a drug-linker conjugate (A) consisting of a combination of a camptothecin-based drug that degrades the DDX5 protein with a DAR=4 or higher, and (i) an acid-sensitive linker or (ii) an enzyme-sensitive linker; and a drug-linker conjugate (B) consisting of a combination of a non-camptothecin-based cytotoxic drug and an enzyme-sensitive linker are bound to an antibody.

Here, the drug-linker conjugate (B) consisting of a combination of a non-camptothecin-based cytotoxic drug and an enzyme-sensitive linker may be a drug-linker conjugate (B-1) consisting of a combination of a non-camptothecin-based super-toxic drug having a DAR=4 or less and an enzyme-sensitive linker or a drug-linker conjugate (B-2) consisting of a combination of an anti-apoptotic protein inhibitor drug and an enzyme-sensitive linker.

Preferably, depending on a drug-linker conjugate, it may be homogeneously and symmetrically bound to cysteine and/or lysine residues of the antibody by using different (i) amino acid residues at a site where binding to the antibody occurs, (ii) binding orders and/or (iii) binding methods.

An ADC in which two types of drug-linker conjugates are linked according to the present invention may use, as a payload, a combination of (A) a camptothecin-based drug having various pharmacological effects, particularly, a camptothecin-based drug that degrades the DDX5 protein, and preferably, an active camptothecin derivative represented by Chemical Formula 1 or Chemical Formula 2; and (B) a non-camptothecin-based cytotoxic drug, which is efficiently delivered to cancer tissue or cancer cells to maximize anticancer efficacy, thereby reducing the dose of the ADC, which can solve the problem of off-target toxicity which may occur after the non-selective uptake of a camptothecin-based drug released from apoptotic cells and/or an ADC (FIGS. 1 and 2).

After binding to a target cell, the ADC is internalized into the cell by a process called receptor-mediated endocytosis. Here, although a sufficient concentration of active drug should enter the cell, internalization by an antigen-antibody complex is generally inefficient and the number of antigens on a cell surface is generally limited to less than 1×10⁵ receptors/cell, so a very potent drug must be used to sufficiently kill tumor cells even at a low drug concentration. Accordingly, a drug that is bound to an antibody and used as an ADC is a drug with 100 to 1000 times more cytotoxicity than a commonly used anticancer agent.

As shown in FIG. 1, to this end, the present invention not only increases the killing effect of cancer cells expressing a target antigen of the ADC (IC₅₀ of Tra-25-6(DAR6)=0.5481→IC₅₀ of Tra-(MMAE)&(25-6)=0.08349 nM) even at low concentrations of drugs (payloads) through a dual payload ADC using a combination of two types of drugs (payloads), but also the dual payload ADC exhibits unexpected stability against non-target normal cells (IC₅₀ of Tra-25-6(DAR6)=97.61→IC₅₀ of Tra-(MMAE)&(25-6)=136.6 nM).

Likewise, as shown in FIG. 2, the dual payload ADC using a combination of two types of drugs (payloads) according to the present invention increases not only the killing effect of cancer cells expressing a target antigen of the ADC (IC₅₀ of Tra-25-6(DAR4)=0.4543→IC₅₀ of Tra-(Veliparib)&(25-6)=0.3626 nM) even at low concentrations of drugs (payloads), but also the dual payload ADC exhibits unexpected stability against non-target normal cells (IC₅₀ of Tra-25-6(DAR4)=240.7→IC₅₀ of Tra-(Veliparib)&(25-6)=332.9 nM).

This shows that the use of the combination of a camptothecin-based payload and a non-camptothecin-based payload (e.g., MMAE payload or veliparib payload) prevents ADC uptake by cells that do not express a target antigen. Therefore, the use of a combination with various non-camptothecin-based payloads can increase a therapeutic index in various ranges, compared to an ADC, which has the combination of the same camptothecin-based drug (the same DAR) and the same linker.

Most potent cytotoxic drugs introduced into ADCs are too toxic and also affect normal cells due to a bystander effect. In addition, the amount of payload that can be transported is limited because the drug must be conjugated while minimizing the effect on the antibody. This shows that a cytotoxic drug to be applied in ADCs must kill most tumor cells at a low concentration (nM or pM), and needs to exhibit a therapeutic effect while controlling drug release.

Since the camptothecin-based drug according to the present invention is a hydrophobic small molecule that can penetrate the cell membrane, it can be accumulated at a high concentration while penetrating deep into cancerous tissue, exhibit cytotoxicity in cells to kill the cells after passing through the cell membrane and being released, and subsequently move into surrounding cells by passing through the cell membrane to act.

Since the dual payload ADC using a combination of two types of drugs (payloads) according to the present invention has a higher target cancer cell killing effect at a lower concentration of drug (payload) than a single payload ADC (IC₅₀ of Tra-25-6(DAR6)=0.5481→IC₅₀ of Tra-(MMAE)&(25-6)=0.08349 nM) (IC₅₀ of Tra-25-6(DAR4)=0.4543→IC₅₀ of Tra-(Veliparib)&(25-6)=0.3626 nM), the concentration of the total payload is reduced, which may inhibit the bystander effect of free payload released from apoptotic cells on normal cells or overcome the off-target toxicity of the free camptothecin-based drug (payload) released from target/non-target apoptotic cells.

Therefore, when the combination of a camptothecin-based drug (A) having a DAR=4 or higher; and a non-camptothecin-based super-toxic drug (B-1) having a DAR=4 or less or an anti-apoptotic protein inhibitor drug (B-2) is used as a payload of the ADC according to the present invention, most tumor cells may be killed by the ADC at a dose of 2 to 10 mg/kg, and preferably, 4 to 10 mg/kg, and/or at a low concentration (nM or pM).

Unlike SN-38, non-limiting examples of camptothecin-based cytotoxic drugs that inhibit Bcl family members such as survivin, a resistance protein involved in a resistance mechanism, or binding to the oncoprotein DDX5 (p68) that controls c-Myc, survivin, and mutant Kras include FL118, exatecan, Dxd, and an active camptothecin derivative represented by Chemical Formula 1 or Chemical Formula 2.

In addition, the camptothecin-based cytotoxic drug degrading the DDX5 protein is designed to bind to the DDX5 protein and an E3 ligase, and may kill cells through an MoA that degrades the oncoprotein DDX5 along with the ability to inhibit topoisomerase-1.

The ADC using two types of payloads according to the present invention preferably uses the active camptothecin derivative represented by Chemical Formula 1 or Chemical Formula 2, designed to bind to the DDX5 protein and the E3 ligase as a payload for ensuring sufficient ADC efficacy at a DAR of 4.

Non-limiting examples of a non-camptothecin-based cytotoxic drug (B), which is used in combination with the camptothecin-based drug (A) degrading the DDX5 protein as an ADC payload include a tubulin disruptor, a DNA-modifying agent, and an anti-apoptotic protein inhibitor.

Microtubules play a very important role in the cell cycle. When the microtubules are disrupted, cells in the cell division phase die. DNA-modifying agents may kill cells regardless of the cell cycle. Recently, cytotoxic drugs with different concepts have been developed and used.

### [Non-Camptothecin-Based Cytotoxic Drug]

ADCs using a super toxin, such as existing MMAE, hemiasterlin, calicheamicin, and PBD, utilize a stable linker system (a characteristic of 2^{nd}-generation ADCs), which is intended to minimize the separation of the drug in blood before reaching cancer tissue.

The most commonly used tubulin disruptors (antimitotics) are the auristatin series. Monomethyl auristatin E (MMAE, also called vedotin) is a cytotoxic drug that is a derivative of dolastatin 10, which is a natural cytotoxin-like peptide isolated from Dolabella auricularia in the Indian Ocean, and a potent microtubule polymerization inhibitor. This drug is designed to be linked to a dipeptide linker in ADCs such as Adcetris or Polivy, and released by cleavage from an antibody by the cathepsin B enzyme. Another derivative, monomethyl auristatin F (MMAF), also inhibits microtubule polymerization, and was developed in a form that is linked to a non-cleavable linker and has limited cellular release. MMAF was used in Blenrep. Another microtubule inhibitor is maitansine, which binds to tubulin and inhibits the assembly of microtubules. Maitansine derivatives are called maytansinoids and include DM1, DM2, and DM4, and DM1 is used in Kadcyla. Other microtubule inhibitors being studied include tubulysins, cryptomycins, and antimitotic EG5 inhibitors.

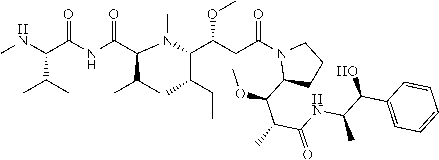 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)[C@H](C(=O)NC(=O)[C@@H](NC)C(C)C)C(C)C]

ADCs using HTI-286, a relatively safe hemiasterlin derivative, and its derivatives are being developed through microtubule inhibition, which is a representative anticancer MoA different from the topoisomerase-1 MoA.

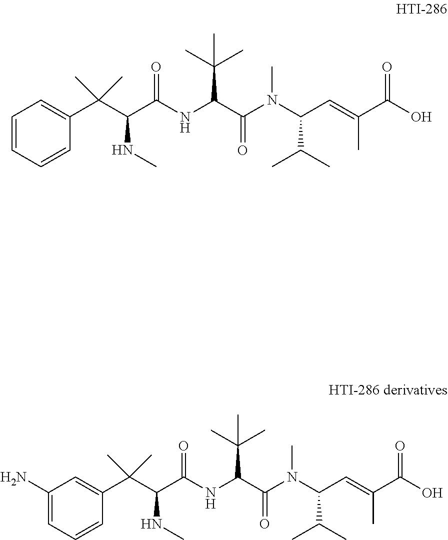

Particularly, the US company Sutro has developed an ADC using a hemiasterlin derivative, which is undergoing clinical evaluation, and this ADC shows significantly improved safety compared to MMAE-based ADCs with the same MoA (microtubule inhibition) but stronger potency.

Pyrrolobenzodiazepines (PBDs) are DNA alkylating agents and include SG3199 and SG2057. Cytotoxic drugs such as the PBD family are much more effective than existing cytotoxic drugs, but show toxicity problems when exposed to the whole body.

PARP inhibitors are a group of pharmacological inhibitors of the enzyme (poly)ADP-ribose polymerase (PARP).

PARP helps damaged cells repair themselves. As a cancer treatment, PARP inhibitors interrupt PARP-involved repair work in cancer cells to kill the cells.

PARP inhibitors were developed for several indications including hereditary cancer treatment. PARPs (PARP1 and PARP2) are attractive targets for cancer treatment because many types of cancer are rely more on PARPs than normal cells. PARP inhibitors have been shown to improve progression-free survival in women with recurrent platinum-sensitive ovarian cancer, as demonstrated primarily by the addition of olaparib to conventional treatment.

The PARP 1 and 2 inhibitor, veliparib, exhibits an antitumor action alone or in combination with a chemotherapeutic agent.

That is, veliparib is a poly(ADP-ribose) polymerase (PARP) inhibitor, which works by inhibiting the activity of the PARP enzyme serving to repair damaged DNA. Veliparib may inhibit PARP to prevent DNA repair in cancer cells, thereby killing cancer cells.

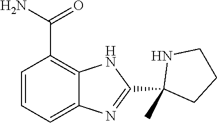 [SMILES: C[C@]1(c2nc3cccc(C(N)=O)c3[nH]2)CCCN1]

Since rapidly dividing cells, such as cancer cells, may be much more sensitive to the effects of a cytotoxic agent than slowly-dividing normal cells, the cancer cells may be removed using a DNA-modifying agent.

DNA damage is the MoA of many of the most commonly used chemotherapeutic agents, but the therapeutic window of clinically used therapeutic agents narrows as efficacy increases, making them difficult to be used as therapeutic agents due to the risk of toxicity. However, both the safety and efficacy of a drug may be increased by combining a DNA-modifying drug with strong efficacy and an antibody with high targetability.

Most DNA-modifying agents are also derived from natural substances. DNA-modifying agents include calicheamicin, pyrrolobenzodiazepine (PBD), SN-38, Dxd (exatecan derivative), camptothecin (CPT), and their derivatives.

Inducing cell death by targeting the apoptosis pathway is also a good cancer treatment. Proteins of the BCL-2 family play a central role in mitochondria-mediated apoptosis. Among them, anti-apoptotic proteins BCL-2, BCL-XL, and MCL-1 are well-known anticancer targets. Drugs targeting BCL-XL are being applied to ADCs and are undergoing clinical trials.

Anti-apoptotic proteins, which are the main cause of resistance to anticancer agents, include survivin, cIAP2, and XIAP.

Examples of anti-apoptotic protein inhibitor drugs are a Bcl-XL inhibitor, a survivin inhibitor, an MCL-1 inhibitor, and a CHK inhibitor.

Generally, the apoptosis mechanism is a series of processes in which signals are transmitted as intracellular proteins are degraded by proteases called caspases, and various types of caspases are associated with apoptosis. Caspase-8 is activated by an apoptosis-inducing substance such as a TNF-α or Fas ligand, thereby activating a series of other caspases and inducing cell death. Meanwhile, in the cell death process, cytochrome c is released through a channel present in the mitochondrial membrane and regulated by BCL-2 proteins constituting the channel. It has been reported that the released cytochrome c binds to Apaf-1, caspase-9, and dATP to activate caspase-9, and activates caspase-3 by caspase-9 to induce cell death.

There are two factors that determine tumor growth: cell proliferation and cell death. When the cell cycle of tumor cells is arrested by a cytotoxic material, the tumor cells die due to apoptosis.

### B-Cell Lymphoma (BCL)-2 is Mediated by Apoptosis.

Although cancer treatments lead to several types of cell death, the activation of the apoptosis pathway regulated by BCL-2 is most critical for the therapeutic efficacy of oncogenic kinase inhibitors and cytotoxic agents. However, the defects in the mitochondrial cell death pathway cause several types of cancer to become resistant to cytotoxic drugs.

BCL-2 overexpression has been demonstrated in chronic lymphocytic leukemia (CLL) cells to mediate tumor cell survival, and is associated with resistance to chemotherapeutic agents.

Venetoclax is a potent and selective small molecule inhibitor of the anti-apoptotic protein BCL-2. That is, Venetoclax is an apoptosis-inducing anticancer agent. Venetoclax directly binds to the BH3-binding groove of BCL-2 to replace a BH3 motif-containing pro-apoptotic protein such as BIM, and thus BIM, which is not bound to BCL-2, initiates mitochondrial membrane permeabilization (MOMP), caspase activation, and apoptosis. In non- clinical trials, this drug showed cytotoxicity in tumor cells overexpressing BCL-2.

Survivin is mainly distributed in cancer cells, so in the development of anticancer agents, a survivin inhibitor binds to survivin to inhibit its activity, thereby inducing apoptosis. Therefore, survivin can selectively act only on cancer cells, so it is highly likely to minimize side effects in the human body.

### [Linkers]

Among the components that constitute ADCs, a linker binds an antibody to a cytotoxic drug.

A linker must be stable in the bloodstream to prevent a drug from being separated from an antibody and maintain the drug in a prodrug state until it reaches a target, thereby minimizing damage to normal tissue. The most ideal linker is one that is stable when the ADC is circulated throughout the body and is cleaved in a target cell to appropriately release a cytotoxic drug, thereby safely delivering a drug to the target, ensuring that the ADC has both efficacy and safety.

When binding a drug to an antibody using a linker, the drug should not affect the structural stability, substrate binding characteristics, and pharmacokinetics of the antibody. One of the reasons for the failure of early ADC drugs is known to be the early release of a drug.

A significant number of ADCs currently in clinical trials employ chemical linkers such as hydrazone, disulfide, peptide, or thioether linkers. The process of releasing a drug from linkers utilizes a difference in specific pH or enzyme concentrations in cancer cells. Typically, hydrazone and disulfide linkers have limited stability in plasma. On the other hand, a peptide-based linker has excellent stability in plasma and facilitates the control of drug release.

Some chemical linkers regulate the balance of hydrophobicity between an antibody and a drug, preventing ADC aggregation in the bloodstream, which is a hydrophilic environment. Hydrophilic linkers and spacers include cyclodextrin, polyethylene glycol (PEG), and other polymers, which play a role in stability and pharmacodynamic properties in the bloodstream.

Linkers used in ADCs are divided into non-cleavable and cleavable types depending on their cleavage ability.

Non-cleavable linkers have relatively high plasma stability and are resistant to protein degradation. After being introduced into a cell, the antibody is degraded only to release the drug in the form of a complex with the linker, and this complex exhibits drug activity.

A representative non-cleavable linker is a thioether linker, which has higher plasma stability compared to a cleavable linker. Unlike a cleavable linker, in a non-cleavable linker, the linker itself is not degraded, so it has the characteristic that a drug can be only released when an antibody is degraded after being introduced into cells in the form of an ADC.

The drug released in the above way is charged and unlikely to diffuse to surrounding cells (bystander effect). Although there is no bystander effect that shows toxicity to surrounding cells, the drug is relatively highly safe because it affects only target cells after internalization into target cells, and it can be seen that an ADC manufactured by a non-cleavable linker is more dependent on the biological mechanism in target cells. In many studies, ADCs with non-cleavable linkers have shown high stability and efficacy, and non-cleavable linkers are being used as linkers for ADC development. Currently, the ADC to which this technology is applied is Kadcycla®.

A cleavable linker is cleaved in response to a specific environmental factor to release a drug into the cytoplasm. There are two types of cleavable linkers: enzyme-cleavable and non-enzyme-cleavable types.

An enzyme-cleavable linker is cleaved by an enzyme such as cathepsin B, β-glucuronidase, phosphatase, pyrophosphatase, or sulfatase.

A peptide linker is degraded by a protease and has high plasma stability due to the presence of a protease inhibitor in plasma. A representative protease used in ADCs is cathepsin B. Cathepsin B is present at a high level in tumor tissue, providing tumor selectivity to ADCs. A peptide linker is mainly developed as a dipeptide in which two amino acids are attached, and was applied to Adcetris®.

A peptide linker is composed of a dipeptide or tetrapeptide that is recognized and cleaved by a protease in a lysosome when an ADC is internalized. A tetrapeptide was used in the early stage of development and had limitations such as relatively slow drug release and aggregation when combined with a hydrophobic drug. These problems were solved by the development of dipeptide linkers, such as Val-Cit, Phe-Lys, Val-Lys, and Val-Ala, which were successfully applied to several ADCs such as Adcetris® and vedotin.

A β-glucuronide linker is degraded by β-glucuronidase, which is a glycolytic enzyme present in lysosomes. β-glucuronidase is overexpressed in some tumor cells to give tumor specificity.

β-glucuronidase is abundantly present in lysosomes, and is known to be overexpressed in some tumors. This enzyme is highly active at a low pH but drops to 10% at neutral pH, and thus ADCs with a β-glucuronide linker has improved stability in plasma, preventing off-target drug release. To confirm the plasma stability of the β-glucuronide linker, when an experiment was conducted in rat plasma with a Val-Cit linker, the β-glucuronide linker was much more stable, with 89% and less than 50%, respectively, after 7 days. The half-lives of the β-glucuronide linker and the Val-Cit linker were measured to be approximately 81 and 6 days, respectively. In addition, ADCs with a β-glucuronide linker exhibit high stability and efficacy even when cytotoxic drugs were combined at a high dose (up to 8).

Non-enzyme cleavable linkers include an acid-labile linker and a redox linker.

An acid-labile linker is a chemically unstable linker, which was developed in the early stage of ADC development, and is still in use today despite their low stability. A representative linker is a hydrazone linker, which is stable in the neutral environment of blood (pH 7.3 to 7.5), but has a mechanism of releasing a drug by being hydrolyzed in a weakly acidic environment such as around tumor cells (pH 6.5 to 7.2) or in endosomes (pH 5.0 to 6.5) and lysosomes (pH 4.5 to 5.0) when internalized in cells. However, since acidic conditions are not limited to the tumor microenvironment and often found outside cells, non-specific drug release may occur. Mylotarg®, which is the first ADC approved by the FDA, is a representative example that uses a hydrazone linker. However, due to its low stability in plasma, it was withdrawn from the US market in 2010 but was re-approved in 2017. Recently, a silyl ether linker has been studied, and the silyl ether linker has high stability in plasma and a half-life of more than 7 days, which is significantly improved compared to hydrazone having a half-life of 2 to 3 days.

A representative redox linker is a disulfide linker. A disulfide linker is a type of chemically unstable linker and is based on redox reactions. After internalization, a cytotoxic drug is released when the linker is degraded by disulfide exchange or a reducing agent such as glutathione.

Glutathione is a low-molecular-weight thiol, which is known as an antioxidant that regulates cell proliferation and death and protects cells from inflammation and oxidative stress. Glutathione is present at a concentration of 0.5 to 10 mM in cells, but is present in tumors under hypoxic conditions at a concentration up to 1,000 times higher. Since a disulfide linker is present at a low concentration (2 to 20 μM) in plasma and exhibits high plasma stability, non-specific drug release is reduced, making it a relatively safe and tumor-specific linker.

One of the major challenges for the development of safe and effective ADCs is developing an appropriate chemical linker between cytotoxic drugs and monoclonal antibodies. The synthesis of a linker is quite complex and the efficient release of cytotoxic drugs is affected by the type of linker used.

The development of linkers should reflect the long half-life of monoclonal antibodies (mAbs), which is an advantage, to stabilize mAbs in systemic circulation. In linker development, it is important that the combination of a linker and a cytotoxic drug does not affect the stability and pharmacokinetics of an antibody. There are many cases where several ADCs initially showing promising preclinical data failed to reach clinical development despite their potential due to the lack of an appropriate linker.

Generally, the catabolic actions that may occur during linker-cytotoxic drug-associated systemic blood circulation are as follows, and there are various other catabolic reactions that have not been identified: hydrazone cleavage, protease-mediated dipeptide cleavage, esterase-mediated carbamate cleavage, hydrolysis of acetate ester, disulfide cleavage, and succinimide ring opening.

The catabolic action that occurs at a specific site of linker-cytotoxic drugs may sometimes allow cytotoxic effects to persist and remain active at a target, and can cause toxicity during systemic blood circulation. Conversely, if the cytotoxic effect is low, it may be difficult to expect a pharmacological effect even when the drug reaches the target. For example, in the case of thailanstatin, which was developed as a payload as a spliceosome inhibitor, activity is maintained even when ester hydrolysis occurs, whereas in the case of tubulin inhibitors such as cryptophycin or tubulysin, activity is lost when ester hydrolysis occurs.

In principle, these chemical linkers induce cytotoxic drug release within cancer cells by using differences in intracellular pH, enzyme concentration, etc. Ensuring the stability of the drug-linker in the body after administration is the biggest challenge in the ADC development process, and chemically unstable hydrazone and disulfide linkers are not sufficiently stable in plasma.

Peptide-based linkers have excellent plasma stability while possessing well-controlled drug-linker stability and drug release capabilities. A peptide-based valine-citrulline linker is cleaved by the cathepsin enzyme. Cleavable dipeptide linkers such as valine-alanine (Val-Ala) and valine-citrulline (Val-Cit) are rapidly hydrolyzed in the presence of a lysosome extract or purified human cathepsin B, so the principle for linker cleavage is dependent on intracellular environments.

Meanwhile, it is difficult to deliver a sufficient amount of drug with the limited drug delivery efficiency of the existing Val-Cit or MAC-glucuronide linker system.

Brentuximab vedotin (Adcetris) is composed of a cleavable Val-Cit dipeptide linker that is selectively degraded by cathepsin B present in the lysosomes of target cancer cells to release MMAE, and is stable when circulating in the body. In ADCs using cleavable dipeptide linkers such as Val-Ala and Val-Cit, the antibody region of the ADC binds to an antigen of target cancer cells, forming an ADC-antigen complex, and then the complex is internalized into the cancer cell through the endosomal-lysosomal pathway. In this case, the intracellular release of a cytotoxic drug is regulated by the internal environment of the endosome/lysosome. That is, a hydrazone linker is unstable in acidic conditions and releases a drug upon degradation, and the Val-Cit dipeptide linker releases MMAE by cathepsin B, which is a protease in lysosomes.

On the other hand, ado-trastuzumab emtansine (Kadcyla) has a non-cleavable SMCC linker. A non-cleavable linker can avoid unnecessary drug release in the body by releasing a cytotoxic drug when the ADC introduced into a target cell is degraded by lysosomes as well as modify the chemical properties of the bound drug to adjust its affinity for a carrier or improve the efficacy of the drug.

ADC stability during delivery to a target is very important to achieving the desired therapeutic index, regardless of the type of linker used, that is, whether a cleavable or non-cleavable linker is used.

In the present invention, the linkage of [linker]-[antibody] may be a linkage in which a thiol group contained in the antibody is bonded to a maleimide group or maleic hydrazide group of the linker through a “click” reaction of Reaction Scheme 1.

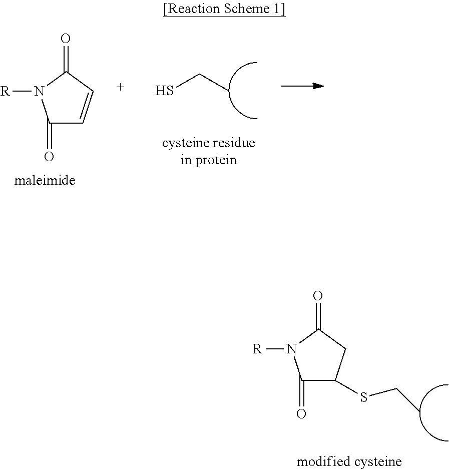

Linkers having a sulfonate- or PEG-containing hydrophilic spacer, which exhibits high solubility in both organic and aqueous solutions, solve several problems observed in a hydrophobic linker. PEG linkers have the advantages of water solubility, low toxicity, low immunogenicity, and a controlled linker chain length. In this regard, studies have reported that the use of PEG linkers significantly improves an in vivo pharmacokinetic profile, increases the half-life and plasma concentration, and increases the plasma concentration-time curve (AUC).

### [Enzyme-Sensitive Linker]

An enzyme-sensitive linker according to one embodiment of the present invention may be —S-maleimide-spacer-enzymatic cleavable site-self-immolative spacer-(payload) or —S-dibromaleimide-spacer-enzymatic cleavable site-self-immolative spacer-(payload). In Examples 2 to 5, ADCs with DAR 8 or DAR 4 were prepared using the enzyme-sensitive linker.

Here, non-limiting examples of the self-immolative spacer are shown in FIG. 19.

Biotransformation caused by linker deconjugation may occur by chemical dissociation or enzymatic cleavage.

In the case of ADCs in which a linker is connected to a lysine residue by an amide bond the linker-cytotoxic drug may be detached due to enzymatic amide hydrolysis. In the case of ADCs in which a linker containing a maleimide group or a linker containing a disulfide group is connected to a cysteine residue, the S of the cysteine residue may be reduced, and a linker-cytotoxic drug conjugated in the exchange manner may be detached. After being detached, the cysteine residue of the monoclonal antibody may be present in the state of being disulfide-bonded with another cysteine amino acid or an endogenous or exogenous substance containing S such as glutathione (GSH). Therefore, ADCs may lose the MoA on a target due to the cytotoxic drug, and the detached linker-cytotoxic drug may form an adduct on another protein or enzyme or be metabolized to exhibit activity and cause toxicity.

### [Acid-Sensitive Linker]

In the present invention, an acid-sensitive linker refers to a linker that is stable in the neutral environment of blood (pH 7.3 to 7.5), but is hydrolyzed in a weakly acidic environment such as around tumor cells (pH 6.5 to 7.2) or in endosomes (pH 5.0 to 6.5) and lysosomes (pH 4.5 to 5.0) when internalized in cells, thereby releasing a drug. Accordingly, in the present invention, the acid-sensitive linker has a hydrophilic molecular structure to create a hydrolysis environment. To this end, the acid-sensitive linker may include, for example, a polyethylene glycol (PEG) spacer.

It is preferable that a camptothecin-based drug and an acid-sensitive linker are connected by a carbonate or ester bond so that the free camptothecin-based drug is released by degradation of the acid-sensitive linker in an acidic environment (pH≤7).

A tetrapeptide linker showed a limitation in which ADC aggregation can occur when combined with a hydrophobic drug.

CL2A, which is the linker used in the existing FDA-approved ADC, Trodelvy, is a linker that satisfies all of the following characteristics: (i) storage stability after manufacturing, (ii) stability in blood upon administration (almost no exposure of free payload in plasma), and (iii) the rapid release of payload in cancer tissue.

The Phe-Lys peptide inserted into the initial CL2 derivative enables cleavage by cathepsin B. As part of an effort to simplify the synthesis process, phenylalanine was removed from CL2A, thereby removing the cathepsin B cleavage site. This change did not affect conjugate binding, stability, or efficacy. This suggests that release from the conjugate is primarily due to cleavage of the pH-sensitive benzyl carbonate bond to the lactone ring of SN-38, rather than at the cathepsin B cleavage site in CL2.

The acid-sensitive linker used in the present invention may utilize a CL2A linker, and may be designed as in Chemical Formula 3 to efficiently deliver the camptothecin-based drug to cancer tissue. That is, in the present invention, the acid-sensitive linker may be derived from a compound of Chemical Formula 3 below:

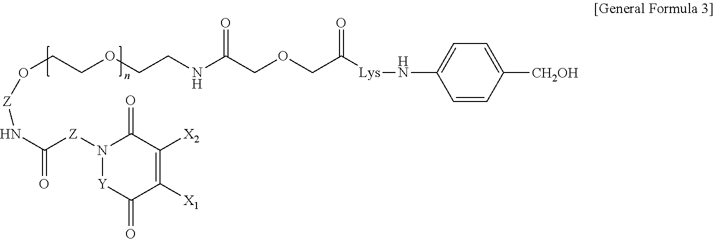

Here, X₁ and X₂ are each independently —H or -halogen;
Y is —NH—, —NR^{A}—, or nothing (null);
Z is —C₁-C₄ alkyl-, —C₃-C₆ cycloalkyl-, —(C₁-C₂ alkyl)-(C₃-C₆ cycloalkyl)-, —(C₃-C₆ cycloalkyl)-(C₁-C₂ alkyl)-, or —(C₁-C₂ alkyl)-(C₃-C₆ cycloalkyl)-(C₁-C₂ alkyl)-;
W is —R^{B}—, -M- —R^{B}-M-, -M-R^{B}—, or —R^{B}-M-R^{C};
R^{A} to R^{C} are each independently a C₁-C₄ alkyl;
M is

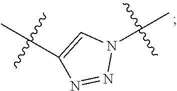 [SMILES: *c1cn(*)nn1]

and
n is an integer from 5 to 9.

Preferably, in Chemical Formula 3,
X₁ and X₂ are each independently —H or -halogen;
Y is —NR^{A}—, or null;
Z is —C₁-C₄ alkyl-, —(C₁-C₂ alkyl)-(C₃-C₆ cycloalkyl)-, or —(C₃-C₆ cycloalkyl)-(C₁-C₂ alkyl)-;
W is —R^{B}— or —R^{B}-M-R^{C}—;
R^{A} to R^{C} are each independently a C₁-C₄ alkyl;
M is

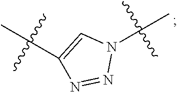 [SMILES: *c1cn(*)nn1]

and
n is an integer from 5 to 9.

In the present invention, the length of a linker, that is, n in Chemical Formula 3, may be an integer from 5 to 9, specifically, n is an integer from 6 to 8, and more specifically, n is 7, but the present invention is not limited thereto. Even when the linker length is outside the above range, when there is no significant difference in effect according to the change in linker length, it is naturally included within the equivalent scope of the present invention.

Since the water dispersibility of the drug may be improved by placing a polyethylene glycol (PEG) spacer between the drug and the antibody, the linker of Chemical Formula 3 includes a low-molecular-weight PEG moiety including a limited number (n=5 to 9) of PEG monomers.

The acid-sensitive linker of Chemical Formula 3 is a customized linker that can be optimized based on the characteristics of a target, a payload, and a carrier.

The camptothecin-based drug has sites that are easy to attach to various linkers for ADC production. For example, the alcohol group region of the camptothecin-based drug may be used as an attachment site to a linker.

Therefore, in the present invention, the [camptothecin-based drug]-[acid-sensitive linker] may include an alcohol group region of the camptothecin-based drug, which is linked with an alcohol group region of the acid-sensitive linker of Chemical Formula 3.

### [MoA of ADC Including Drug-Linker Conjugate (A) of Combination of Camptothecin-Based Drug and Acid-Sensitive Linker]

In the specification, the term “immunoconjugate” refers to a complex in which a cytotoxic drug-linker conjugate is connected to an antibody or an antigen-binding fragment thereof, and is included in the category of ADCs of the present invention.

Since an ADC is an example of immunoconjugate, in the present invention, the description of the ADC and the description of the immunoconjugate may be used interchangeably.

When administered in vivo, the immunoconjugate may release after the drug after one component of the immunoconjugate, such as the antibody or its antigen-binding site-containing fragment, binds to a targeted antigen, thereby allowing the drug to act on target cells and/or surrounding cells, and thus excellent efficacy and reduced side effects as a target drug can be expected.

Factors that have a significant effect on the effects of ADCs include, particularly, (1) drug potency, (2) drug linker stability, and (3) efficient on-target drug release. Since multiple factors affect the ADC effects in a complex manner, it is extremely difficult to predict the effects of ADCs, which is the combination of these factors, based only on known facts about each factor.

In an immunoconjugate including [camptothecin-based drug degrading the DDX5 protein]-[acid-sensitive linker]-[antibody or antigen-binding site-containing fragment]; or a carrier-drug conjugate including [camptothecin-based drug degrading the DDX5 protein]-[acid-sensitive linker] according to the present invention, since at least a part of the camptothecin-based drug degrading the DDX5 protein is released by degrading the acid-sensitive linker in the acidic environment around a tumor (pH≤7), to exert an appropriate bystander effect or control the degree of its effect while penetrating deep into the tumor tissue, the relative hydrophilic/hydrophobic properties of the camptothecin-based drug have a significant impact on drug solubility, absorption, distribution, metabolism, and excretion (ADME). Particularly, it is important for how easily the drug passes through the cell membrane, and for the interaction with the drug targets, such as the DDX5 protein and/or the E3 ligase.

ADCs designed to release the drug after internalization have the problem that a sufficient concentration of the active drug cannot be delivered into cells when the internalization process is insufficient, and have the disadvantage that it is difficult to expect the bystander effect on surrounding cells even when a hydrophobic drug is adopted as a cytotoxic drug.

To solve these problems, the ADC including the drug-linker conjugate (A) of the combination of a camptothecin-based drug degrading the DDX5 protein and an acid-sensitive linker according to the present invention uses
(i) an acid-sensitive linker to introduce a large amount of free hydrophobic drug into cells after releasing a hydrophobic drug that can penetrate the cell membrane and play an appropriate role within the cells in a tumor microenvironment (pH≤7) around a tumor; and
(ii) a camptothecin-based drug, as a hydrophobic drug, which can penetrate the cell membrane to release the drug by degrading an acid-sensitive linker in the acidic environment (pH≤7) around cancer cells and allow a large amount of the free drug to penetrate the cell membrane and be concentrated within the cells (FIG. 6).

The present inventors synthesized a new ADC (ADC (DAR=8) synthesized in Preparation Example 8, named PBX-001) with a novel camptothecin-based payload, FL118, having excellent in vitro/in vivo antitumor effects and an excellent safety profile (FIGS. 6 to 16). The use of a hydrophilic CL2A linker system ensures minimal aggregation of the ADC despite the fact that FL118 itself is very hydrophobic. The camptothecin-based FL118 drug, a TOP1 inhibitor, may be conjugated to a cysteine residue of an antibody with reduced disulfide (—S—S—) without aggregation up to a DAR of 8 using a hydrophilic linker.

Sacituzumab FL118 (PBX-001) is an ADC consisting of the humanized anti-Trop2 monoclonal antibody hRS7, the novel topoisomerase-1 inhibitor FL118, and a CL2A linker system. PBX-001 may have a high DAR of approximately 7 to 8 and very efficiently release the FL118 payload in a tumor microenvironment with a low pH value. Surprisingly, as shown in FIG. 10, even when DAR=8, PBX-001 exhibited excellent serum stability compared to Trodelvy when comparing payload release amounts in human serum despite using the same CL2A linker system and hRS7 antibody. The in vitro and in vivo assessments of PBX-001 showed better efficacy than Trodelvy. In addition, non-human primate toxicity studies demonstrated the excellent safety of PBX-001.

In addition, since the FL118 payload exhibited a safety profile similar to that of the Trodelvy payload SN-38, it was confirmed in mouse and monkey models that the CL2A linker system with a high-efficiency release profile in a tumor microenvironment may be utilized without causing serious toxicity.

Since the camptothecin-based drug degrading the DDX5 protein is a hydrophobic small molecule that can penetrate the cell membrane, it may rapidly accumulate in cancer tissue and maintain a high concentration for a long time, diffuse into cells and exert cytotoxicity to kill the cells, and then be released to continuously move into surrounding cells by penetrating the cell membrane (FIGS. 30, 31, 32, 33, 34, and 35).

According to the present invention, the ADC including a drug-linker conjugate (A) consisting of the combination of a camptothecin-based drug degrading the DDX protein and an acid-sensitive linker has the technical feature of combining a camptothecin-based drug and an acid-sensitive linker as described above, so an immunoconjugate may be designed by combining any antibody or its antigen-binding site-containing fragment according to a desired purpose, all of which are incorporated within the scope of the present invention. For example, for excellent anticancer effects, an ADC may be prepared by bonding trastuzumab, cetuximab, and sacituzumab to the camptothecin-based drug-acid-sensitive linker conjugate of the present invention.

Antibodies linked to the camptothecin-based drug degrading the DDX5 protein by an acid-sensitive linker (Preparation Example 6 to 8) according to the present invention may bind to antigens overexpressed on the surface of cancer cells in the same manner as the first step in which second-generation ADCs act on cancer cells, but some of them undergo an intracellular processing step like the second-generation ADCs and a considerable portion of them may release the drug due to low pH around the cancer cells (FIG. 6). Afterward, the drug released from the cancer tissue move into cancer cells by diffusion, bypasses endosomes and lysosomes and acts directly on cancer cells without an enzymatic (cathepsin B) reaction, thereby inducing cell death. Therefore, the main feature of the immunoconjugate of the present invention is that, compared to second-generation ADCs that enable drug release only by an enzymatic reaction, although the antigen selectivity of cancer cells is the same, the drug release and delivery efficiency into the cancer cells may be maximized using a pH-sensitive linker.

In addition, the linker should be stable in the bloodstream to prevent the drug from being separated from the antibody and maintained in a prodrug state until it reaches the target, thereby minimizing damage to normal tissue. However, in the present invention, by using an acid-sensitive linker having a hydrophilic molecular structure to create a hydrolytic environment, the problem of ADC aggregation occurring when combined with a hydrophobic drug can be alleviated.

Different catabolites are formed depending on the type of linker. In this regard, in the [camptothecin-based drug degrading the DDX5 protein]-[acid-sensitive linker] of the present invention, the camptothecin-based drug is preferably connected to the acid-sensitive linker by carbonate or ester bonds, allowing the release of the free camptothecin-based drug upon the degradation of the acid-sensitive linker.

Generally, in terms of hydrolysis, carbamate bonds provide excellent drug linker stability compared to ester and carbonate bonds. However, the present invention uses unstable ester or carbonate bonds, instead of carbonate bonds, to design the camptothecin-based drug to be separated from the drug linker both around and in cancer cells in the acidic environment (pH) surrounding the cancer cells.

Blood pH is maintained at a constant level of 7.3 to 7.4. Accordingly, the camptothecin-based drug is not cleaved from the acid-sensitive linker in blood, and even if cleaved, the release rate of the camptothecin-based drug at the neutral pH of serum is significantly reduced compared to that in tumor tissue in an acidic environment.

In addition, to reduce ADC aggregation in plasma due to the hydrophobic drug, the present invention may provide a camptothecin-based drug in which the acid-sensitive linker is connected to an alpha-hydroxide group located at C-20 on ring E.

[MoA of ADC Including Drug-Linker Conjugate (A) Composed of Combination of Camptothecin-Based Drug and Acid-Sensitive Linker; and Drug-Linker Conjugate (B) Composed of Combination of Non-Camptothecin-Based Super Toxic Drug and Enzyme-Sensitive Linker]

Drug release from Group A of FIG. 20 proceeds rapidly to attack cancer cells first, and by releasing a super toxin with slow and strong anticancer efficacy from Group B, the cancer cells are attacked secondarily to cause cancer cell death. For example, while a super toxin, such as MMAE and hemiasterlin, with a DAR of 4 or more may not be used in ADC preparation due to toxicity, MMAE with a DAR of 4 or less may be used, and the insufficient toxicity may be resolved with a, camptothecin-based drug, which is a TOP1 inhibitor.

Drug-linkers of Group A and Group B may be conjugated to all disulfide bonds exposed on the outside of a single antibody, and here, the DAR may be 8 (Group A: 4 to 7, Group B: 1 to 4). A non-limiting example of the drug-linker is (MMAE-Cit-Val)₂-trastuzumab-(CL2A-FL118)₆.

[MoA of ADC Including Drug-Linker Conjugate (A) Composed of Combination of Camptothecin-Based Drug and Enzyme-Sensitive Linker; and Drug-Linker Conjugate (B) Composed of Combination of Non-Camptothecin-Based Super Toxic Drug and Enzyme-Sensitive Linker]

As both Group A and Group B of FIG. 20 can be introduced into cancer cells at relatively low rates (continuously), a camptothecin-based drug degrading the DDX5 protein may compensate for the disadvantage of a super toxin, which cannot be used at a DAR of 4 or higher, due to its toxicity. Group A and Group B may be competitively delivered into the cancer cells to cause cancer cell death.

Drug-linkers of Group B and Group C may be conjugated to all disulfide bonds exposed on the outside of a monoclonal antibody, and here, the DAR may be 8 (Group A: 4 to 7, Group B: 1 to 4), and a non-limiting example is (MMAE-Cit-Val)₂-trastuzumab-(GGFG-Dxd)₆.

### [Target Antigen]

The interaction between an antibody in the ADC and a target antigen is important to ensure safety and obtain a therapeutic effect. Two variables in antigen selection are tumor specificity and expression level. Ideally, the antigen is specifically expressed only in tumors, with no or minimal expression in normal cells. Specificity is crucial to reducing toxicity and determines the success of ADCs. A cancer-specific antigen is expressed as a surface receptor on the surface of a tumor cells or within tumor vasculature and the tumor microenvironment.

As in homogeneous tumors, a cancer-specific antigen is homogeneously expressed in tumor tissue, so that all cancer cells respond to drugs, making cancer treatment easier. In the case of heterogeneous tumors, non-responsive cancer cells may be mixed in, so there may be cancer cells that survive after ADC treatment. When the ADC has a bystander effect, the problem of heterogeneous tumors may be overcome.

Monoclonal antibodies used in ADCs include IgG1, IgG2, and IgG4, wherein IgG1 is most commonly used. In traditional ADCs, full-length antigens are used. As a strategy to enhance absorption and penetration, attempts are being made to use smaller Fabs, scFvs, and diabodies instead of monoclonal antibodies.

Among the ADCs currently undergoing clinical trials, a target antigen that is attractive due to excellent marketability is known to be HER2, and three HER2-targeting ADCs are in phase 3 clinical trials (Dean et al., 2021).

Targeting antigens internalized in tumor cells is challenging in solid tumors abundant in intracellular matrix. In this case, the approach targets the tumor microenvironment rather than cancer cells. A cytotoxic drug is released from the outside of the cell using an extracellular protein or an acidic substance of the extracellular matrix, or a component such as glutathione.

In order to deliver a strong cytotoxic drug only to specific cancer cells, determining an antigen to be targeted is the first major step in ADC development. By using an antibody that has high specificity to a target and a long half-life, long-term systemic circulation is possible, which allows the cytotoxic drug to accumulate selectively only in tumor cells, minimizes exposure to normal tissue to reduce damage and adverse effects, and increases therapeutic effects. To this end, a target antigen that can identify tumor cells should be found, and the following conditions are required. First, the target antigen must be uniformly overexpressed on the tumor cell surface and exhibit relatively low or no expression in normal cells. As a representative example, there is a human epidermal growth factor receptor 2 (HER2) receptor, which is known to be expressed more than 100 times more in HER2-positive breast cancer, compared to normal cells. Therefore, before producing an antibody, the overexpression of a specific antigen is confirmed by analyzing the tumor expression of a target antigen through various types of profiling, and then monoclonal antibodies that recognize the antigen are produced. Second, in terms of the binding force to the antigen, due to the characteristic of the antibody where internalization mediated by a receptor occurs, the stronger the binding force to an epitope of the antigen, the more internalization occurs, which increases the therapeutic effect. An additional condition is low immunogenicity. Initially, first-generation ADCs were produced using antibodies produced from mice. The first-generation ADCs had difficulty in exhibiting an anticancer effect due to adverse effects and antibody neutralization occurring due to the immune response in the human body to the administered mouse antibody after injecting the mouse antibody into a human. Problems of immune responses have been greatly improved with the development of genetic engineering technology, which has led to the production of a chimeric antibody, a humanized antibody, and a fully human antibody.

Antibodies recognizing antigens on cancer cells must be internalized into the cells along with a drug. To increase cell internalization in the cancer cells, bispecific antibodies are being developed.

MEDI4267 (trastuzumab-META) in development by Medimmune and Astrazeneca is a biparatopic antibody targeting two non-overlapping epitopes on HER2 has been shown to induce HER2 receptor clustering, thereby promoting cellular internalization, lysosomal trafficking, and degradation.

In addition, bispecific antibodies in which a lysosomal marker CD63 or APLP2, and a prolactin receptor are introduced together with a tumor target antibody showed improved cellular internalization compared to single antibodies, as well as tumor antigen recognition. There are HER2-targeting trastuzumab and a bispecific ADC of CD63 (HER2×CD63-duostatin-3, Creative Biolabs), which lower delivery to normal tissue and specifically deliver to cancer cells, thereby exhibiting a strong anticancer effect.

A targeting subject in the design of the ADC or immunoconjugate of the present invention may be expanded to not only cancer cells but also infectious disease organisms and/or autoimmune disease-associated cells.

Therefore, cells targeted by an antibody or its antigen-binding site-containing fragment may be cancer cells, infectious disease organisms, and/or autoimmune disease-associated cells.

Non-limiting examples of target antigens include antigens selectively distributed on the surface of cancer, such as Her2, FolR, and PSMA, and cancer cell-overexpressing antigens that are distributed in small numbers even in normal tissue, such as Trop 2.

A cancer cell-targeting antigen may be, for example, 5T4, ABL, ABCF1, ACVR1, ACVR1B, ACVR2, ACVR2B, ACVRL1, ADORA2A, AFP, aggrecan, AGR2, AICDA, AIF1, AIGI, AKAP1, AKAP2, ALCAM, ALK, AMH, AMHR2, ANGPT1, ANGPT2, ANGPTL3, ANGPTL4, ANPEP, APC, APOCl, AR, aromatase, ASPH, ATX, AX1, AXL, AZGP1 (zinc-a-glycoprotein), B4GALNT1, B7, B7.1, B7.2, B7-H1, B7-H3, B7-H4, B7-H6, BAD, BAFF, BAG1, BAI1, BCR, BCL2, BCL6, BCMA, BDNF, BLNK, BLR1 (MDR15), BIyS, BMP1, BMP2, BMP3B (GDFIO), BMP4, BMP6, BMP8, BMP10, BMPR1A, BMPR1B, BMPR2, BPAG1 (plectin), BRCA1, C19orflO (IL27w), C3, C4A, C5, C5R1, CA6, CA9, CANT1, CAPRIN-1, CASP1, CASP4, CAVI, CCBP2 (D6/JAB61), CCL1 (1-309), CCLI1 (eotaxin), CCL13 (MCP-4), CCL15 (MIP-Id), CCL16 (HCC-4), CCL17 (TARC), CCL18 (PARC), CCL19 (MIP-3b), CCL2 (MCP-1), MCAF, CCL20 (MIP-3a), CCL21 (MEP-2), SLC, exodus-2, CCL22(MDC/STC-I), CCL23 (MPIF-I), CCL24 (MPIF-2/eotaxin-2), CCL25 (TECK), CCL26 (eotaxin-3), CCL27 (CTACK/ILC), CCL28, CCL3 (MIP-Ia), CCL4 (MIPIb), CCL5 (RANTES), CCL7 (MCP-3), CCL8 (mcp-2), CCNA1, CCNA2, CCND1, CCNE1, CCNE2, CCR1 (CKR1/HM145), CCR2 (mcp-IRB/RA), CCR3 (CKR3/CMKBR3), CCR4, CCR5 (CMKBR5/ChemR13), CCR6 (CMKBR6/CKR-L3/STRL22/DRY6), CCR7 (CKR7/EBI1), CCR8 or CDw198 (CMKBR8/TERI/CKR-L1), CCR9 (GPR-9-6), CCRL1 (VSHK1), CCRL2 (L-CCR), CD13, CD164, CD19, CDH6, CDIC, CD2, CD20, CD21, CD200, CD22, CD23, CD24, CD27, CD28, CD29, CD3, CD33, CD35, CD37, CD38, CD3E, CD3G, CD3Z, CD4, CD40, CD40L, CD44, CD45RB, CD47, CD52, CD56, CD69, CD70, CD72, CD74, CD79A, CD79B, CD8, CD80, CD81, CD83, CD86, CD97, CD99, CD117, CD125, CD137, CD147, CD179b, CD223, CD279, CD152, CD274, CDH1 (E-cadherin), CDH1O, CDH12, CDH13, CDH18, CDH19, CDH2O, CDH3, CDH5, CDH7, CDH8, CDH9, CDH17, CDK2, CDK3, CDK4, CDK5, CDK6, CDK7, CDK9, CDKN1A (p21Wap1/Cip1), CDKN1B (p27Kip1), CDKN1C, CDKN2A (p16INK4a), CDKN2B, CDKN2C, CDKN3, CEA, CEACAM5, CEACAM6, CEBPB, CERI, CFC1B, CHGA, CHGB, chitinase, CHST1O, CIK, CKLFSF2, CKLFSF3, CKLFSF4, CKLFSF5, CKLFSF6, CKLFSF7, CKLFSF8, CLDN3, CLDN6, CLDN7 (claudin-7), CLDN18, CLEC5A, CLEC6A, CLEC11A, CLEC14A, CLN3, CLU (clusterin), CMKLR1, CMKOR1 (RDC1), CNR1, C-MET, COL18A1, COLIA1, COL4A3, COL6A1, CR2, Cripto, CRP, CSF1 (M-CSF), CSF2 (GM-CSF), CSF3 (GCSF), CTAG1B (NY-ESO-1), CTLA4, CTL8, CTNNB1 (b-catenin), CTSB (cathepsin B), CX3CL1 (SCYD1), CX3CR1 (V28), CXCL1 (GRO1), CXCL1O (IP-IO), CXCLI1 (1-TAC/IP-9), CXCL12 (SDF1), CXCL13, CXCL14, CXCL16, CXCL2 (GRO2), CXCL3 (GRO3), CXCL5 (ENA-78/LIX), CXCL6 (GCP-2), CXCL9 (MIG), CXCR3 (GPR9/CKR-L2), CXCR4, CXCR6 (TYMSTR/STRL33/Bonzo), CYB5, CYC1, CYSLTR1, DAB2IP, DES, DKFZp451J0118, DLK1, DNCL1, DPP4, E2F1, Engel, Edge, Fennel, EFNA3, EFNB2, EGF, EGFR, ELAC2, ENG, Enola, ENO2, ENO3, EpCAM, EPHA1, EPHA2, EPHA3, EPHA4, EPHA5, EPHA6, EPHA7, EPHA8, EPHA9, EPHA10, EPHB1, EPHB2, EPHB3, EPHB4, EPHB5, EPHB6, EPHRIN-A1, EPHRIN-A2, EPHRINA3, EPHRIN-A4, EPHRIN-A5, EPHRIN-A6, EPHRIN-B1, EPHRIN-B2, EPHRIN-B3, EPHB4, EPG, ERBB2 (HER-2), ERBB3, ERBB4, EREG, ERK8, estrogen receptor, Earl, ESR2, F3 (TF), FADD, FAP, farnesyl transferase, FasL, FASNf, FCER1A, FCER2, FCGR3A, FGF, FGF1 (aFGF), FGF10, FGF1 1, FGF12, FGF12B, FGF13, FGF14, FGF16, FGF17, FGF18, FGF19, FGF2 (bFGF), FGF20, FGF21, FGF22, FGF23, FGF3 (int-2), FGF4 (HST), FGF5, FGF6 (HST-2), FGF7 (KGF), FGF8, FGF9, FGFR1, FGFR2, FGFR3, FGFR4, FIGF (VEGFD), FIL1 (EPSILON), FBL1 (ZETA), FLJ12584, FLJ25530, FLRT1 (fibronectin), FLT1, FLT-3, FOLR1, FOS, FOSL1(FRA-1), FR-alpha, FY (DARC), GABRP (GABAa), GAGEB1, GAGEC1, GALNAC4S-6ST, GATA3, GD2, GD3, GDF5, GFI1, GFRA1, GGT1, GM-CSF, GNAS1, GNRH1, GPC1, GPC3, GPNB, GPR2 (CCR10), GPR31, GPR44, GPR81 (FKSG80), GRCC1O (C1O), GRP, GSN (Gelsolin), GSTP1, GUCY2C, HAVCR1, HAVCR2, HDAC, HDAC4, HDAC5, HDAC7A, HDAC9, hedgehog, HER3, HGF, HIF1A, HIP1, histamine and a histamine receptor, HLA-A, HLA-DR, HLA-DRA, HLA-E, HM74, HMOXI, HSP90, HUMCYT2A, ICEBERG, ICOSL, ID2, IFN-a, IFNA1, IFNA2, IFNA4, IFNA5, EFNA6, BFNA7, IFNB1, IFN-gamma, IFNW1, IGBP1, IGF1, IGFIR, IGF2, IGFBP2, IGFBP3, IGFBP6, DL-1, ILIO, ILIORA, ILIORB, IL-1, IL1R1 (CD121a), IL1R2(CD121b), IL-IRA, IL-2, IL2RA (CD25), IL2RB (CD122), IL2RG (CD132), IL-4, IL-4R (CD123), IL-5, IL5RA (CD125), IL3RB (CD131), IL-6, IL6RA, (CD126), IR6RB (CD130), IL-7, IL7RA(CD127), IL-8, CXCR1 (IL8RA), CXCR2, (IL8RB/CD128), IL-9, IL9R(CD129), IL-10, IL10RA(CD210), IL10RB (CDW210B), IL-11, IL11RA, IL-12, IL-12A, IL-12B, IL-12RB1, IL-12RB2, IL-13, IL13RA1, IL13RA2, IL14, IL15, IL15RA, IL16, IL17, IL17A, IL17B, IL17C, IL17R, IL18, IL18BP, IL18R1, IL18RAP, IL19, ILIA, ILIB, ILIF10, ILIF5, IL1F6, ILIF7, IL1F8, DL1F9, ILIHYI, ILIR1, IL1R2, ILIRAP, ILIRAPLI, ILIRAPL2, ILIRLI, IL1RL2, ILIRN, IL2, IL20, IL20RA, IL21R, IL22, IL22R, IL22RA2, IL23, DL24, IL25, IL26, IL27, IL28A, IL28B, IL29, IL2RA, IL2RB, IL2RG, IL3, IL30, IL3RA, IL4, IL4, IL6ST (glycoprotein 130), ILK, INHA, INHBA, INSL3, INSL4, IRAK1, IRAK2, ITGA1, ITGA2, ITGA3, ITGA6 (α6 integrin), ITGAV, ITGB3, ITGB4 (β4 integrin), JAG1, JAK1, JAK3, JTB, JUN, K6HF, KAI1, KDR, KIT, KITLG, KLF5 (GC Box BP), KLF6, KLK10, KLK12, KLK13, KLK14, KLK15, KLK3, KLK4, KLK5, KLK6, KLK9, KRT1, KRT19 (keratin 19), KRT2A, KRTHB6 (hair-specific type II keratin), LiCAM, LAG3, LAMA5, LAMP1, LEP (leptin), Lewis Y antigen (“LeY”), LILRB1, Lingo-p75, Lingo-Troy, LGALS3BP, LRRC15, LPS, LTA (TNF-b), LTB, LTB4R (GPR16), LTB4R2, LTBR, LY75, LYPD3, MACMARCKS, MAG or OMgp, MAGEA3, MAGEA6, MAP2K7 (c-Jun), MCP-1, MDK, MIB1, midkine, MIF, MISRII, MJP-2, MLSN, MK, MKI67 (Ki-67), MMP2, MMP9, MS4A1, MSMB, MT3 (metallothionein-UI), mTOR, MTSS1, MUC1 (mucin), MUC16, MYC, MYD88, NCK2, NCR3LG1, neurocan, NFKBI, NFKB2, NGFB (NGF), NGFR, NgR-Lingo, NgRNogo66, (Nogo), NgR-p75, NgR-Troy, NMEI (NM23A), NOTCH, NOTCH1, NOTCH3, NOX5, NPPB, NROB1, NROB2, NRID1, NR1D2, NR1H2, NR1H3, NR1H4, NR112, NR113, NR2C1, NR2C2, NR2E1, NR2E3, NR2F1, NR2F2, NR2F6, NR3C1, NR3C2, NR4A1, NR4A2, NR4A3, NR5A1, NR5A2, NR6A1, NRP1, NRP2, NT5E, NTN4, NY-ESO1, ODZI, OPRDI, P2RX7, PAP, PART1, PATE, PAWR, P-cadherin, PCA3, PCD1, PD-L1, PCDGF, PCNA, PDGFA, PDGFB, PDGFRA, PDGFRB, PECAMI, L1-CAM, peg-asparaginase, PF4 (CXCL4), PGF, PGR, phosphacan, PIAS2, PI3 kinase, PIK3CG, PLAU (uPA), PLG, PLXDCI, PKC, PKC-beta, PPBP (CXCL7), PPID, PR1, PRAME, PRKCQ, PRKD1, PRL, PROC, PROK2, PSAP, PSCA, PSMA, PTAFR, PTEN, PTHR2, PTGS2 (COX-2), PTN, PVRIG, RAC2 (P21Rac2), RANK, RANK ligand, RARB, RGS1, RGS13, RGS3, RNFI1O (ZNF144), Ron, ROBO2, ROR1, RXR, S100A2, SCGB 1D2 (ripopilrin B), SCGB2A1 (mammaglobin 2), SCGB2A2 (mammaglobin 1), SCYE1 (endothelial monocyte-activating cytokine), SDF2, SERPENA1, SERPINA3, SERPINB5 (maspin), SERPINEI (PAI-I), SERPINFI, SHIP-1, SHIP-2, SHB1, SHB2, SHBG, SfcAZ, SLAMF7, SLC2A2, SLC33A1, SLC43A1, SLC44A4, SLC34A2, SLIT2, SPP1, SPRR1B (Sprl), ST6GAL1, ST8SIA1, STAB1, STATE, STEAP, STEAP2, TB4R2, TBX21, TCP1O, TDGF1, TEK, TGFA, TGFB1, TGFB1I1, TGFB2, TGFB3, TGFBI, TGFBR1, TGFBR2, TGFBR3, THIL, THBS1 (thrombospondin-1), THBS2, THBS4, THPO, TIE (Tie-1), TIMP3, a tissue factor, TLR1, TLR2, TLR3, TLR4, TLR5, TLR6, TLR7, TLR8, TLR9, TLR10, TLR11, TNF, TNF-a, TNFAIP2 (B94), TNFAIP3, TNFRSFI1A, TNFRSF1A, TNFRSF1B, TNFRSF21, TNFRSF5, TNFRSF6 (Fas), TNFRSF7, TNFRSF8, TNFRSF9, TNFSF1O (TRAIL), TNFRSF10A, TNFRSF10B, TNFRSF12A, TNFRSF17, TNFSF1 1 (TRANCE), TNFSF12 (APO3L), TNFSF13 (April), TNFSF13B, TNFSF14 (HVEM-L), TNFRSF14 (HVEM), TNFSF15 (VEGI), TNFSF18, TNFSF4 (OX40 ligand), TNFSF5 (CD40 ligand), TNFSF6 (FasL), TNFSF7 (CD27 ligand), TNFSF8 (CD30 ligand), TNFSF9 (4-1BB ligand), TOLLIP, a Toll-like receptor, TOP2A (topoisomerase Iia), TP53, TPM1, TPM2, TRADD, TRAF1, TRAF2, TRAF3, TRAF4, TRAF5, TRAF6, TRKA, TREM1, TREM2, TROP2, TRPC6, TSLP, TWEAK, tyrosinase, uPAR, VEGF, VEGFB, VEGFC, versican, VHL C5, VLA-4, WT1, Wnt-1, XCL1 (lymphotaxime), XCL2 (SCM-Ib), XCRI (GPR5/CCXCR1), YY1, ZFPM2, CLEC4C (BDCA-2, DLEC, CD303, CDH6, CLECSF7), CLEC4D (MCL, CLECSF8), CLEC4E (Mincle), CLEC6A (dectin-2), CLEC5A (MDL-1, CLECSF5), CLEC1B (CLEC-2), CLEC9A (DNGR-1), CLEC7A (dectin-1), CLEC11A, PDGFRa, SLAMF7, GP6 (GPVI), LILRA1 (CD85I), LILRA2 (CD85H, ILT1), LILRA4 (CD85G, ILT7), LILRA5 (CD85F, ILT11), LILRA6 (CD85b, ILT8), LILRB1, NCR1 (CD335, LY94, NKp46), NCR3 (CD335, LY94, NKp46), NCR3 (CD337, NKp30), OSCAR, TARM1, CD30, CD300C, CD300E, CD300LB (CD300B), CD300LD (CD300D), KIR2DL4 (CD158D), KIR2DS, KLRC2 (CD159C, NKG2C), KLRK1 (CD314, NKG2D), NCR2 (CD336, NKp44), PILRB, SIGLEC1 (CD169, SN), SIGLEC5, SIGLEC6, SIGLEC7, SIGLEC8, SIGLEC9, SIGLEC10, SIGLEC11, SIGLEC12, SIGLEC14, SIGLEC15 (CD33L3), SIGLEC16, SIRPA, SIRPB1 (CD172B), TREM1 (CD354), TREM2, KLRF1 (NKp80), 17-1A, SLAM7, MSLN, CTAG1B/NY-ESO-1, MAGEA3/A6, ATP5I (Q06185), OAT (P29758), AIFM1 (Q9Z0X1), AOFA (Q64133), MTDC (P18155), CMC1 (Q8BH59), PREP (Q8K411), YMEL1 (O88967), LPPRC (Q6PB66), LONM (Q8CGK3), ACON (Q99KI0), ODO1 (Q60597), IDHP (P54071), ALDH2 (P47738), ATPB (P56480), AATM (P05202), TMM93 (Q9CQW0), ERGI3 (Q9CQE7), RTN4 (Q99P72), CL041 (Q8BQR4), ERLN2 (Q8BFZ9), TERA (Q01853), DAD1 (P61804), CALX (P35564), CALU (035887), VAPA (Q9WV55), MOGS (Q80UM7), GANAB (Q8BHN3), ERO1A (Q8R180), UGGG1 (Q6P5E4), P4HA1 (Q60715), HYEP (Q9D379), CALR (P14211), AT2A2 (055143), PDIA4 (P08003), PDIA1 (P09103), PDIA3 (P27773), PDIA6 (Q922R8), CLH (Q68FD5), PPIB (P24369), TCPG (P80318), MOT4 (P57787), NICA (P57716), BASI (P18572), VAPA (Q9WV55), ENV2 (P11370), VAT1 (Q62465), 4F2 (P10852), ENOA (P17182), ILK (055222), GPNMB (Q99P91), ENV1 (P10404), ERO1A (Q8R180), CLH (Q68FD5), DSG1A (Q61495), AT1A1 (Q8VDN2), HYOU1 (Q9JKR6), TRAP1 (Q9CQN1), GRP75 (P38647), ENPL (P08113), CH60 (P63038), or CH10 (Q64433), but the present invention is not limited thereto.

The target antigen may be an antigen that is distributed at least 10 times more abundantly in cancer cells that in normal cells.

### [Conjugation Methods]

ADCs released so far are heterogeneous mixtures, with multiple drug-binding sites on the antibody and irregular numbers of bound drugs. The location at which the drug-linker complex is conjugated to the antibody affects the stability and pharmacokinetics-pharmacodynamic properties of the drug. During the ADC manufacturing process, it was difficult to control the DAR, which is the ratio at which the drug is attached to the antibody. When the DAR is high, clearance from plasma is faster, and when the DAR is low, the therapeutic effect is low. In addition, in ADCs with heterogeneous DARs, antibodies with a low DAR compete with antibodies with a high DAR after administration, reducing the effect of the ADC. To solve this problem, a site-specific binding method has been developed.

With the site-specific method, the structure and homogeneity of ADCs may be maintained, the drug may selectively bind to a specific site of the antibody, and the number of the drugs bound may be strictly controlled. There are two main site-specific binding methods, including a method using a native antibody and a method using an engineered antibody.

### 1. Site-Specific Bioconjugation Method by Modifying Native Antibody

Using native antibodies is a convenient method that can avoid the complex mutant antibody selection process or culture optimization process occurring when producing artificial antibodies. Conjugation is performed at an endogenous lysine, histidine, tyrosine, or cysteine residue in the antibody. All ADCs approved through 2021 used these endogenous amino acid residues for conjugation. In a post-translation process, a native antibody in which glycan is incorporated into the Fc region was also used. Since IgG is originally a glycoprotein, N-glycan is present at position N-297 of each heavy chain. A linker-drug complex may be conjugated to this glycosyl site.

In traditional methods, drugs were bonded to the lysine residue and the cysteine residue of antibodies. Since an antibody contains lysine residues at multiple sites, it is difficult to avoid heterogeneity when the method uses lysine. Recently, most ADCs use interchain disulfide cysteine for conjugation. In the case of cysteine conjugation, since the number of cysteines in the antibody is small, not only homogeneity was improved but also the DAR was easily controlled. In the case of lysine and histidine residues, to overcome the heterogeneity problem, as a chemical method, a method of ensuring site-specificity by modifying only a specific lysine or histidine residue in an antibody was also developed.

### 2. Site-Specific Bioconjugation Method Using Engineered Antibody

Since engineered antibodies make it easy to handle the DAR, more homogeneous ADCs are easily made by using this method. Pharmacokinetics-pharmacodynamic properties may be improved by incorporating a native or artificial amino acid residue at a specific site of an antibody.

First, there is an enzymatic method that allows a drug to be conjugated highly selectively using an amino acid tag incorporated into an antibody in a genetic engineering manner. This tag is specifically recognized by an enzyme such as formylglycine-generating enzyme (FGE), microbial transglutaminase (MTG), sortase, or tyrosinase, enabling site-specific conjugation. In the method called SMARTag®, an aldehyde tag was attached for site-specific conjugation. The aldehyde, formylglycine, is attached to a cysteine at a specific site of a monoclonal antibody for conjugation.

Second, there is a cysteine engineering method, and Thiomab® technology uses an engineered cysteine that does not participate in disulfide bonds to bind site-specifically and ensure homogeneity. The Thiomab® technique was first used in anti-MUC16 monoclonal antibodies, where alanine at position 116 in the heavy chain was replaced with a cysteine residue through genetic engineering.

Still another method is site-specific conjugation by incorporating a non-canonical amino acid into an antibody. The incorporated artificial amino acid has a unique chemical structure to allow a drug-linker complex to be conjugated selectively. Non-canonical, artificial amino acids may cause immunogenicity, so caution is required, and for example, cyclopropene derivatives of lysine, and selenocysteine and the like were used.

In one embodiment of the present invention, to provide an ADC in which two types of drug-linker conjugates are homogeneously and symmetrically connected to one antibody, (1) a disulfide (—S—S—) present in the antibody may be reduced to form two thiols (—SH), and by utilizing the resulting thiol sites as attachment sites, a site-specific ADC with a DAR of 4 to 8 may be prepared without separate antibody engineering (Steps 1 and 2 of Examples 4 to 7); and (2) according to the site-specific conjugation method for ADCs developed by Ajinomoto, a peptide reagent having an S—S bond may be used to create a site-specific site in the Fc region of the antibody, and only a free thiol released from the peptide reagent after disulfide bond reduction-partial oxidation may remain in a —SH form, to which a desired drug-linker conjugate may then be additionally bound (Step 3 of Examples 2 to 5, and FIGS. 21A to 21C).

The site-specific conjugation method for ADCs developed by Ajinomoto is for site-specific conjugation at a free-thiol-attached moiety, which is created by acylation of the lysine site at position 248 of the heavy chain of an antibody using an Fc-binding peptide (Peptide Reagent 1 of Scheme 2 in ACS Omega (2019) Vol. 4, pp. 20564-20570). Compared to the method for site-specific conjugation using existing antibody engineering, this method has the advantage that site-specific, homogeneous ADCs can be produced in high yield without antibody engineering.

For example, in the present invention, all four interchain disulfide bonds of an antibody with an IgG structure may be reduced and cleaved to induce 8 free —SH functional groups, and the lysine residue at position 248 of the Fc region of an intermediate-ADC compound with DAR 8 or DAR 4, prepared by reacting all of the resulting —SH functional groups with a linker-payload compound may be allowed to react with the linker-payload compound again, thereby preparing an ADC having a Product 1 or Product 2 structure in FIGS. 18A to 18C (for Product 1, eight payload As introduced by —SH and two payload Bs introduced at Lys 248; for Product 2, four payload As introduced by * structure and two payload Bs introduced at Lys 248) (Examples 2 to 5).

The ADC having the Product 1 or Product 2 structure has a homogeneous structure with two types of payloads in a predetermined ratio, and is able to achieve various pharmacological effects by freely introducing two types or more payloads.

In addition, the use of the same type of payload as payloads A and B can produce ADCs with a DAR value, such as DAR 6 or 10, which is difficult to obtain homogeneously using the existing ADC production method. Finally, various targeting moieties (an aptamer, scFv, a nanobody, a repebody, and a small molecule ligand), rather than a chemical agent having pharmacological efficacy may be used as payload A or B, thereby obtaining ADCs having bi-specific or bi-paratropic characteristics with a predetermined structure. This case is included in the scope of the present invention as well.

### [Pharmaceutically Acceptable Salts]

In this specification, pharmaceutically acceptable salts refer to salts conventionally used in the pharmaceutical industry, including, for example, salts of inorganic ions such as sodium, potassium, calcium, magnesium, lithium, copper, manganese, zinc, and iron, salts of inorganic acids such as hydrochloric acid, phosphoric acid, and sulfuric acid, salts of organic acids such as ascorbic acid, citric acid, tartaric acid, lactic acid, maleic acid, malonic acid, fumaric acid, glycolic acid, succinic acid, propionic acid, acetic acid, orotic acid, and acetylsalicylic acid, and salts of amino acids such as lysine, arginine, and guanine. In addition, pharmaceutically acceptable salts also include salts of organic ions such as tetramethyl ammonium, tetraethyl ammonium, tetrapropyl ammonium, tetrabutyl ammonium, benzyl trimethyl ammonium, and benzetonium, which can be used in pharmaceutical reactions, purification, and separation processes. However, the type of salt referred to in the present invention is not limited by the salts listed above.

### [Various Carrier-Drug Conjugates]

The dual drug-linker conjugate selected according to the present invention can be applied to various types of drug carriers, other than antibodies, and can be widely utilized for various uses by using carriers that can penetrate deep into cancer tissue and facilitate CMC, unlike antibodies.

Carriers may include an antigen-binding site of an antibody, a peptide, a repebody, and/or an aptamer.

| | ADC | PDC | ApDC | Repebody |
|---|---|---|---|---|
| Size | >150 kDa | 2-5 kDa | >20 kDa | 30~50 kDa |
| Advan- | next- | high tissue | high tissue | relatively low |
| tages | generation | permeability | permeability | immuno- |
| | anticancer | low | low | genicity |
| | agent that is | immuno- | immuno- | relatively easy |
| | gaining | genicity | genicity | to control DAR |
| | attention in | able to | able to | high structural |
| | the global | precisely | precisely | stability at high |
| | marker as it | control DAR | control DAR | temperature |
| | can perform | able to | easy | and pH |
| | two | introduce | chemical | |
| | functions | modified | modification | |
| | simultaneously: | amino acid | | |
| | target | such as | | |
| | selectivity of | non-natural | | |
| | antibody and | amino acid | | |
| | cytotoxicity of | | | |
| | drug | | | |
| Disadvan- | high | low in vivo | low in vivo | in the early |
| tages | immuno- | stability | stability | state of |
| | genicity | | | development, |
| | difficulty in | | | related research |
| | DAR control | | | and |
| | low tissue | | | optimization |
| | permeability | | | technology are |
| | | | | required |

Aptamer-drug conjugates (ApDCs) utilize an aptamer instead of an antibody of the ADC. Aptamers are single-stranded nucleic acids with a three-dimensional structure. They are discovered through the systematic evolution of ligands by exponential enrichment (SELEX) process. SELEX is a technology that obtains a functional nucleic acid binding to target protein molecules in a compound library.

Aptamers are also called chemical antibodies because they can very strongly and selectively bind to a target. Aptamers are about 20 kDa in in size and are known to have better cellular penetration and lower immunogenicity than antibodies.

Since aptamers can be chemically synthesized, precise design of the conjugation sites and number of conjugated drugs is possible in the preparation of ApDCs. ApDCs have lower production costs than ADCs.

Aptamers are typically consisting of natural nucleic acids, which are degraded by nucleases in the body, making them less stable in vivo. However, the easy chemical modification of aptamers can be leveraged to overcome limitations in the stability of modified aptamers.

Peptide-drug conjugates (PDCs) are a form of an ADC in which a peptide is introduced instead of an antibody. Peptides consist of amino acids and have a size of 500 to 5000 Da. Peptides are very small compared to antibodies of 150 kDa or more. Accordingly, peptide-based PDCs have superior cell penetration capabilities compared to ADCs and are much less likely to develop immunogenicity. In addition, peptides can be chemical synthesized. To this end, PDCs may not only have a very low production cost, but also allow precise regulation of the conjugation location and ratio of the peptide and the drug.

Generally, peptides are easily degraded by proteases and therefore have a short biological half-life. To overcome this limitation of peptide-based drug conjugates, strategies have been proposed to utilize modified peptides, such as to introduce cyclic peptides or non-natural amino acids.

A repebody is a type of artificial antibody that does not have an antibody skeleton but has the function of recognizing antigens, like an antibody. A repebody specific for a target protein may be discovered using phage display technology.

Phage display is a technique by which a desired protein is expressed on the surface of a bacteriophage. A repebody is about 30 kDa in size, which is about 20% of an antibody drug. Therefore, repebodies are known to have relatively low immunogenicity and improved cell penetration compared to antibodies. In addition, it is expected that the thermal pH stability of a repebody can be regulated to increase structural stability. Compared to antibodies, repebodies are also considered to have relatively low production costs. Due to these advantages of repebodies, interest in the development of a repebody-drug conjugate (repebody-DC) as a strategy to replace an antibody with a repebody is also increasing.

### [Pharmaceutical Composition for Preventing or Treating Cancer]

A pharmaceutical composition for preventing or treating cancer is provided, which includes the ADC or a pharmaceutically acceptable salt thereof according to the present invention described above as an active ingredient.

In addition, according to one embodiment of the present invention, a method of treating or preventing cancer is provided, which includes administering a therapeutically effective amount of the ADC into a subject in need thereof. The subject is a mammal, such as a human.

Since the ADC of the present invention specifically binds to an antigen of cancer cells and releases the drug within or outside the cancer cells to exhibit cytotoxicity, it may be effectively used in cancer treatment or prevention. The anticancer activity of the ADC of the present invention is described above.

In the present invention, the cancer may be solid cancer or blood cancer. For example, the cancer may be one or more selected from the group consisting of pseudomyxoma, intrahepatic cholangiocarcinoma, hepatoblastoma, liver cancer, thyroid cancer, colon cancer, testicular cancer, myelodysplastic syndrome, glioblastoma, oral cancer, lip cancer, mycosis fungiodes, acute myeloid leukemia, acute lymphoblastic leukemia, basal cell carcinoma, ovarian epithelial carcinoma, ovarian germ cell carcinoma, male breast cancer, brain cancer, pituitary adenoma, multiple myeloma, gallbladder cancer, bile duct cancer, colorectal cancer, chronic myelogenous leukemia, chronic lymphocytic leukemia, retinoblastoma, choroidal melanoma, ampulla of Vater cancer, bladder cancer, peritoneal cancer, parathyroid cancer, adrenal cancer, paranasal sinus cancer, non-small cell lung cancer, tongue cancer, astrocytoma, small cell lung cancer, pediatric brain cancer, pediatric lymphoma, childhood leukemia, childhood leukemia, small bowel cancer, meningioma, esophageal cancer, glioma, renal pelvis cancer, kidney cancer, heart cancer, duodenal cancer, malignant soft tissue cancer, malignant bone cancer, malignant lymphoma, malignant mesothelioma, malignant melanoma, eye cancer, vulvar cancer, ureteral cancer, urethral cancer, unknown primary neoplasm, lymphoma, gastric cancer, gastric carcinoma, gastrointestinal stromal cancer, Wilms' cancer, breast cancer, sarcoma, penile cancer, pharyngeal cancer, gestational trophoblastic disease, cervical cancer, endometrial cancer, uterine sarcoma, prostate cancer, metastatic bone cancer, metastatic brain cancer, mediastinal cancer, rectal cancer, occipital carcinoid tumors, vaginal cancer, spinal cancer, vestibular schwannoma, pancreatic cancer, salivary gland cancer, Kaposi's sarcoma, Paget's disease, tonsil cancer, squamous cell carcinoma, lung adenocarcinoma, lung cancer, squamous cell lung cancer, skin cancer, anal cancer, rhabdomyosarcoma, laryngeal cancer, pleural cancer, blood cancer, and thymic carcinoma, but the present invention is not limited thereto. In addition, the caner includes not only primary cancer but also metastatic cancer.

The term “therapeutically effective amount” used herein refers to an amount of the immunoconjugate effective in the treatment or prevention of cancer. Specifically, the “therapeutically effective amount” refers to an amount sufficient for treating a disease at a reasonable benefit/risk ratio applicable for medical treatment, and an effective dose may be determined by parameters including the type of a subject, the severity of a disease, the subject's age and sex, the type of a disease, drug activity, the sensitivity to a drug, administration time, an administration route and an excretion rate, the duration of treatment and drugs simultaneously used, and other parameters well known in the medical field. The pharmaceutical composition of the present invention may be administered separately or in combination with other therapeutic agents, and may be sequentially or simultaneously administered with a conventional therapeutic agent, or administered in a single or multiple dose(s). In consideration of all of the above-mentioned parameters, it is important to achieve the maximum effect with the minimum dose without side effects. As the immunoconjugate of the present invention exhibits a dose-dependent effect, the administration dose may be easily determined by various factors such as a patient's condition, age, sex, and complications. The active ingredient of the pharmaceutical composition of the present invention has excellent safety, so it may be used at a dose exceeding the determined administration dose.

In addition, according to one embodiment of the present invention, a use of the immunoconjugate for producing a medicament for treatment or prevention of cancer is provided. The immunoconjugate for producing medicaments may be mixed with acceptable additives, diluents, and carriers, and may be prepared as a composite preparation along with other active agents to exhibit a synergistic effect of the active ingredients.

The details on the use, composition, and treatment method of the present invention are equally applied unless they are contradictory.

### Advantageous Effects

According to the present invention, an ADC in which two types of drug-linker conjugates are connected to one antibody using a linker can overcome the difficulties of initially approved ADCs in which toxicity is increased when the anticancer effect is increased, and conversely, when safety is increased, the anticancer effect is not fully exerted, thereby narrowing the therapeutic window, that is, it can expand the therapeutic window of ADC drugs and increase a tumor response rate.

The ADC provided in the present invention can selectively deliver a potent cytotoxic drug that kills cancer cells even at a pM level concentration only to cancer tissue and minimize non-selective uptake, ensuring both anticancer efficacy and safety.

### DESCRIPTION OF DRAWINGS

FIG. 1 shows the results of comparative analysis of the IC₅₀ of Trastuzumab-MMAE(2)-25-6(6) in a Her2 positive cell line, MDA-MB-453, and a Her2 negative cell line, MDA-MB-468.

FIG. 2 shows the results of comparative analysis of the IC₅₀ of Trastuzumab-veliparib(4)-25-6(4) in a Her2 positive cell line, MDA-MB-453, and a Her2 negative cell line, MDA-MB-468.

FIG. 3 shows the synthetic design concept of a novel active camptothecin derivative (a) having a dual mechanism of action (MoA) that degrades the oncoprotein DDX5 along with the ability to inhibit topoisomerase-1 through an improved FL118 structure.

FIG. 4 illustrates the structural formula of camptothecin (CPT) and its binding with topoisomerase-1, which expands the molecular design concept of FL118 and PBX-7011 from camptothecin, the derivation of compounds PBX-7014 and PBX-7016 having novel structures expanded from PBX-7011, and the results of calculating their hydrophilicity.

FIG. 5 shows the mechanism of ADC toxicity (refer to Cancers 2023, 15(3), 713; https://doi.org/10.3390/cancers15030713).

FIG. 6 is a conceptual diagram showing the MoA of an ADC that includes a drug-linker conjugate consisting of the combination of a camptothecin-based drug and an acid-sensitive linker.

FIG. 7 shows the analysis results confirming that the DAR of an ADC (PBX-001) synthesized in Preparation Example 8 is 8.

FIG. 8 shows the antigen binding assay results of PBX-001.

FIG. 9 is a stability graph according to pH, which compares the payload release degrees of PBX-001 and Trodelvy according to pH.

FIG. 10 is a serum stability graph that compares PBX-001 and Trodelvy.

FIG. 11 is a graph showing that PBX-001 is effective on drug-resistant cells expressing the ABCG2 transporter proteing even at a low concentration, compared to Trodelvy.

FIG. 12 is a result showing that PBX-001 has tumor growth inhibition (TGI) at a low concentration, compared to Trodelvy.

FIG. 13 is a result showing the TGI (in vitro and in vivo efficacy) of anti-HER2 ADC-FL118.

FIG. 14 is a result showing the TGI (in vitro and in vivo efficacy) of anti-EGFR ADC-FL118.

FIG. 15 is a result showing the excellent anticancer effect of PBX-001, compared to Trodelvy.

FIG. 16 shows the non-clinical safety profile of PBX-001, compared to Trodelvy.

FIG. 17 is a Western blotting result that shows the degree of inhibition of anti-apoptotic proteins, showing that the FL118 drug and exatecan drug exhibit a better effect in downregulating a tumor protein in HCT-8 and FaDu cell lines, compared to the SN-38 drug.

FIG. 18 is a result showing that FL118 drug exhibits better in vitro cytotoxicity.

FIG. 19 illustrates the MoAs of various self-immolative spacers.

FIG. 20 shows drug-linker conjugates, which are divided into four groups (A, B, C, and D) according to payload cytotoxicity and linker stability.

FIGS. 21A to 21C are schematic diagrams conceptualizing each step of a method of preparing an antibody-drug conjugate (ADC) in which two types of drug-linker conjugates are homogeneously and symmetrically connected to one antibody according to one embodiment of the present invention.

FIG. 22 illustrates Step 3 using a peptide reagent to introduce two MMAEs in Examples 2 to 5.

FIG. 23 shows the SEC chromatogram (left) when CL2A-FL118 is added and reacted without removing residual vc-mc-PAB-MMAE after the first reaction in Example 1, and the SEC chromatogram (right) when CL2A-FL118 is added and reacted after PD-10 removal.

FIG. 24 shows the results of SES-PAGE after conjugation in Example 1.

FIG. 25 shows the results of LC-MS (molecular weight analysis) of the light chain portion of an ADC prepared without mc-vc-PAB-MMAE removal after the first reaction in Example 1 (FIG. 25A); the results of LC-MS (molecular weight analysis) of the light chain portion of an ADC prepared with mc-vc-PAB-MMAE removal after the first reaction (FIG. 25B); the results of LC-MS (molecular weight analysis) of the heavy chain portion of an ADC prepared without mc-vc-PAB-MMAE removal after the first reaction (FIG. 25C); and the results of LC-MS (molecular weight analysis) of the heavy chain portion of ADC prepared with mc-vc-PAB-MMAE removal after the first reaction (FIG. 25D).

FIGS. 26 and 27 show the results of Western blot results that show the presence or absence/degree of DDX5 and p-DDX5 protein degradation of various camptothecin-based drugs (FL118 drug, SN-38 drug, Exatecan drug, PBX-7011, PBX-7014, and PBX-7016) in a FaDu cell line and an A549 cell line, and the degree of inhibition of various anti-apoptotic proteins as well. FIGS. 5 and 7 are graphic representations of the concentrations of the Western blot results (FIGS. 4 and 6) performed on the FaDu cell line and the A549 cell line.

FIG. 28 shows the results of in vitro cell viability comparison evaluation for various camptothecin-based drugs in a FaDu cell line or A549 cell line.

FIG. 29 shows the results of in vitro cell viability comparison evaluation for various camptothecin-based drugs in an MDA-MB-453 (HER2++) cell line and a FaDu (HER2+) cell line.

FIG. 30 shows the results of in vitro cell viability comparison evaluation for various camptothecin-based drugs and ADCs using them as payloads in an MDA-MB-453 (HER2++) cell line, a FaDu (HER2+) cell line, and an MDA-MB-468(HER2−) cell line.

FIG. 31 shows the Western blotting results of Tra-CL2A-FL118/Tra-CL2A-Exatecan.

FIGS. 32 and 33 show the results of in vitro cell viability comparison evaluation for various camptothecin-based drugs and ADCs using them as payloads, such as Tra-CL2A-FL118/Tra-CL2A-Exatecan in each of an MDA-MB-453(HER2++) cell line, and an SK-BR-3 cell line.

FIG. 34 shows the results of counting cell numbers through FACS based on the presence or absence of GFP expression in an ADC having a camptothecin-based drug as a payload after co-culturing an MDA-MB-453 (HER2++) cell line and a GFP-expressed MDA-MB-468 (HER2−) cell line.

FIG. 35 shows the results of counting cell numbers through FACS using a HER2-FITC antibody based on the presence or absence of HER2 expression in an ADC having a camptothecin-based drug as a payload after co-culturing an MDA-MB-453 (HER2++) cell line and an MDA-MB-468(HER2−) cell line.

FIG. 36 shows the comparison of the Log Ps, c Log Ps, and topological polar surface areas (tPSAs) of FL118, exatecan, SN-38, Dxd, compound A (PBX-7011), compound B (PBX-7012), compound C (PBX-7014), compound D (PBX-7015), compound E (PBX-7016), and compound F (PBX-7017).

FIG. 37 shows the comparison of the Log Ps, c Log Ps, and tPSAs of various camptothecin-based drugs, compound G, compound H, compound I, compound J, compound G′, compound H′, compound I′, and compound J′, which degrade the DDX5 protein. FIG. 38 shows Scheme 2. Gram-Scale Synthesis of Trastuzumab-AJICAP-MMAE.

### MODES OF THE INVENTION

Hereinafter, the present invention will be described in further detail with reference to examples. However, the following examples are intended only to clearly illustrate the technical features of the present invention and do not limit the scope of the present invention.

### Preparation Example 1: Synthesis of PBX-7011 and PBX-7012

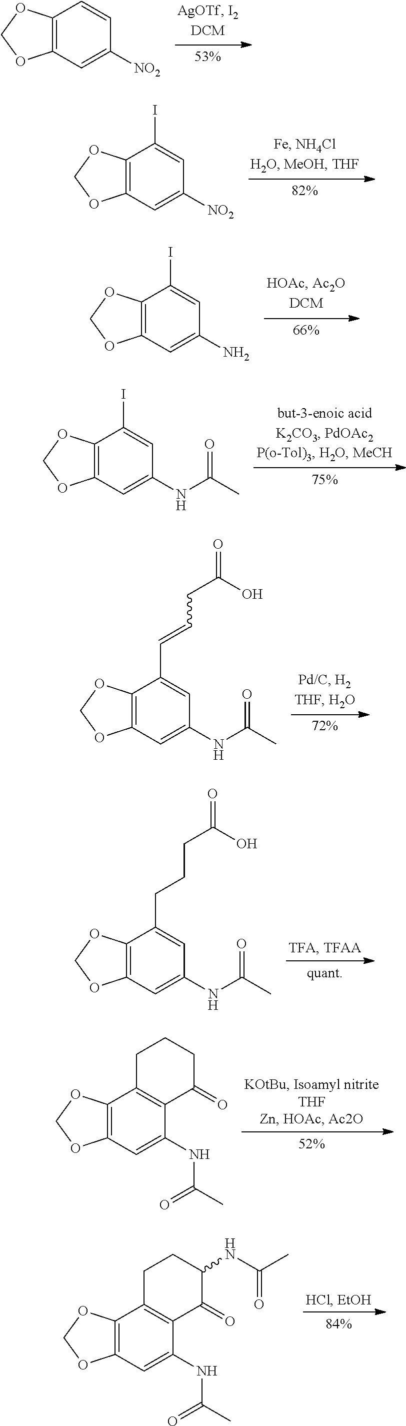

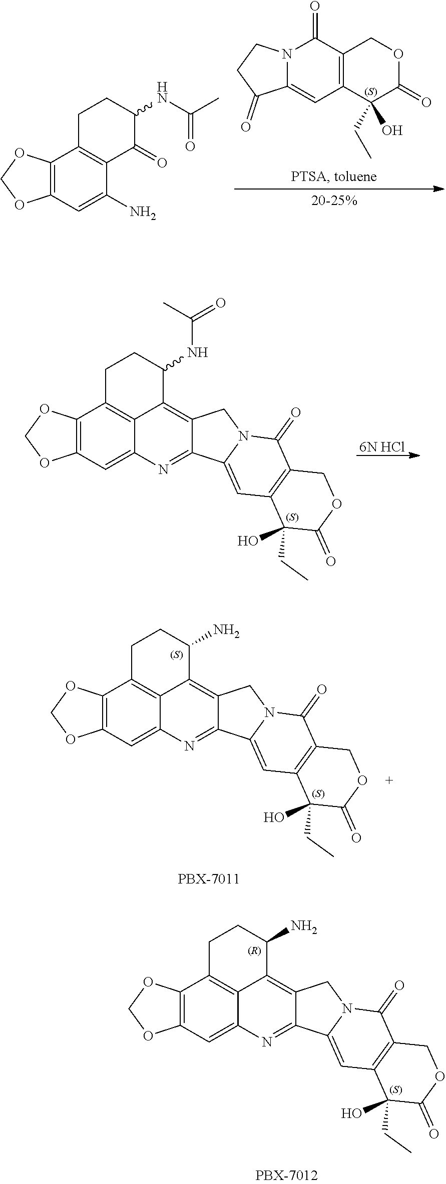

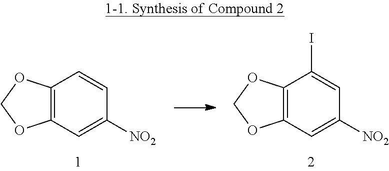 [SMILES: O=[N+]([O-])c1ccc2c(c1)OCO2 ; O=[N+]([O-])c1cc(I)c2c(c1)OCO2]

Silver triflate (57.7 g, 224 mmol) and iodine (57.0 g, 224 mmol) were added to a 5-nitrobenzo[d][1,3]dioxol (25 g, 150 mmol) solution in a dichloromethane (748 mL) flask. The solution was stirred in a room-temperature dark room under an N₂ atmosphere.

AgI was removed by filtration and the solid was washed with dichloromethane (100 mL). The solvent was removed under reduced pressure and the residue was distributed between EtOAc (250 mL) and a 5% (v/v) NH₄OH/H₂O solution (200 mL). An organic layer was separated, washed with 1M Na₂SO₃ (5×200 mL) and brine (200 mL), dried with Na₂SO₄, treated with activated carbon, and filtered through Celite. The solvent was evaporated under reduced pressure to give obtain a crude product as a brown solid. This was triturated in ice-cold EtOH (400 mL) and filtered, and the solid was washed with ice-cold EtOH (100 mL) to obtain a product as a pale brown-gray solid (14.3 g). The solvent was removed from the filtrate, and the crude product was triturated again in ice-cold EtOH (300 mL). The solid was collected by filtration and washed with EtOH (50 mL) to obtain an additional amount of product (10.2 g) as a dark brown-gray solid. Both batches were used as is without additional purification.

SC_ACID: m/z 294.2 [M+H]⁺

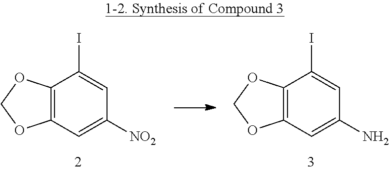 [SMILES: O=[N+]([O-])c1cc(I)c2c(c1)OCO2 ; Nc1cc(I)c2c(c1)OCO2]

Iron powder (6.67 g, 119 mmol) and ammonium chloride (6.39 g, 119 mmol) were added to a suspension of 4-iodo-6-nitrobenzo[d][1,3]dioxol (8.75 g, 29.9 mmol) in a mixture of water (80 mL), methanol (40.0 mL) and tetrahydrofuran (40.0 mL). The suspension was heated to 75° C. and stirred for 16.5 hours. The reaction mixture was cooled to room temperature and concentrated under reduced pressure. The resulting black solid was suspended in ethyl acetate (100 mL) and the solution was filtered. The residue was washed with EtOAc (3×50 mL). The mixture was transferred to a separatory funnel, water was added, and the aqueous layer was removed. The organic layer was washed with a saturated aqueous NaHCO₃ solution (150 mL) and brine (150 mL). The organic layer was dried over Na₂SO₄, and the solvent was removed under reduced pressure to obtain a brown solid (4.75 g, 60% yield). An additional product was recovered by thoroughly washing the iron residue with ethyl acetate. Subsequently, the organic fraction was washed with a saturated aqueous NaHCO₃ solution (150 mL) and brine (150 mL), and dried over Na₂SO₄. The solvent was removed under reduced pressure to obtain a brown solid (1.74 g, 22% yield).

SC_ACID: m/z 264.0 [M+H]⁺

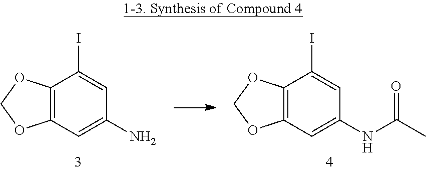

Acetic anhydride (2.7 5 mL, 29.2 mmol) and triethylamine (4.06 mL, 29.2 mmol) were added to a solution of 7-iodobenzo[d][1,3]dioxol-5-amine (6.39 g, 24.29 mmol) in dichloromethane (49 mL). The reaction mixture was stirred at room temperature overnight. After several hours, additional DCM (10 mL) was added. The suspension was filtered through a sintered funnel and washed with ice-cold DCM (10 mL). The product was additionally dried under reduced pressure to obtain a light gray solid (4.46 g). The filtrate was concentrated under reduced pressure, and the residue was dissolved in EtOAc, washed with water and brine, dried over Na₂SO₄ and concentrated under reduced pressure to obtain a brown solid (up to 3 g). The brown solid was purified by flash column chromatography (80 g Si, 0-100% ethyl acetate in heptane). All batches of product were triturated with ice-cold EtOAc (5-10 mL). Two batches were combined and further dried, thereby obtaining an off-white solid.

Total yield: 5.0 g, 66%

SC_ACID: m/z 306.0 [M+H]⁺

¹H NMR (400 MHz, DMSO-d6) δ 9.88 (s, 1H), 7.39 (d, J=1.9 Hz, 1H), 7.17 (d, J=1.9 Hz, 1H), 6.04 (s, 2H), 1.99 (s, 3H).

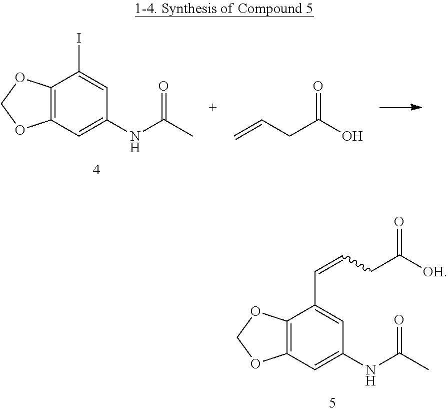

A slurry of N-(7-iodobenzo[d][1,3]dioxol-5-yl)acetamide (5.06 g, 16.59 mmol), but-3-enoic acid (1.69 mL, 19.90 mmol) and potassium carbonate (2.98 g, 21.56 mmol) in acetonitrile (40 mL) was cooled to 0 to 5° C. in a condenser-equipped 3-neck flask. Water (13.33 mL) was slowly added to generate gas. When gas generation stopped, the mixture was degassed with Ar for 30 minutes. Tri-o-tolylphosphine (0.505 g, 1.659 mmol) and palladium acetate (0.186 g, 0.829 mmol) were added, and the mixture was degassed again for 30 minutes and then heated at reflux under Ar. The reaction product was cooled to room temperature and filtered through Celite. The filter cake was washed with H₂O and EtOAc. The organic solvent was removed from the filtrate under vacuum and the aqueous material was acidified with concentrated HCl until pH=1-2. The aqueous layer was extracted with EtOAc, and the combined organic phases were washed with brine, dried over Na₂SO₄, filtered, and concentrated under reduced pressure to obtain a crude product. The crude product was triturated in ice-cold EtOAc (30 mL) and the solid was collected by filtration to give a product as a brown solid. The mother liquid was concentrated under reduced pressure and purified through flash chromatography (80 g Si, 0-100% EtOAc in heptane). The product fraction was concentrated under reduced pressure to obtain a light brown foam. Total yield: 3.46 g, 75%. A mixture of E/Z isomers was obtained.

SC_ACID: m/z 264.4 [M+H]⁺

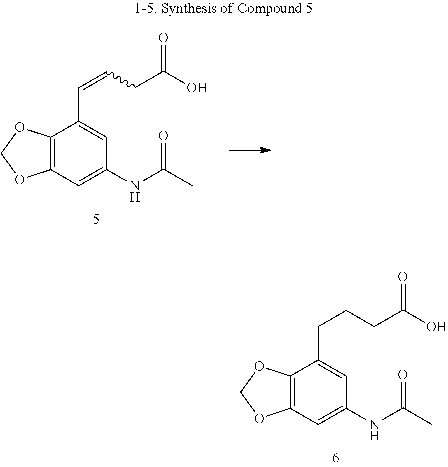 [SMILES: CC(=O)Nc1cc(C=CCC(=O)O)c2c(c1)OCO2 ; CC(=O)Nc1cc(CCCC(=O)O)c2c(c1)OCO2]

A suspension of 4-(6-acetamidobenzo [d][1,3]dioxol-4-yl)but-3-enoic acid (3.47 g, 13.18 mmol) in tetrahydrofuran (50 mL)/water (50 mL) was degassed with N₂ for 15 minutes. Before heating to 40° C. under H₂ (balloon), Pd/C (2.97 g, 1.397 mmol) was added and the suspension was degassed with H₂ for 5 minutes. After 24 hours, nitrogen and additional Pd/C (2.97 g, 1.397 mmol) were added to the reaction mixture. The reaction mixture was bubbled with hydrogen for 10 minutes and stirred overnight at 45° C. under hydrogen. The reaction product was filtered through Celite. A filter cake was washed with H₂O (50 mL), and EtOAc (50 mL) and an organic solvent were removed from the filtrate under vacuum. An aqueous solution was acidified with concentrated HCl until pH=1, and the dark brown/green precipitate was collected by filtration through a sintered funnel. The aqueous filtrate was extracted. The combined organic phases were washed with brine, dried over Na₂SO₄, and filtered, and the solvent was removed under vacuum to obtain a brown oil. Since a product recovery rate was low, a filter cake was washed with EtOAc (50 mL), water (200 mL), and/or OH (400 mL). The water/EtOAc flush was added to a flask containing the solid from an MeOH flush. The aqueous layer was acidified to a thick concentration. HCl and an off-gray precipitate were formed until pH=1, which were collected by filtration through a sintered funnel. After extracting the aqueous filtrate with EtOAc (3×200 mL), LCMS showed that all products were removed from the aqueous phase. The combined organic phases were washed with brine, dried over Na₂SO₄, and filtered, and the solvent was removed under vacuum to obtain a light brown solid. All product batches were combined and purified through flash column chromatography (40 g Si, 0-10% MeOH in DCM). The product fractions were concentrated to obtain a light brown solid. Yield: 2.52 g, 72%.

SC_ACID: 266.2 [M+H]⁺

¹H NMR (400 MHz, DMSO-d6) δ 12.07 (s, 1H), 9.78 (s, 1H), 7.14 (d, J=2.0 Hz, 1H), 6.80 (d, J=2.2 Hz, 1H), 5.95 (s, 2H), 2.50-2.46 (m, 2H), 2.23 (t, J=7.4 Hz, 2H), 1.98 (s, 3H), 1.84-1.70 (m, 2H).

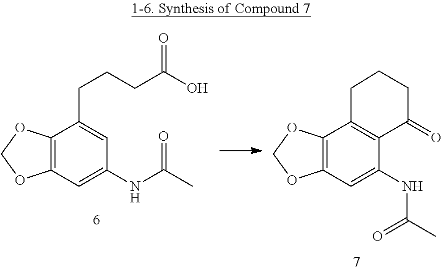 [SMILES: CC(=O)Nc1cc(CCCC(=O)O)c2c(c1)OCO2 ; CC(=O)Nc1cc2c(c3c1C(=O)CCC3)OCO2]

A suspension of 4-(6-acetamidobenzo[d][1,3]dioxol-4-yl)butanoic acid (150 mg, 0.565 mmol) in TFA (433 μL, 5.65 mmol) was cooled on ice. TFAA (157 μL, 1.131 mmol) was added. The mixture was stirred at 0-5° C. for 1 hour, and over time it turned into a dark solution. The reaction mixture was added dropwise to an ice-cold saturated aqueous NaHCO₃ solution (10 mL), and the aqueous solution was extracted with ethyl acetate (3×25 mL). The combined organic layers were dried over sat. NaHCO₃, brine and Na₂SO₄ and filtered, and the solvent was removed under vacuum to obtain a salmon pink solid. Yield: 140 mg, 100%.

SC_ACID: m/z 248.2 [M+H]⁺

¹H NMR (400 MHz, DMSO-d6) δ 12.34 (s, 1H), 8.11 (s, 1H), 6.13 (s, 2H), 2.80 (t, J=6.2 Hz, 2H), 2.66-2.58 (m, 2H), 2.12 (s, 3H), 2.01-1.91 (m, 2H).

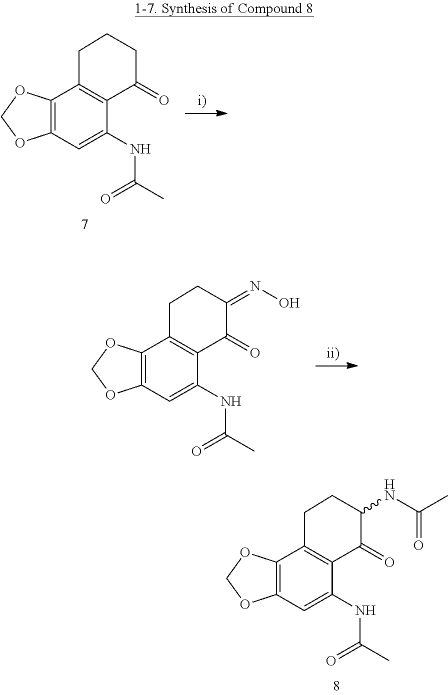 [SMILES: CC(=O)Nc1cc2c(c3c1C(=O)CCC3)OCO2 ; CC(=O)Nc1cc2c(c3c1C(=O)/C(=N\O)CC3)OCO2 ; CC(=O)Nc1cc2c(c3c1C(=O)C(NC(C)=O)CC3)OCO2]

A suspension of N-(6-oxo-6,7,8,9-tetrahydronaphto [1,2-d][1,3]dioxol-5-yl)acetamide (50 mg, 0.202 mmol) in tetrahydrofuran (1.2 mL) was cooled to 0° C., and potassium tert-butoxide (27.2 mg, 0.243 mmol) and isoamyl nitrite (35.0 μL, 0.263 mmol) were added. The dark green mixture was stirred on ice (<5° C.) for 1.5 hours. Acetic acid (170 μL, 2.94 mmol), acetic anhydride (170 μL, 1.810 mmol), and zinc dust (66.1 mg, 1.011 mmol) were added to the reaction mixture. The suspension was stirred at 0° C. for 2 hours. The reaction mixture was filtered over Celite and flushed with DCM. The filtrate was concentrated under reduced pressure to obtain a black oily material. A crude product was purified through flash column chromatography (4 g Si, 04% MeOH in DCM). A product fraction was concentrated to obtain a gray solid (32 mg, 52%) with 80 to 90% purity. A higher purity sample may be obtained by purification using preparative MPLC.

SC_ACID: m/z 305.4 [M+H]⁺

1H NMR (400 MHz, DMSO-d6) δ 12.10 (s, 1H), 8.21 (d, J=8.0 Hz, 1H), 8.12 (s, 1H), 6.15 (d, J=9.3 Hz, 2H), 4.66-4.56 (m, 1H), 2.93 (dd, J=8.9, 4.0 Hz, 2H), 2.14 (s, 3H), 2.13-1.93 (m, 2H), 1.91 (s, 3H).

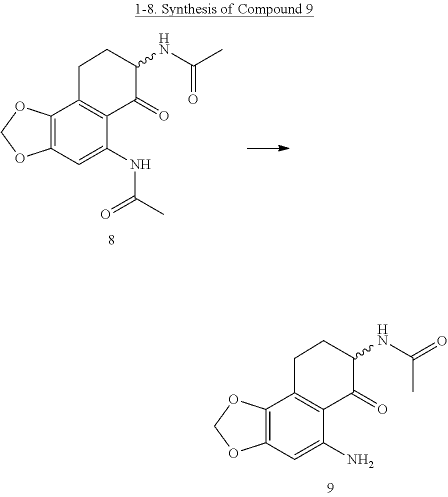 [SMILES: CC(=O)Nc1cc2c(c3c1C(=O)C(NC(C)=O)CC3)OCO2 ; CC(=O)NC1CCc2c3c(cc(N)c2C1=O)OCO3]

N,N′-(6-oxo-6,7,8,9-tetrahydronaphto[1,2-d][1,3]dioxol-5,7-diyl)diacetamide (244 mg, 0.802 mmol) was suspended in 2M hydrochloric acid (4.69 mL, 9.38 mmol) in ethanol/water (5/1). The mixture was heated to 55° C. for 4 hours. The black mixture was cooled to 0 to 5° C. Triethylamine (1.4 mL, 10.04 mmol) was added dropwise while stirring. The mixture was then diluted with EtOH and dried by evaporation. The residue was distributed between water and DCM. The layers were separated and the aqueous layer was extracted once with DCM. The combined organic layers were dried over Na₂SO₄ and concentrated to obtain a product as a brown solid (178 mg, 73%) with 86% purity.

SC_ACID: m/z 263.0 [M+H]⁺

1H NMR (400 MHz, DMSO) δ 8.04 (d, J=8.0 Hz, 1H), 6.20 (s, 1H), 5.94 (d, J=6.0 Hz, 2H), 4.48-4.41 (m, 1H), 3.08 (s, 2H), 2.88-2.69 (m, 2H), 2.15-2.03 (m, 1H), 1.88 (s, 3H), 1.86-1.75 (m, 1H).

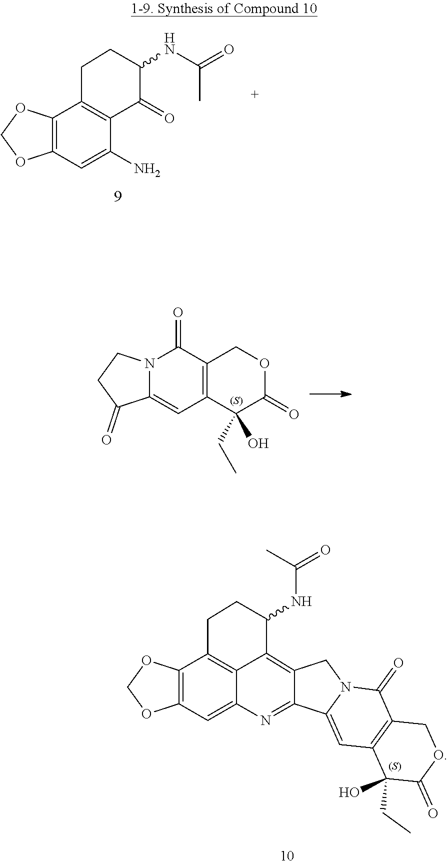

(4S)-4-ethyl-7,8-dihydro-4-hydroxy-1H-pyrano[3,4-f]indolizine-3,6,10(4H)-trione (146 mg, 0.555 mmol) and N-(5-amino-6-oxo-6,7,8,9-tetrahydronaphto[1,2-d][1,3]dioxol-7-yl)acetamide (112 mg, 0.427 mmol) were added to dry toluene (4.5 mL). PPTS (21 mg, 0.085 mmol) was added, and the reaction mixture was stirred at 115° C. for 40 hours.

The reaction mixture was cooled to room temperature. The suspension was diluted with 2 mL of DCM and filtered. A black residue (210 mg) was obtained.

The crude product was purified by column chromatography (12 g Si, 0 to 7% methanol in DCM) to obtain a product (45 mg, 21%) as a brown solid.

LCMS analysis showed two diastereomers.

SC_ACID: m/z 490.2 [M+H]⁺

¹H NMR (400 MHz, DMSO-d6) δ 8.47 (t, J=9.3 Hz, 1H), 7.42 (s, 1H), 7.24 (s, 1H), 6.49 (s, 1H), 6.29 (d, J=5.2 Hz, 2H), 5.57-5.49 (m, 1H), 5.41 (s, 2H), 5.23-5.07 (m, 2H), 3.09-3.00 (m, 2H), 2.11-2.01 (m, 2H), 1.91 (s, 3H), 1.89-1.79 (m, 2H), 0.87 (t, J=7.1 Hz, 3H).

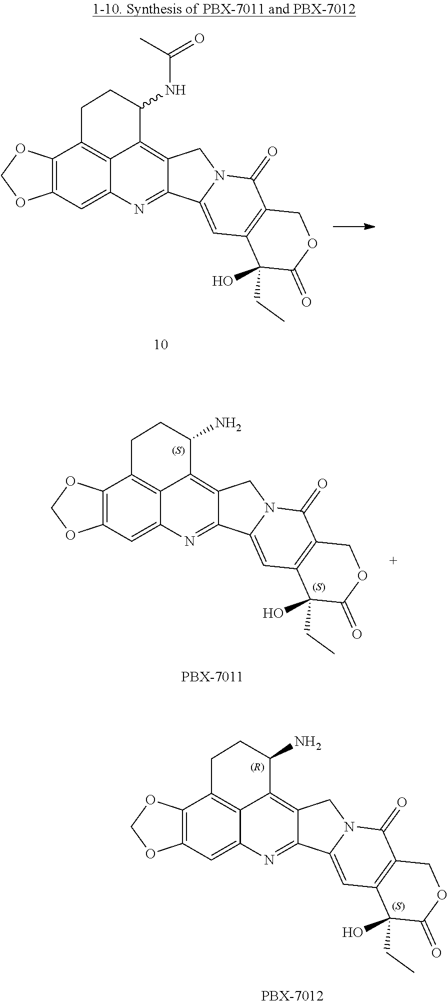

N-((10S)-10-ethyl-10-hydroxy-11,14-dioxo-2,3,10,11,14,16-hexahydro-1H,13H-benzo[de][1,3]dioxolo[4,5-g]pyrano[3′,4′:6,7]indolizino[1,2-b]quinolin-1-yl)acetamide (73.5 mg, 0.150 mmol) was dissolved in 1.0 mL of 6N HCl, and stirred at 90° C. for 8 hours and then at room temperature overnight. The reaction mixture was concentrated under reduced pressure and purified twice through acidic preparative MPLC (Luna2-30) twice. A fraction containing the separated diastereomer was acidified with 5 drops of 3N HCl and lyophilized to obtain a product as a yellow solid.

1^{st} eluting isomer: PBX-7011, 23 mg, 34% yield

U_AN_ACID: m/z 448.4 [M+H]⁺

¹H NMR (400 MHz, DMSO-d6) δ 8.54 (s, 3H), 7.50 (s, 1H), 7.27 (s, 1H), 6.52 (s, 1H), 6.34 (d, J=13.3 Hz, 2H), 5.77 (d, J=19.3 Hz, 1H), 5.44 (s, 2H), 5.37 (d, J=19.3 Hz, 1H), 5.05 (s, 1H), 3.19-3.02 (m, 2H), 2.46 (s, 1H), 2.20-2.03 (m, 1H), 1.94-1.81 (m, 2H), 0.88 (t, J=7.3 Hz, 3H).

2^{nd} eluting isomer: PBX-7012, 28 mg, 41% yield

U_AN_ACID: m/z 448.2 [M+H]⁺

¹H NMR (400 MHz, DMSO-d6) δ 8.57 (d, J=4.7 Hz, 3H), 7.51 (s, 1H), 7.27 (s, 1H), 6.52 (s, 1H), 6.34 (d, J=12.2 Hz, 2H), 5.76 (d, J=19.4 Hz, 1H), 5.44 (s, 2H), 5.37 (d, J=19.4 Hz, 1H), 5.08 (s, 1H), 3.16-2.98 (m, 2H), 2.46 (s, 1H), 2.18-2.06 (m, 1H), 1.94-1.80 (m, 2H), 0.87 (t, J=7.3 Hz, 3H).

### Preparation Example 2: Synthesis of PBX-7014 and PBX-7015

This example illustrates the synthesis of Compounds PBX-7014 and PBX-7015, starting from two separated diastereomers of the exatecan-hybrid compounds (PBX-7011 and PBX-7012).

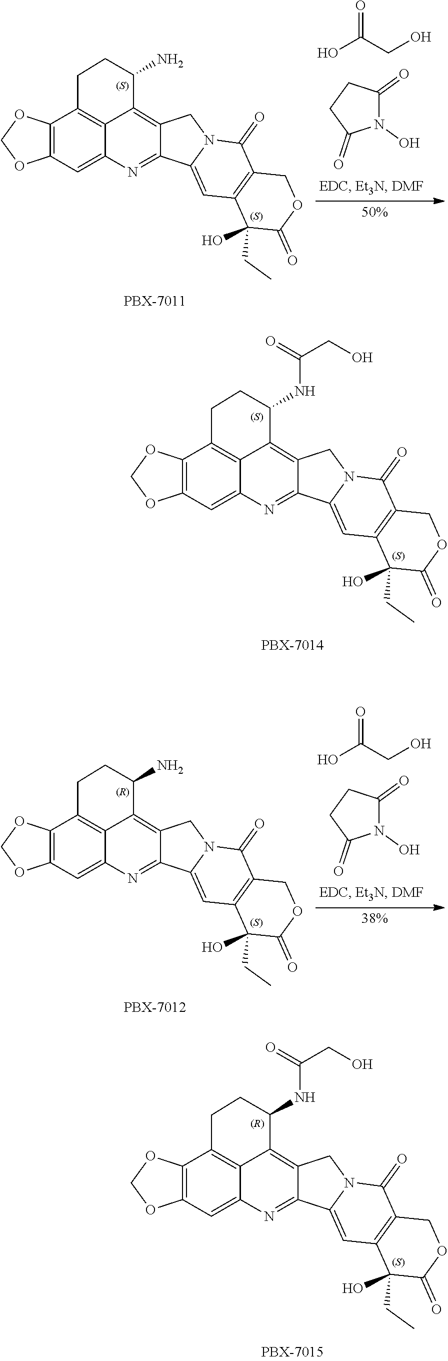

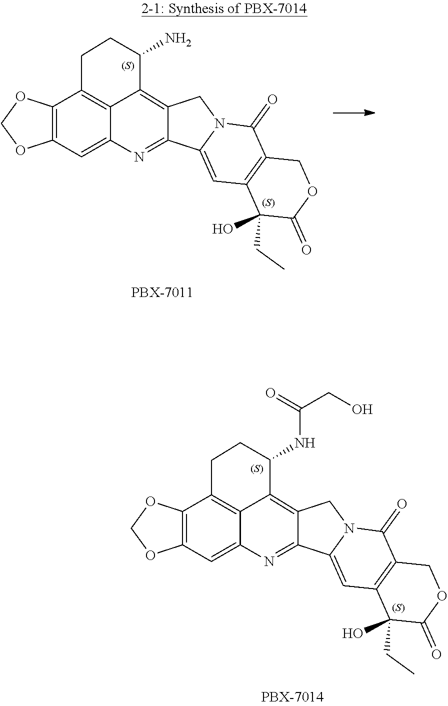

A stock solution of activated glycolic acid was prepared according to the following procedure:

Glycolic acid (17 mg, 0.224 mmol) was dissolved in 1 mL of N,N-dimethylformamide. HOSu (25.7 mg, 0.223 mmol) and EDC (42.8 mg, 0.223 mmol) were added. The reaction mixture was stirred at room temperature for 1 hour.

Subsequently, 0.4 mL of activated acid solution was added to a suspension of (1S,10S)-1-amino-10-ethyl-10-hydroxy-1,2,3,10,13,16-hexahydro-11H,14H-benzo[de][1,3]dioxolo[4,5-g]pyrano[3′,4′:6,7]indolizino[1,2-b]quinolin-11,14-dione (40 mg, 0.089 mmol) and triethylamine (0.025 mL, 0.179 mmol) in N,N-dimethylformamide (2.5 mL). The mixture was stirred at room temperature for 3 hours. 0.05 mL of freshly prepared active acid solution was added. Subsequently, the reaction mixture was further stirred at room temperature for 2 hours. The reaction mixture was dried by evaporation. A crude product was purified by column chromatography (0 to 8% methanol in chloroform). Thereby, a yellow solid containing the PBX-7014 product and residual succinimide was obtained. The product was further purified by acidic preparative MPLC (Luna5-40), and a product fraction was lyophilized, thereby obtaining a light yellow solid. Yield: 20 mg, 50%.

U_AN_ACID: m/z 506.2 [M+H]⁺

¹H NMR (400 MHz, DMSO-d6) δ 8.40 (d, J=8.9 Hz, 1H), 7.40 (s, 1H), 7.23 (s, 1H), 6.49 (s, 1H), 6.28 (d, J=4.6 Hz, 2H), 5.61-5.45 (m, 2H), 5.45-5.35 (m, 2H), 5.19-5.06 (m, 2H), 3.95 (s, 2H), 3.13-2.96 (m, 2H), 2.21-2.02 (m, 2H), 1.94-1.77 (m, 2H), 0.87 (t, J=7.3 Hz, 3H).

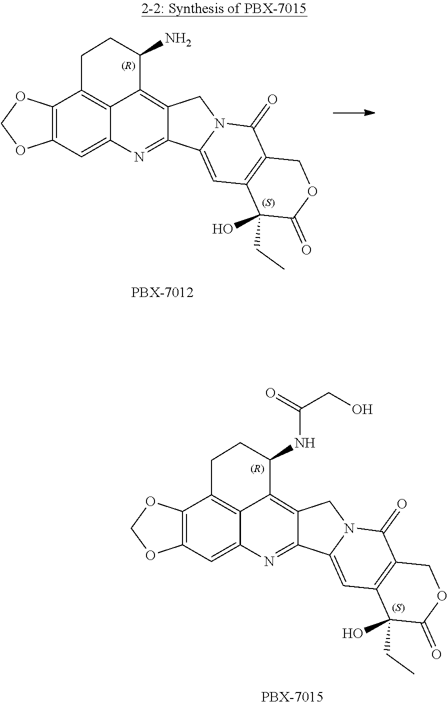

A stock solution of activated acid was prepared according to the following procedure:

Glycolic acid (17.00 mg, 0.223 mmol) was dissolved in 1 mL of N,N-dimethylformamide. HOSu (25.7 mg, 0.223 mmol) and EDC (42.8 mg, 0.223 mmol) were added. The reaction mixture was stirred at room temperature for 1 hour.

Subsequently, 0.25 mL of activated acid solution was added to a suspension of (1R,10S)-1-amino-10-ethyl-10-hydroxy-1,2,3,10,13,16-hexahydro-11H,14H-benzo[de][1,3]dioxolo[4,5-g]pyrano[3′,4′:6,7]indolizino[1,2-b]quinolin-11,14-dione (25 mg, 0.056 mmol) and triethylamine (0.016 mL, 0.112 mmol) in N,N-dimethylformamide (2.5 mL). The mixture was stirred overnight at room temperature. 0.03 mL of a freshly prepared activated acid solution was added. Subsequently, the reaction mixture was stirred at room temperature for 2 hours. The reaction mixture was combined with a previous smaller batch, and dried by evaporation. A crude product was purified by column chromatography. Thereby, a yellow solid containing the PBX-7015 product and residual succinimide was obtained. The product was purified by acidic preparative MPLC (Luna5-40). A product fraction was lyophilized to obtain a light yellow solid. Yield: 15 mg, 38%.

U_AN_ACID: 506.2 [M+H]⁺

¹H NMR (400 MHz, DMSO) δ 8.44 (d, J=9.0 Hz, 1H), 7.41 (s, 1H), 7.24 (s, 1H), 6.48 (s, 1H), 6.29 (d, J=2.3 Hz, 2H), 5.61-5.44 (m, 2H), 5.44-5.36 (m, 2H), 5.20-5.07 (m, 2H), 3.96 (s, 2H), 3.10-2.96 (m, 2H), 2.20-2.06 (m, 2H), 1.95-1.79 (m, J=7.3 Hz, 2H), 0.87 (t, J=7.3 Hz, 3H).

### Preparation Example 3: Synthesis of PBX-7016

This example illustrates the synthesis of compound PBX-7016 starting from the exatecan-hybrid compound (PBX-7011).

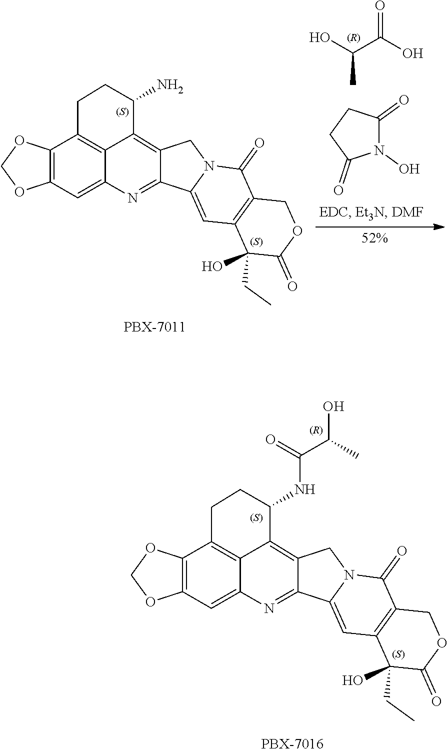

A stock solution of activated D-lactic acid was prepared as follows.

D-lactic acid (22 mg, 0.244 mmol) was dissolved in 1 mL of N,N-dimethylformamide. HOSu (27 mg, 0.235 mmol) and EDC (38.6 mg, 0.201 mmol) were added. The reaction mixture was stirred at room temperature for 2 hours.

Subsequently, 0.3 mL of activated acid solution was added to a solution of (1S,10S)-1-amino-10-ethyl-10-hydroxy-1,2,3,10,13,16-hexahydro-11H,14H-benzo[de][1,3]dioxolo[4,5-g]pyrano[3′,4′:6,7]indolizino[1,2-b]quinolin-11,14-dione (36 mg, 0.080 mmol) and triethylamine (0.022 mL, 0.161 mmol) in N,N-dimethylformamide (2.5 mL). The mixture was stirred at room temperature for 6 hours. 0.05 mL of the prepared active acid solution was added. Subsequently, the reaction mixture was stirred overnight at room temperature. The reaction mixture was directly purified by acidic preparative MPLC (Luna10-50), and a product fraction was lyophilized, thereby obtaining a light yellow solid.

Yield: 22 mg, 52%

U_AN_ACID: m/z 520.2 [M+H]⁺

¹H NMR (400 MHz, DMSO) δ 8.43 (d, J=9.1 Hz, 1H), 7.41 (s, 1H), 7.23 (s, 1H), 6.50 (s, 1H), 6.29 (d, J=2.1 Hz, 2H), 5.62-5.58 (m, 1H), 5.58-5.51 (m, 1H), 5.41 (s, 2H), 5.21-5.01 (m, 2H), 4.17-4.07 (m, 1H), 3.14-2.95 (m, 2H), 2.19-2.04 (m, 2H), 1.92-1.78 (m, 2H), 1.39 (d, J=6.8 Hz, 3H), 0.87 (t, J=7.3 Hz, 3H).

As described above, since PBX-7016 can be synthesized from PBX-7011, PBX-7017 can be synthesized from PBX-7012 in the same manner as described above.

### Preparation Example 4: Synthesis of PBX-7024

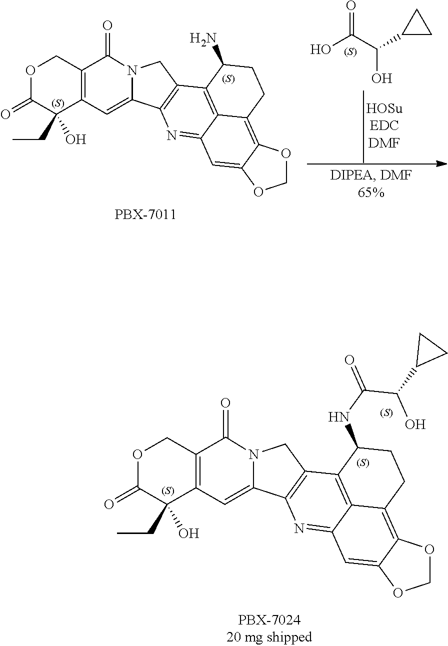

PBX-7024 was prepared with high yield by binding (2S)-2-cyclopropyl-2-hydroxyacetic acid to the PBX-7011 compound.

(2S)-2-cyclopropyl-2-hydroxyacetic acid (26 mg, 0.244 mmol) was dissolved in 1 mL of N,N-dimethylformamide. HOSu (26 mg, 0.226 mmol) and EDC (42 mg, 0.219 mmol) were added. The reaction mixture was stirred at room temperature for 2 hours. Subsequently, 0.6 mL of activated acid solution was added to a solution of (1S,10S)-1-amino-10-ethyl-10-hydroxy-1,2,3,10,13,16-hexahydro-11H,14H-benzo[de][1,3]dioxolo[4,5-g]pyrano[3′,4′:6,7]indolizino[1,2-b]quinolin-11,14-dione (40 mg, 0.089 mmol) and DIPEA (0.047 mL, 0.268 mmol) in N,N-dimethylformamide (2.5 mL). The mixture was stirred overnight at room temperature. The reaction mixture was directly purified by acidic preparative MPLC (Luna10-50), and a product fraction was lyophilized, thereby obtaining a light white solid.

Yield: 32 mg, 65%

U_AN_ACID: m/z 520.2 [M+H]⁺

¹H NMR (400 MHz, DMSO-d6) δ 8.33 (d, J=8.7 Hz, 1H), 7.39 (s, 1H), 7.23 (s, 1H), 6.48 (s, 1H), 6.28 (d, J=4.9 Hz, 2H), 5.50 (q, J=6.7 Hz, 1H), 5.40 (s, 3H), 5.23-5.06 (m, 2H), 3.62 (d, J=6.3 Hz, 1H), 3.03 (q, J=6.2 Hz, 2H), 2.21-2.02 (m, 2H), 1.92-1.79 (m, 2H), 1.18-1.08 (m, 1H), 0.87 (t, J=7.3 Hz, 3H), 0.47-0.30 (m, 4H).

### Preparation Example 5: Synthesis of 25-4 and 25-6 from PBX-7014 and PBX-7016 and Preparation of their ADCs (DAR7 and 8) (trastuzumab-25-4 and trastuzumab-25-6)

Linker-payloads, 25-4 (Chemical Formula 4) and 25-6 (Chemical Formula 5), which include two compounds (PBX-7014 and PBX-7016) and an enzymatically cleavable linker system GGFG were synthesized.

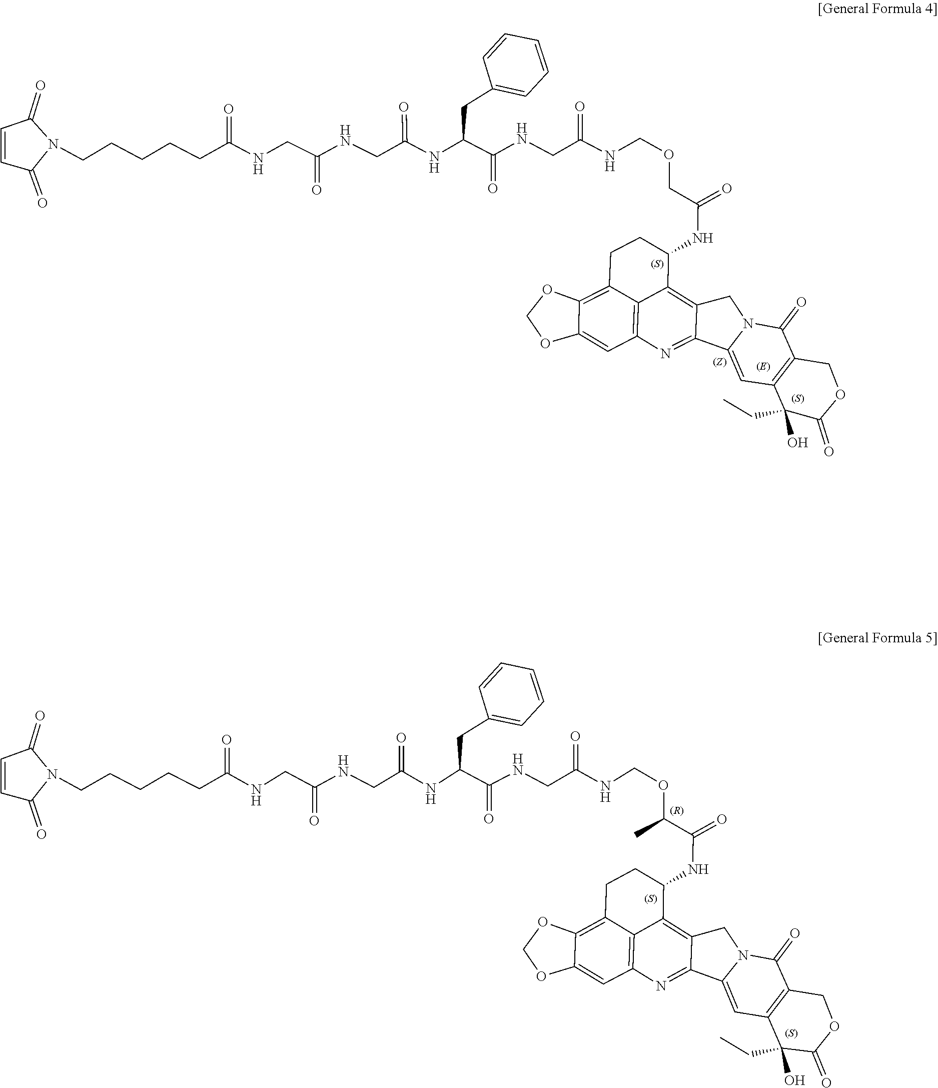

The molecular structure of 25-4 is GGFG-PBX-7014, and the molecular structure of 25-6 is GGFG-PBX-7016. That is, each uses the same GGFG linker as Enhertu®.

Further, ADCs were prepared with PBX-7014 and PBX-7016 as payloads using the same GGFG linker and trastuzumab antibody as Enhertu®.

The linker-payload compounds of Chemical Formula 4 and Chemical Formula 5 were conjugated with trastuzumab, which is a Her2 target antibody, thereby synthesizing ADCs (Tra-25-4 and Tra-25-6, both having DAR 8).

Trastuzumab-25-4 (trastuzumab-7014) was prepared as follows. After buffer exchange with a reaction buffer (150 mM NaCl, 50 mM histidine, pH 6.0) using a PD-10 desalting column, 27.5 μM of the prepared trastuzumab antibodies were treated with 825 μM TCEP at 25° C. for 2 hours to create a thiol site required for reaction from a disulfide of the antibody.

Afterward, excess TCEP was removed using a PD-10 desalting column, and a first conjugation reaction was carried out by allowing a 61.9 μM 25-4 drug linker (Chemical Formula 4) and 13.8 μM reduced trastuzumab to react in 10% DMSO-containing reaction buffer at 25° C. for 1 hour.

Trastuzumab-25-6 (trastuzumab-7016) was prepared according to the same process as above, and the same concentration of a 25-6 drug linker (Chemical Formula 5) was used instead of the 61.9 μM 25-4 drug linker (Chemical Formula 4).

Subsequently, each ADC was purified using SEC, confirming that it was purified with a monomer without aggregation. It was confirmed through SDS-PAGE that the drug was conjugated through the band shift of a light chain and a heavy chain.

### Preparation Example 6. Synthesis of CL2A-exatecan

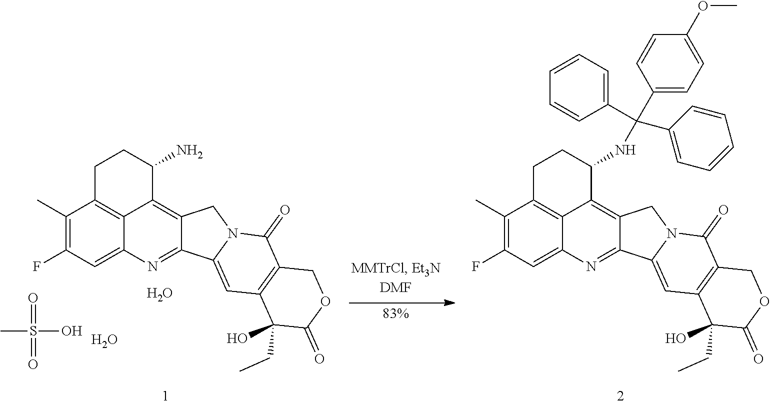

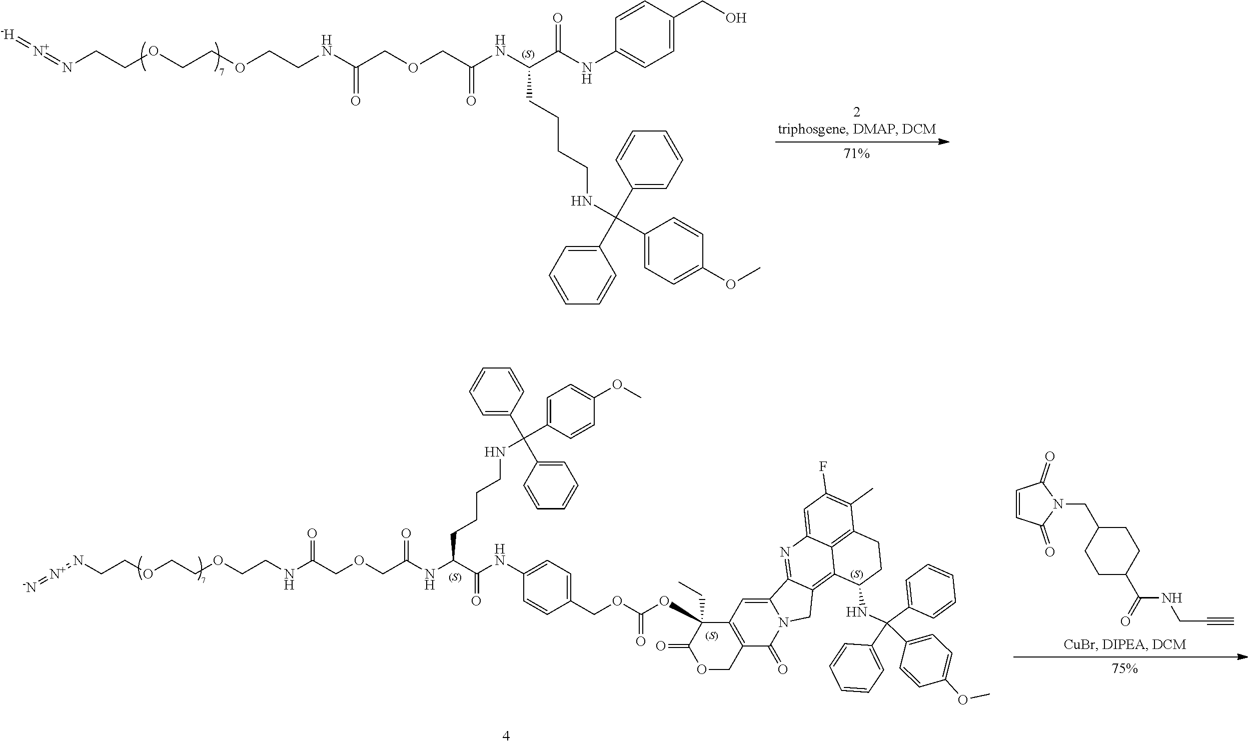

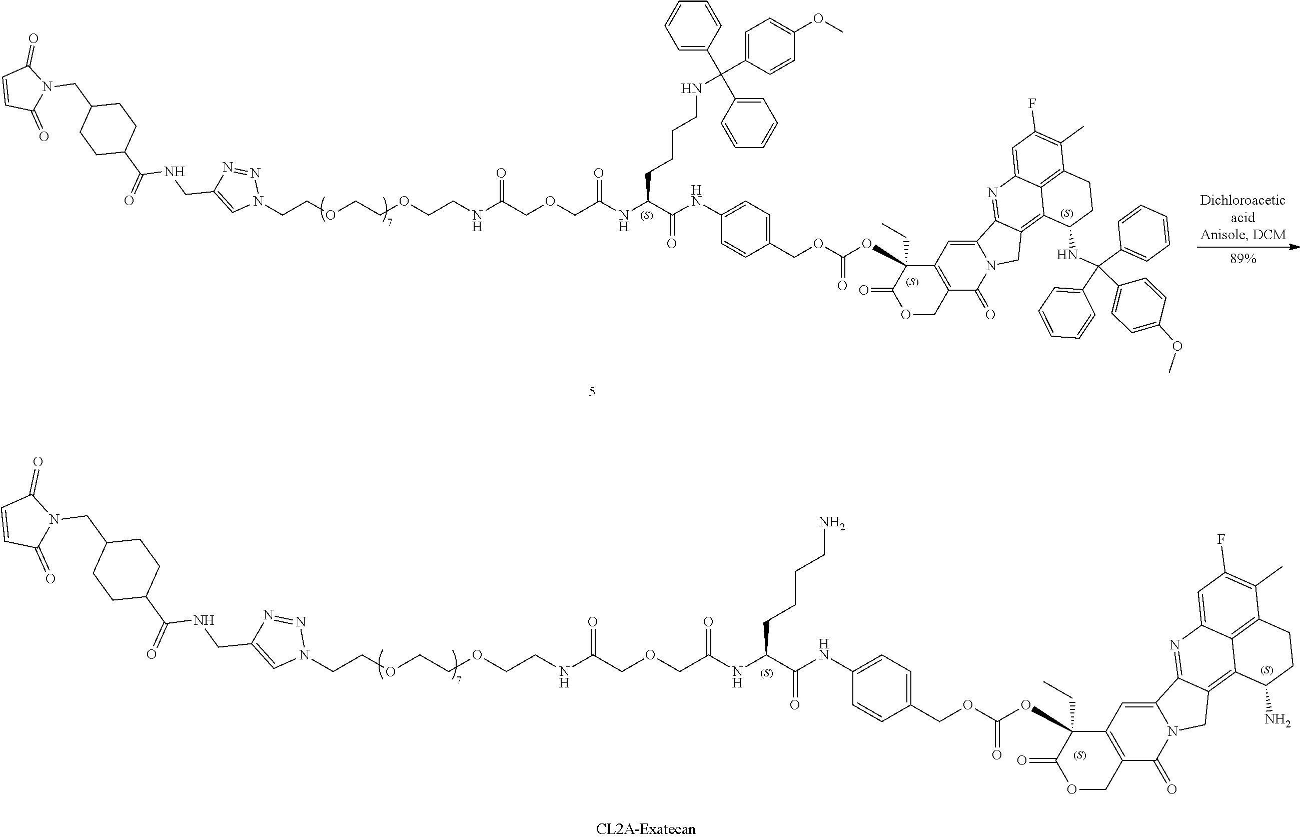

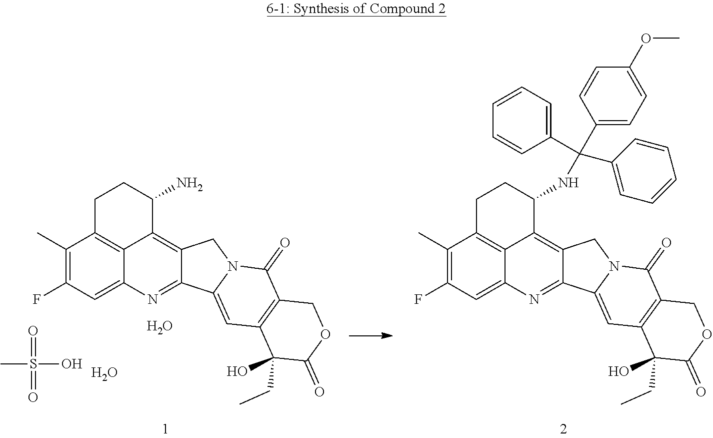

Triethylamine (0.098 mL, 0.705 mmol) was added to a suspension of exatecan mesylate dihydrate (100 mg, 0.176 mmol) in N,N-dimethylformamide (dry) (3 mL) under an argon atmosphere at room temperature. Subsequently, MMTrCl (109 mg, 0.352 mmol) was added. The reaction mixture was diluted with DMSO, purified through basic preparative MPLC (XSelect40-80), and freeze-dried, thereby obtaining (1S,9S)-9-ethyl-5-fluoro-9-hydroxy-1-(((4-methoxyphenyl)diphenylmethyl)amino)-4-methyl-1,2,3,9,12,15-hexahydro-10H,13H-benzo[de]pyrano[3′,4′:6,7]indolizino[1,2-b]quinoline-10,13-dione (103 mg, 83%) as an off-white solid.

SC_BASE: m/z 708.4 [M+H]⁺

¹H NMR (400 MHz, DMSO-d6) δ 7.73 (d, J=10.9 Hz, 1H), 7.58-7.49 (m, 4H), 7.45-7.38 (m, 2H), 7.37-7.27 (m, 5H), 7.27-7.20 (m, 2H), 6.88 (d, J=8.8 Hz, 2H), 6.49 (s, 1H), 5.42 (s, 2H), 5.17 (d, J=19.2 Hz, 1H), 4.91 (d, J=19.2 Hz, 1H), 4.00-3.90 (m, 1H), 3.74 (s, 3H), 3.63 (d, J=7.4 Hz, 1H), 3.23-3.11 (m, 1H), 2.80-2.68 (m, 1H), 2.33 (s, 3H), 1.96-1.80 (m, 2H), 1.67-1.55 (m, 1H), 1.31-1.19 (m, 1H), 0.88 (t, J=7.3 Hz, 3H).

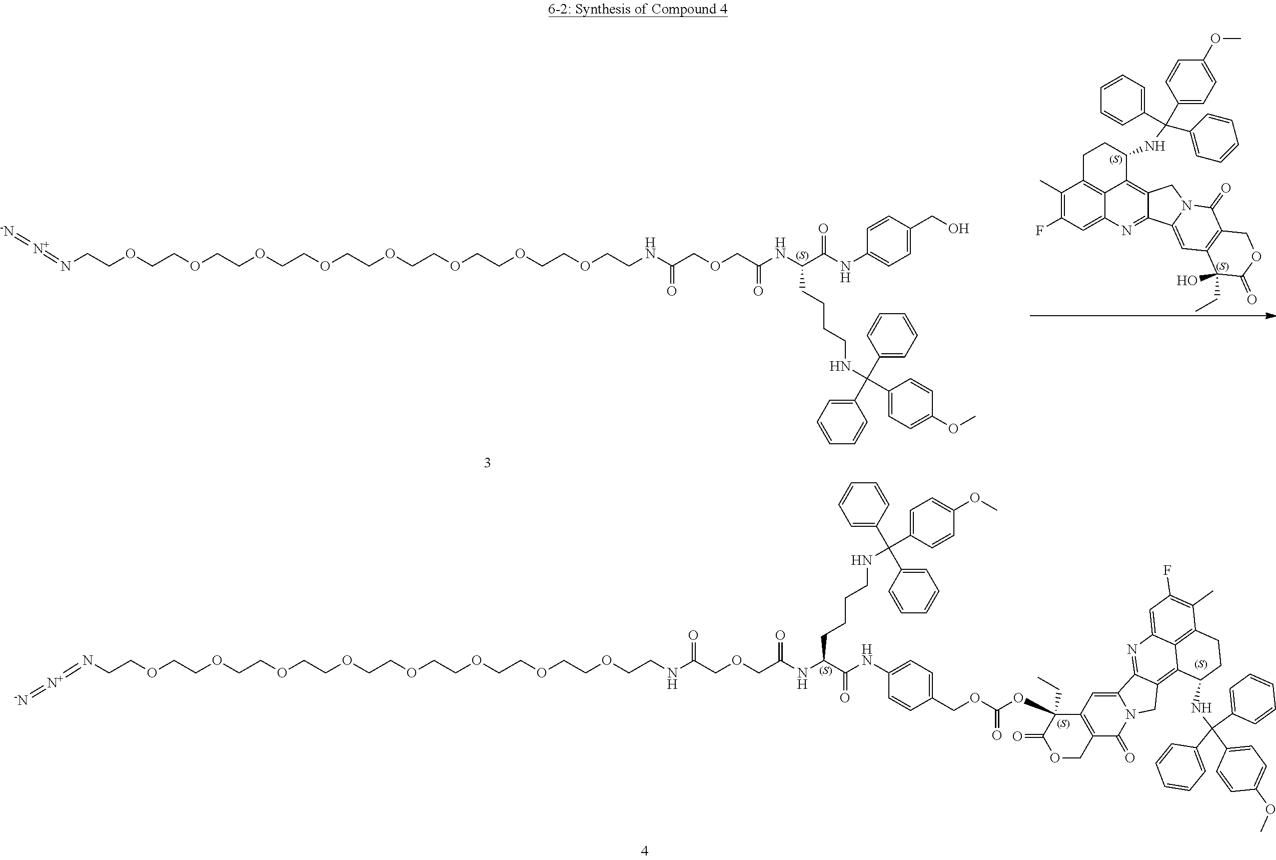

(1S,9S)-9-ethyl-5-fluoro-9-hydroxy-1-(((4-methoxyphenyl)diphenylmethyl)amino)-4-methyl-1,2,3,9,12,15-hexahydro-10H,13H-benzo[de]pyrano[3′,4′:6,7]indolizino[1,2-b]quinoline-10,13-dione (85 mg, 0.120 mmol) and DMAP (55 mg, 0.456 mmol) were added to a flask dried under a nitrogen atmosphere, and dissolved in dichloromethane (6 mL). After stirring for 5 minutes, a solution of triphosgene (14.2 mg, 0.048 mmol) in dichloromethane (0.750 m) was added at one time, and the reaction mixture was stirred at room temperature for 15 minutes. After 10 minutes, LCMS analysis (sample in MeOH) showed that the reaction mixture was well converted into methyl carbonate. An (S)-2-(32-azido-5-oxo-3,9,12,15,18,21,24,27,30-nonaoxa-6-azadotriacontanamido)-N-(4-(hydroxymethyl)phenyl)-6-(((4-methoxyphenyl)diphenylmethyl)amino)hexaneamide solution (140 mg, 0.132 mmol) was added to dichloromethane (1.5 mL), and the reaction mixture was stirred at room temperature for 30 minutes. The reaction mixture was concentrated under reduced pressure, dissolved in DMSO, purified through basic preparative MPLC (XSelect50-100), and freeze-dried, thereby obtaining a product (153 mg, 71%) as an off-white solid.

SC_BASE_M1800: m/z 1793.5 [M+H]⁺

¹H NMR (400 MHz, CDCl₃) δ 8.60 (s, 1H), 7.69 (d, J=10.6 Hz, 1H), 7.59-7.49 (m, 6H), 7.44 (d, J=8.4 Hz, 6H), 7.38-7.28 (m, 8H), 7.24-7.09 (m, 6H), 6.89-6.74 (m, 4H), 5.65 (d, J=17.3 Hz, 1H), 5.36 (d, J=17.2 Hz, 1H), 5.11 (d, J=12.0 Hz, 1H), 4.97 (d, J=12.1 Hz, 1H), 4.69-4.44 (m, 3H), 4.21-3.97 (m, 5H), 3.84-3.74 (m, 6H), 3.68-3.51 (m, 33H), 3.50-3.41 (m, 2H), 3.40-3.27 (m, 3H), 2.96-2.85 (m, 1H), 2.43 (s, 3H), 2.33-1.79 (m, 7H), 1.78-1.34 (m, 14H), 0.97 (t, J=7.4 Hz, 3H).

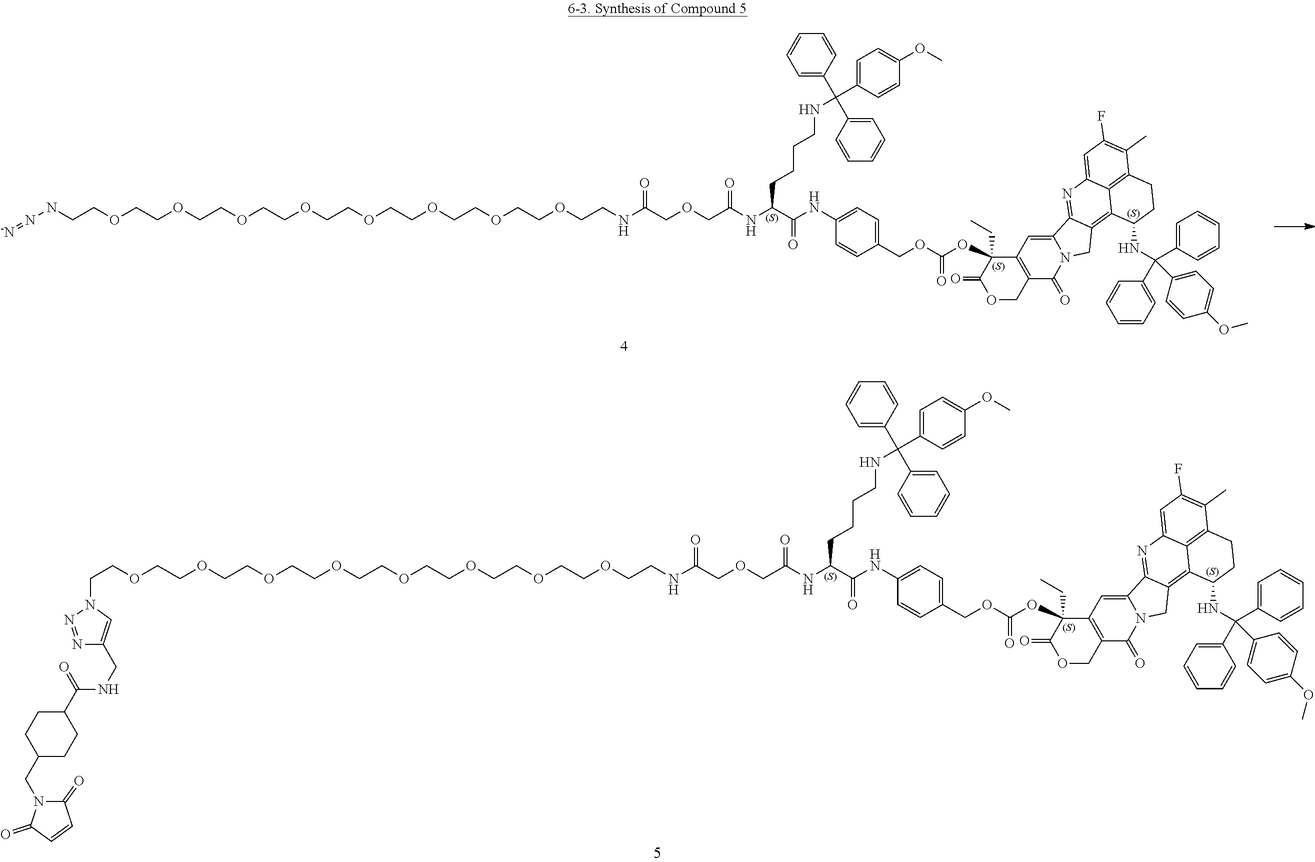

4-((2,5-dioxo-2,5-dihydro-1H-pyrrol-1-yl)methyl)-N-(prop-2-in-1-yl)cyclohexane-1-carboxamide (0.070 g, 0.256 mmol), copper (I) bromide (7.3 mg, 0.051 mmol), and DIPEA (0.045 mL, 0.256 mmol) were added to a 4-((S)-35-azido-2-(4-(((4-methoxyphenyl)diphenylmethyl)amino)butyl)-4, 8-dioxo-6,12,15,18,21,24,27,30,33-nonaoxa-3,9-diazapentatriacontanamido)benzyl ((1S,9S)-9-ethyl-5-fluoro-1-(((4-methoxyphenyl)diphenylmethyl)amino)-4-methyl-10,13-dioxo-2,3,9,10,13,15-hexahydro-1H,12H-benzo[de]pyrano[3′,4′:6,7]indolizino[1,2-b]quinolin-9-yl)carbonate solution (0.153 g, 0.085 mmol) in dichloromethane (9 mL). The reaction mixture was stirred overnight at room temperature. An additional amount of copper (I) bromide (7.3 mg, 0.051 mmol) was added. After stirring for 3 hours, a product was increased in Sample-2 on LCMS. The reaction mixture was further stirred for 5 hours and concentrated under reduced pressure. The residue was purified by basic preparative MPLC (XSelect50-100) for basic fractionation and freeze-dried, thereby obtaining a product (133 mg, 75%) as an off-white solid.

SC_BASE_M1800: m/z 1795.5 [M-MMT+H]⁺, 1523.5 [M-2×MMT+H]⁺

¹H NMR (400 MHz, DMSO-d6) δ 10.16 (s, 1H), 8.24-8.11 (m, 2H), 8.11-8.02 (m, 1H), 7.81 (s, 1H), 7.78-7.69 (m, 1H), 7.63-7.49 (m, 6H), 7.44-7.08 (m, 27H), 7.03-6.96 (m, 3H), 6.89 (d, J=8.5 Hz, 2H), 6.85-6.78 (m, 2H), 5.60-5.45 (m, 2H), 5.30-4.97 (m, 4H), 4.51-4.39 (m, 3H), 4.24 (d, J=5.6 Hz, 2H), 4.08-3.93 (m, 5H), 3.83-3.59 (m, 10H), 3.53-3.36 (m, 36H), 3.27-3.19 (m, 6H), 2.22-1.86 (m, 7H), 1.81-1.38 (m, 13H), 1.38-1.11 (m, 7H), 0.90 (t, J=7.3 Hz, 4H), 0.87-0.80 (m, 1H).

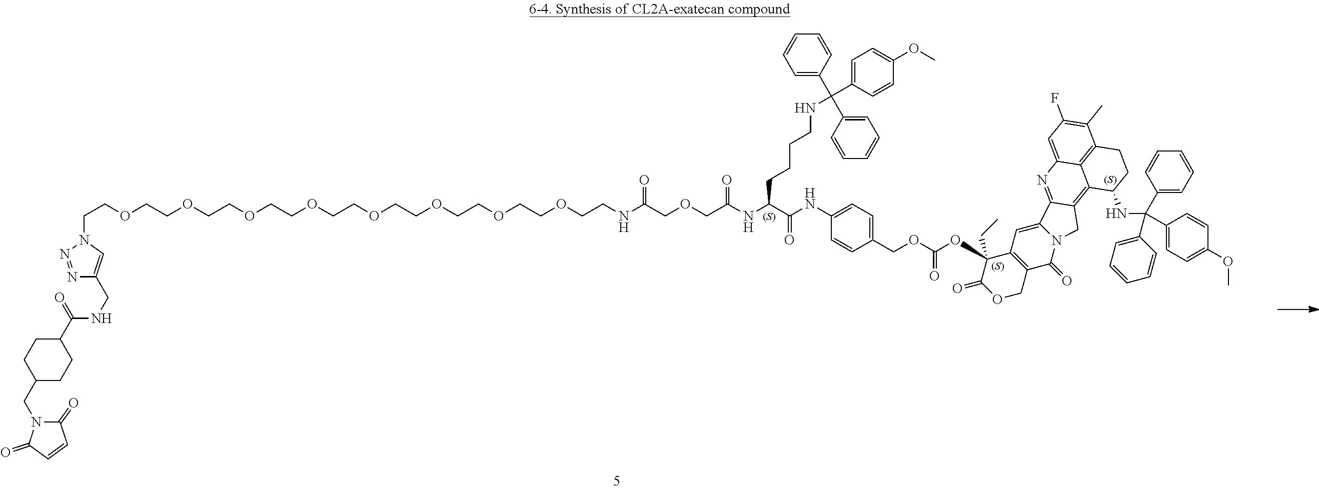

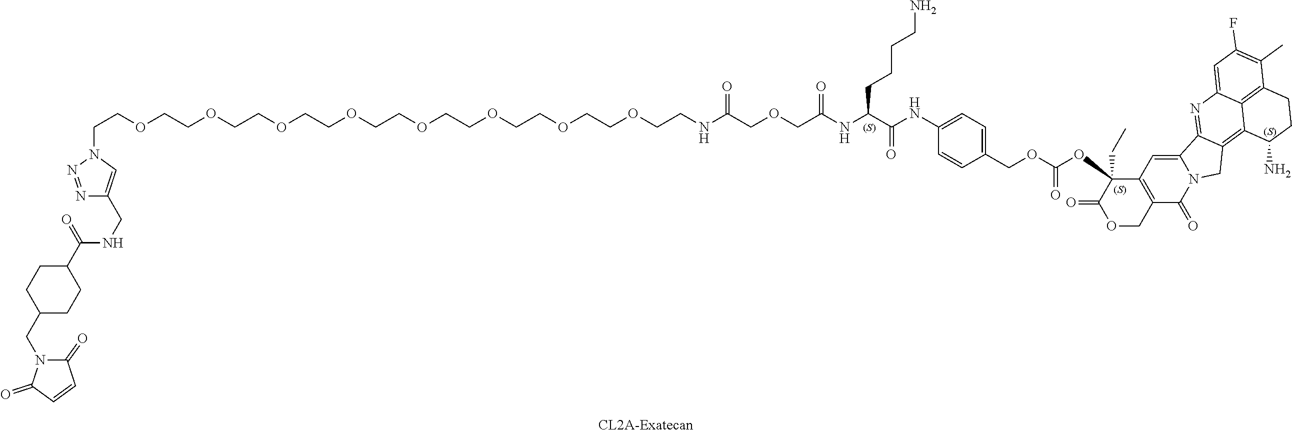

Anisole (0.528 mL, 4.83 mmol) was added to a 4-((S)-35-(4-((4-((2,5-dioxo-2,5-dihydro-1H-pyrrol-1-yl)methyl)cyclohexane-1-carboxamido)methyl)-1H-1,2,3-triazole-1-yl)-2-(4-(((4-methoxyphenyl)diphenylmethyl)amino)butyl)-4,8-dioxo-6,12,15,18,21,24,27,30,33-nonaoxa-3,9-diazapentatriacontanamido)benzyl ((1S,9S)-9-ethyl-5-fluoro-1-(((4-methoxyphenyl)diphenylmethyl)amino)-4-methyl-10,13-dioxo-2,3,9,10,13,15-hexahydro-1H,12H-benzo[de]pyrano[3′,4′:6,7]indolizino[1,2-b]quinolin-9-yl)carbonate solution (100 mg, 0.048 mmol) in dichloromethane (anhydrous) (1.5 mL) at room temperature under an inert atmosphere, and dichloroacetic acid (0.160 mL, 1.934 mmol) was added dropwise. Subsequently, MTBE (up to 3 m) was added. The reaction mixture was changed into a fine suspension. Heptane (to 3 mL) was added. As much of the solvent as possible was removed using a pipette. The residue was washed with an MTBE/heptane (1:1, up to 4 mL) mixture several times. The wet residue was died under reduced pressure overnight, thereby obtaining a pale green solid (85 mg, 89%).

AN_ACID: m/z 763.0 [M+2H]²⁺/2

¹H NMR (400 MHz, DMSO-d6) δ 10.18 (s, 1H), 8.26-8.14 (m, 2H), 8.08 (t, J=5.7 Hz, 1H), 7.89 (d, J=10.8 Hz, 1H), 7.81 (s, 1H), 7.78-7.50 (m, 5H), 7.31 (d, J=8.3 Hz, 2H), 7.07 (s, 1H), 7.01 (s, 2H), 6.17 (s, 3H), 5.79-5.41 (m, 4H), 5.19-4.99 (m, 3H), 4.52-4.42 (m, 3H), 4.25 (d, J=5.6 Hz, 2H), 4.09-3.94 (m, 4H), 3.78 (t, J=5.3 Hz, 2H), 3.55-3.41 (m, 33H), 3.25-3.19 (m, 4H), 2.83-2.72 (m, 2H), 2.44-2.42 (m, 3H), 2.26-2.00 (m, 5H), 1.80-1.46 (m, 10H), 1.44-1.18 (m, 6H), 0.95-0.85 (m, 5H).

### Preparation Example 7: Synthesis of CL2A-Dxd

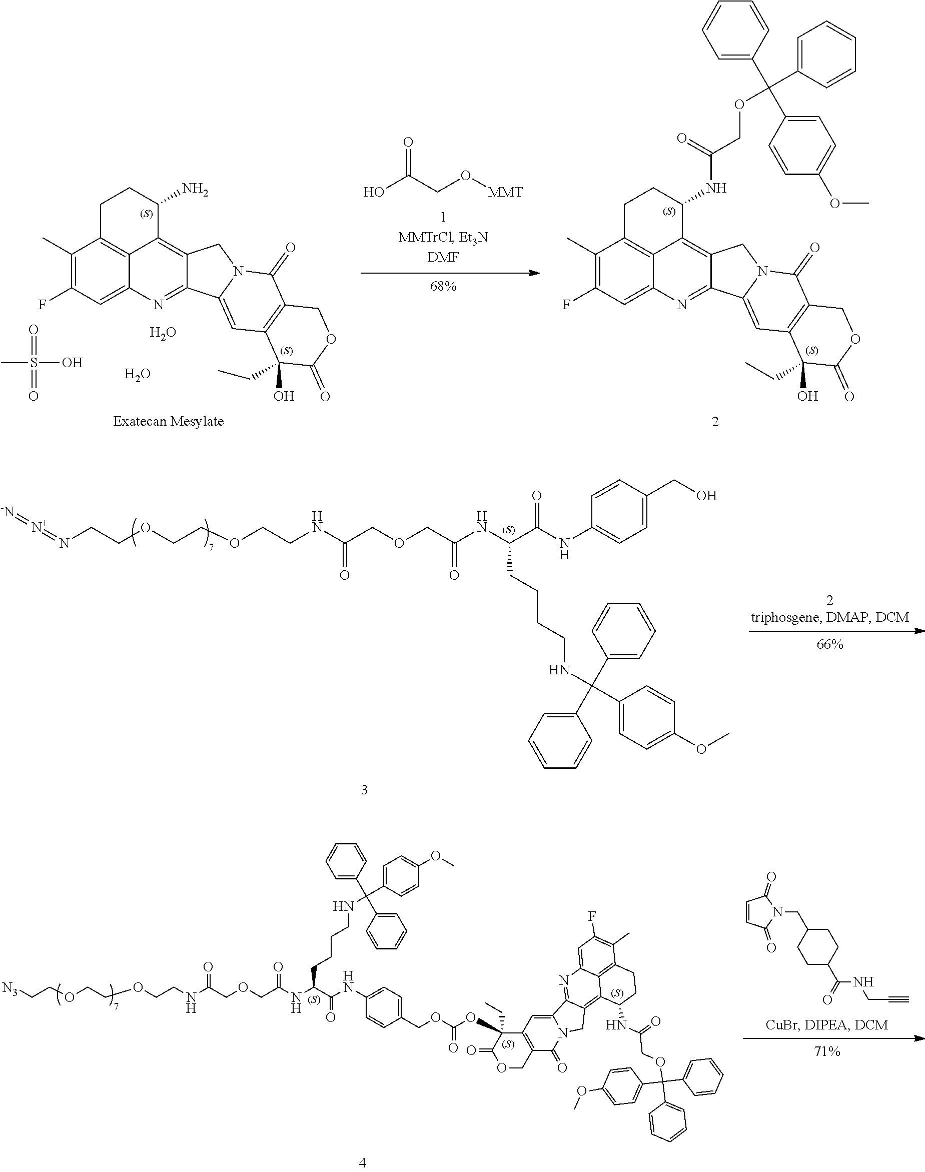

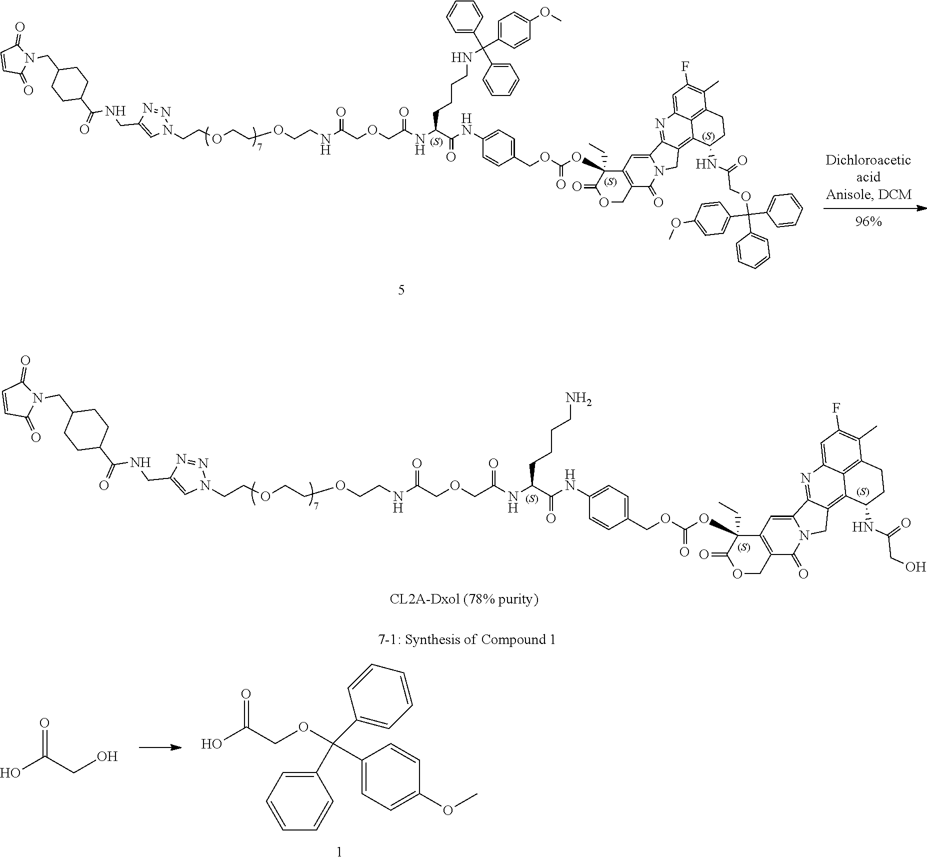

2-hydroxyacetic acid (133 mg, 1.749 mmol) and triethylamine (0.729 mL, 5.25 mmol) were dissolved in dichloromethane (4 mL). The reaction mixture was cooled to 0° C. Subsequently, an MMTrCl (702 mg, 2.273 mmol) solution in dichloromethane (4.00 mL) was added under an argon atmosphere. The reaction mixture was slowly allowed to reach room temperature and stirred for 2 days. The reaction mixture was concentrated under reduced pressure. The residue was purified by flash chromatography (silica, 0% to 10% methanol+1% TEA in dichloromethane), thereby obtaining a white solid contaminated with residual triethylamine as a product. Yield: 500 mg, 30% (corrected for residual triethylamine). The product was used without further purification.

SC_BASE: m/z 347.2 [M+H]⁺

¹H NMR (400 MHz, CDCl₃) δ 7.59-7.50 (m, 4H), 7.45-7.38 (m, 2H), 7.28-7.13 (m, 6H), 6.83-6.76 (m, 2H), 3.77 (s, 3H), 3.59 (s, 2H).

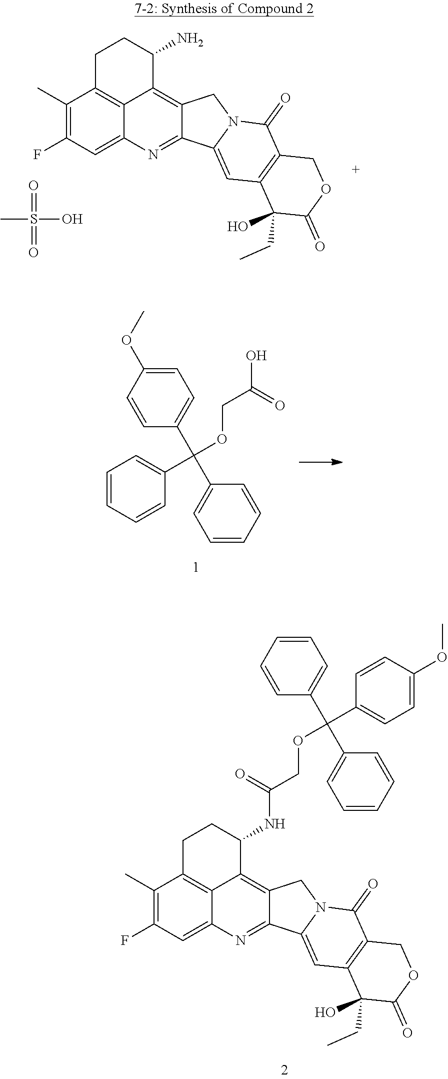

NHS (29.2 mg, 0.254 mmol) and EDCI·HCl (48.7 mg, 0.254 mmol) were added to a solution of 2-((4-methoxyphenyl)diphenylmethoxy)acetic acid (126 mg, 0.254 mmol) in N,N-dimethylformamide (dry) (1.2 mL). The reaction mixture was stirred at room temperature for 1 hour. Subsequently, this solution was added to a suspension of exatecan mesylate (90 mg, 0.169 mmol) and triethylamine (0.026 mL, 0.186 mmol) in N,N-dimethylformamide(dry) (1.2 mL) and stirred overnight at room temperature. The reaction mixture was diluted with DMSO and purified by basic preparative MPLC (XSelect30-70), concentrated from a mixture of acetonitrile and water (1:1, 10 mL) and freeze-dried, thereby obtaining N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinoline-1-yl)-2-((4-methoxyphenyl)diphenylmethoxy)acetamide as an off-white solid. Yield: 89 mg, 68%.

SC_BASE: m/z 766.4 [M+H]⁺

¹H NMR (400 MHz, CDCl₃) δ 7.74 (d, J=10.6 Hz, 1H), 7.61 (s, 1H), 7.26-7.09 (m, 13H), 6.90 (d, J=9.1 Hz, 1H), 6.75-6.69 (m, 2H), 5.76 (d, J=16.4 Hz, 1H), 5.61-5.52 (m, 1H), 5.35-5.20 (m, 3H), 4.03-3.93 (m, 2H), 3.74 (s, 3H), 3.22-3.12 (m, 1H), 3.10-2.99 (m, 1H), 2.44 (s, 3H), 2.27-2.19 (m, 2H), 1.98-1.84 (m, 2H), 1.06 (t, J=7.4 Hz, 3H).

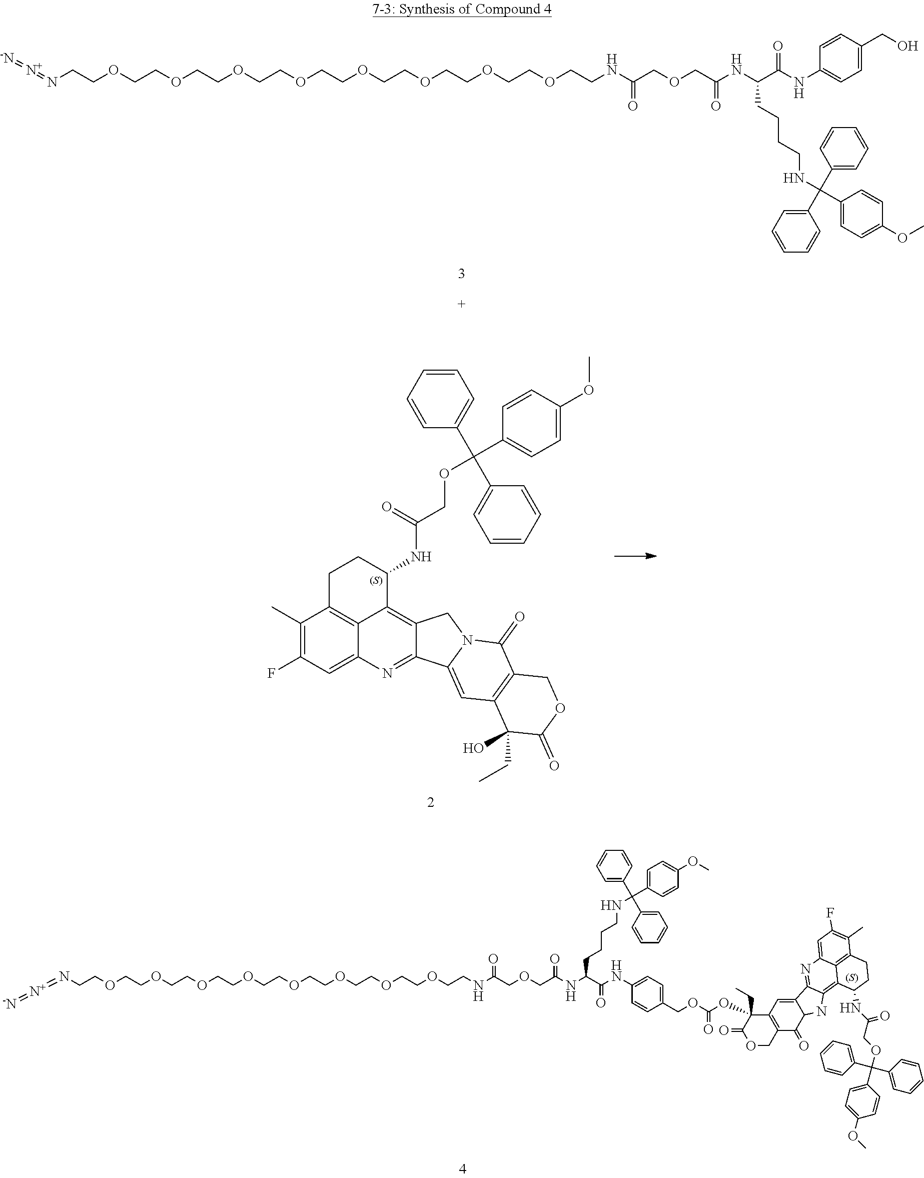

N-((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-((4-methoxyphenyl)diphenylmethoxy)acetamide (80 mg, 0.104 mmol) and DMAP (48.5 mg, 0.397 mmol) were dissolved in dichloromethane (6 mL) in a flask dried under a nitrogen atmosphere. After stirring for 5 minutes, a solution of triphosgene (12.4 mg, 0.042 mmol) in dichloromethane (0.750 mL) was added at one time, and the reaction mixture was stirred at room temperature for 15 minutes. A solution of (S)-2-(32-azido-5-oxo-3,9,12,15,18,21,24,27,30-nonaoxa-6-azadotriacontanamido)-N-(4-(hydroxymethyl))phenyl)-6-(((4-methoxyphenyl)diphenylmethyl)amino)hexaneimide (122 mg, 0.115 mmol) in dichloromethane (1.5 mL) was added, and the reaction mixture was stirred at room temperature for 30 minutes. The reaction mixture was concentrated under reduced pressure, dissolved in DMSO, purified by basic preparative MPLC (XSelect50-100), and freeze-dried, thereby obtaining a colorless solid. Yield: 0.129 g, 66%.

SC_BASE_M1900: m/z 1580.0 [M-MMT+H]⁺

¹H NMR (400 MHz, DMSO-d6) δ 10.14 (s, 1H), 8.32 (d, J=8.7 Hz, 1H), 8.13 (d, J=8.0 Hz, 1H), 8.10-8.01 (m, 1H), 7.80 (d, J=10.9 Hz, 1H), 7.58 (d, J=8.5 Hz, 2H), 7.45-7.33 (m, 8H), 7.33-7.11 (m, 19H), 7.02 (s, 1H), 6.89-6.78 (m, 4H), 5.56 (s, 1H), 5.50 (s, 2H), 5.33-5.15 (m, 2H), 5.09 (q, J=12.2 Hz, 2H), 4.47-4.40 (m, 1H), 4.00 (s, 2H), 3.96 (d, J=4.2 Hz, 2H), 3.71 (s, 3H), 3.70-3.68 (m, 3H), 3.64-3.57 (m, 4H), 3.56-3.52 (m, 4H), 3.52-3.47 (m, 24H), 3.43-3.36 (m, 5H), 3.28-3.21 (m, 3H), 3.13 (s, 2H), 2.42-2.36 (m, 4H), 2.24-2.10 (m, 4H), 2.00-1.88 (m, 2H), 1.73-1.53 (m, 2H), 1.53-1.42 (m, 2H), 1.41-1.22 (m, 3H), 0.90 (t, J=7.4 Hz, 3H).

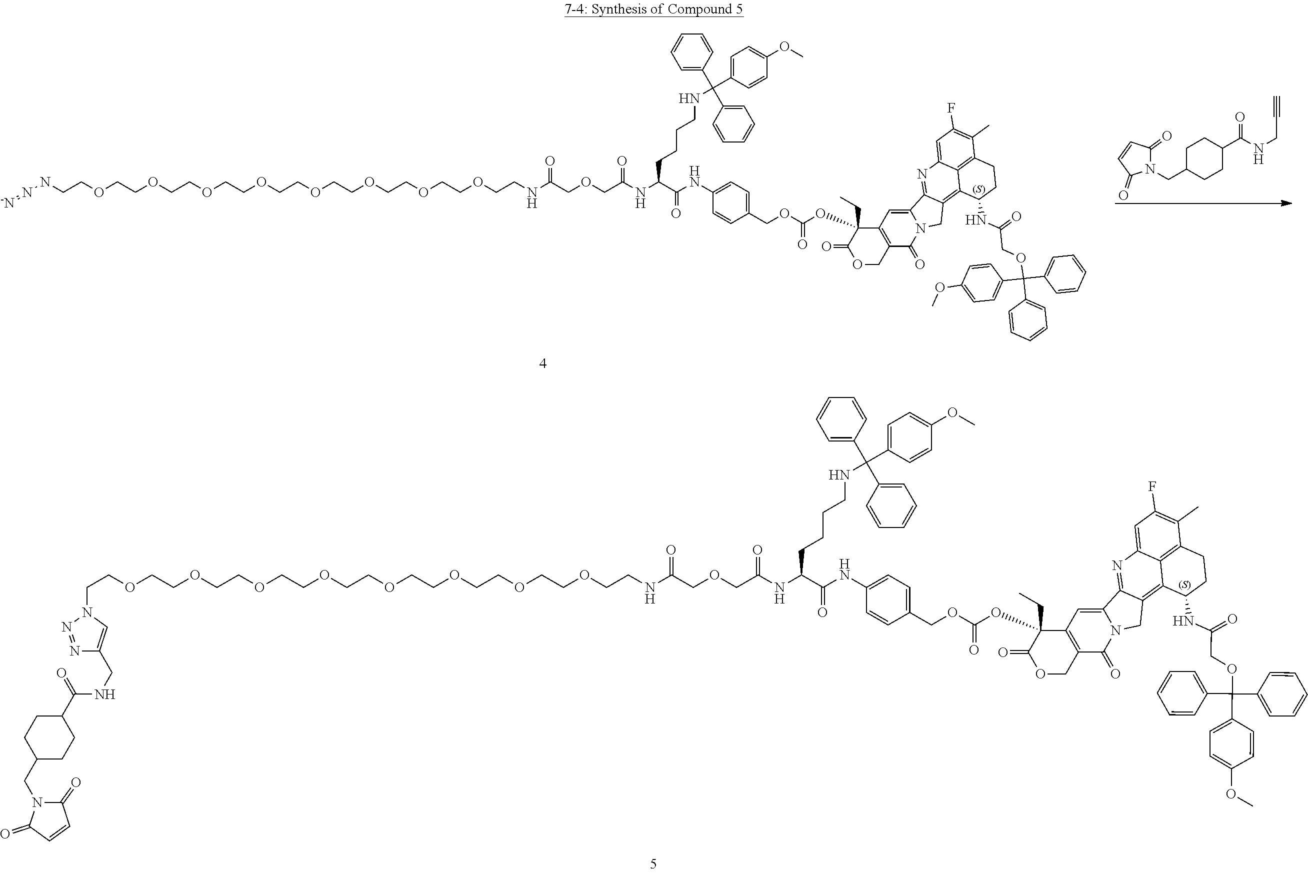

4-((2,5-dioxo-2,5-dihydro-1H-pyrrole-1-yl)methyl)-N-(prop-2-in-1-yl)cyclohexane-1-carbozmide (57 mg, 0.209 mmol), copper (I) bromide (6 mg, 0.042 mmol) and DIPEA (0.036 mL, 0.209 mmol) were added to a solution of 4-((S)-35-azido-2-(4-(((4-methoxyphenyl)diphenylmethyl)amino)butyl)-4, 8-dioxo-6,12,15,18,21,24,27,30,33-nonaoxa-3,9-diazapentatriacontanamido)benzyl ((1S,9R)-9-ethyl-5-fluoro-1-(2-((4-methoxyphenyl)diphenylmethoxy)acetamido)-4-methyl-10,13-dioxo-2,3,9,10,13,15-hexahydro-1H,12H-benzo[de]pyrano[3′,4′:6,7]indolizino[1,2-b]quinolin-9-yl)carbonate (0.129 g, 0.070 mmol) in dichloromethane (7 mL). The reaction mixture was stirred overnight at room temperature. An additional amount of copper (I) bromide (6 mg, 0.042 mmol) was added. After further stirring for 3 hours, the reaction mixture was concentrated under reduced pressure. The residue was purified by basic preparative MPLC (XSelect50-100). A product fraction was freeze-dried, thereby obtaining an off-white solid. Yield: 106 mg, 71%.

SC_BASE: m/z 1854.2 [M-MMT+H]⁺, 1582.0 [M-2×MMT+H]⁺

1H NMR (400 MHz, DMSO-d6) δ 10.15 (s, 1H), 8.34 (d, J=8.6 Hz, 1H), 8.24-8.03 (m, 3H), 7.87-7.73 (m, 2H), 7.67-7.53 (m, 2H), 7.44-7.34 (m, 9H), 7.30-7.10 (m, 19H), 7.07-6.99 (m, 2H), 6.90-6.77 (m, 5H), 5.61-5.39 (m, 2H), 5.29-4.99 (m, 4H), 4.53-4.38 (m, 4H), 4.32-4.20 (m, 2H), 4.02-3.93 (m, 5H), 3.78-3.66 (m, 10H), 3.62 (d, J=9.7 Hz, 2H), 3.52-3.38 (m, 38H), 3.25-3.20 (m, 3H), 2.42-2.36 (m, 3H), 2.24-2.10 (m, 3H), 1.99-1.87 (m, 3H), 1.76-1.55 (m, 6H), 1.55-1.41 (m, 4H), 1.29 (s, 6H), 0.90 (t, J=7.0 Hz, 4H).

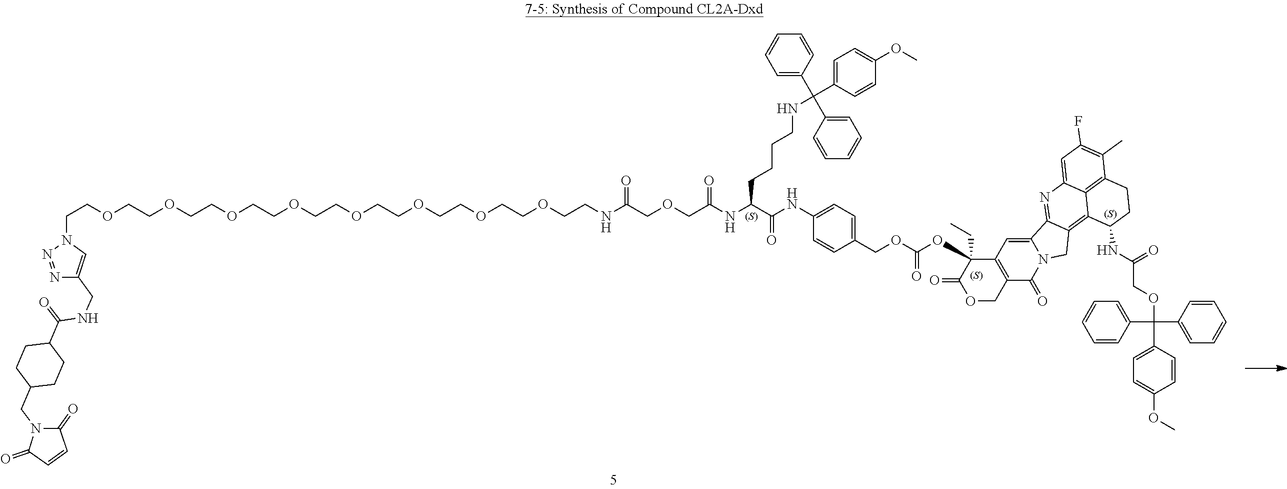

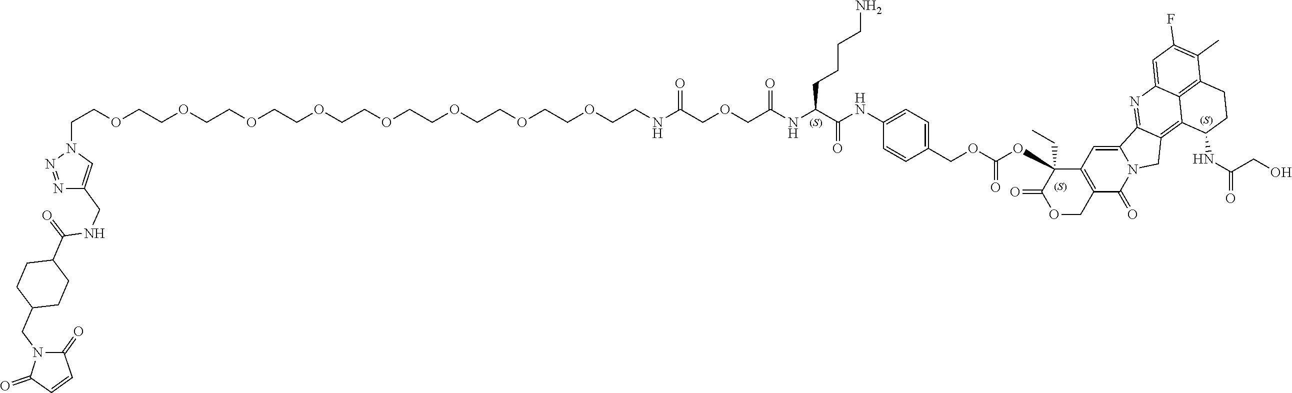

Anisole (0.411 mL, 3.76 mmol) was added to a solution of 4-((S)-35-(4-((4-((2,5-dioxo-2,5-dihydro-1H-pyrrole-1-yl)methyl)cyclohexane-1-carboxamido)methyl)-1H-1,2,3-triazole-1-yl)-2-(4-(((4-methoxyphenyl)diphenylmethyl)amino)butyl)-4,8-dioxo-6,12,15,18,21,24,27,30,33-nonaoxa-3,9-diazapentatriacontanamido)benzyl((1S,9R)-9-ethyl-5-fluoro-1-(2-((4-methoxyphenyl)diphenylmethoxy)acetamido)-4-methyl-10,13-dioxo-2,3,9,10,13,15-hexahydro-1H,12H-benzo[de]pyrano[3′,4′:6,7]indolizino[1,2-b]quinolin-9-yl)carbonate (80 mg, 0.038 mmol) in dichloromethane(anhydrous) (1.14 mL) at room temperature under an argon atmosphere, and then dichloroacetic acid (0.047 mL, 0.564 mmol) was added dropwise. After stirring for 1 hour, MTBE (up to 3 mL) was added. The reaction mixture changed into a fine suspension. Heptane (about 3 mL) was added to cause precipitation. As much of the solvent as possible was removed using a pipette. The residue was washed with a mixture of MTBE/heptane (1:1, about 4 mL) several times. The wet residue was dried under reduced pressure, transferred to a vial, and dried overnight under vacuum, thereby obtaining an off-white solid. Yield: 57 mg, 96%. Purity (LC-UV: 78%).

AN_ACID: 792.0 [M+2H]²⁺/2

¹H NMR analysis contains the expected signals.

¹H NMR (400 MHz, DMSO-d6) δ 10.18 (s, 1H), 8.43 (d, J=9.0 Hz, 1H), 8.26-8.15 (m, 2H), 8.12-8.03 (m, 1H), 7.86-7.53 (m, 8H), 7.45-7.18 (m, 6H), 7.05-6.97 (m, 3H), 6.19 (s, 2H), 5.66-5.54 (m, 1H), 5.50 (s, 3H), 5.23 (s, 2H), 5.17-5.00 (m, 2H), 4.52-4.44 (m, 3H), 4.25 (d, J=5.7 Hz, 2H), 4.06-3.92 (m, 7H), 3.81-3.74 (m, 3H), 3.74-3.68 (m, 1H), 3.58-3.46 (m, 33H), 3.23 (d, J=7.1 Hz, 7H), 2.40 (s, 3H), 2.27-1.98 (m, 7H), 1.82-1.44 (m, 11H), 1.28 (d, J=12.2 Hz, 6H), 0.95-0.81 (m, 6H).

### Preparation Example 8: CL2A/FL118-sacituzumab

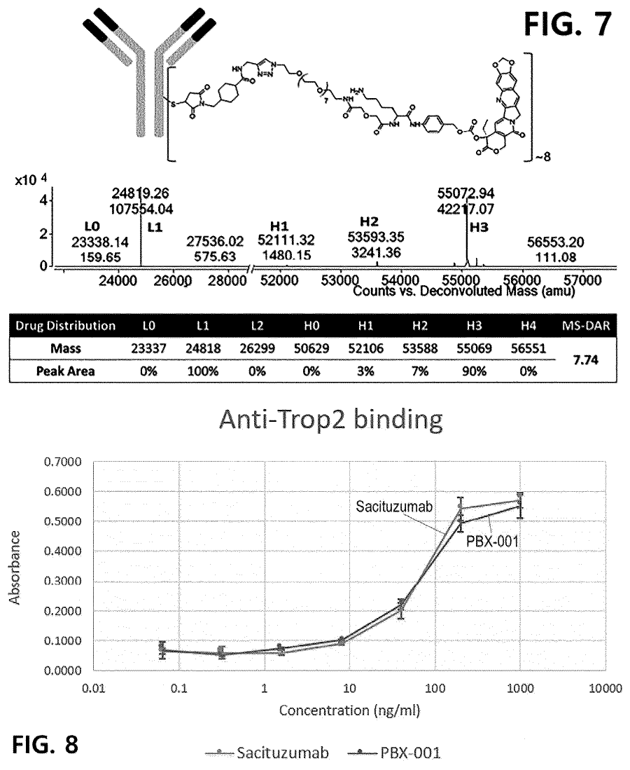

According to the method described in an Example of Korean Patent Publication No. 10-2349925, an ADC with DAR 8 was prepared using the combination of a monoclonal Ab with an IgG structure, i.e., sacituzumab, and a CL2A/FL118 linker-payload having maleimide at an end as a linker-payload. An ADC with a structure of DAR 8 was confirmed using HIC, SEC, and mass spectrometry (FIG. 7).

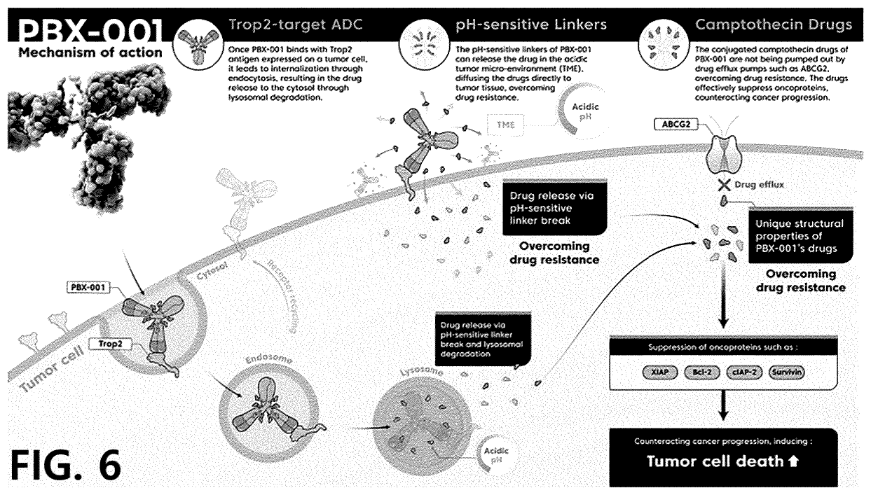

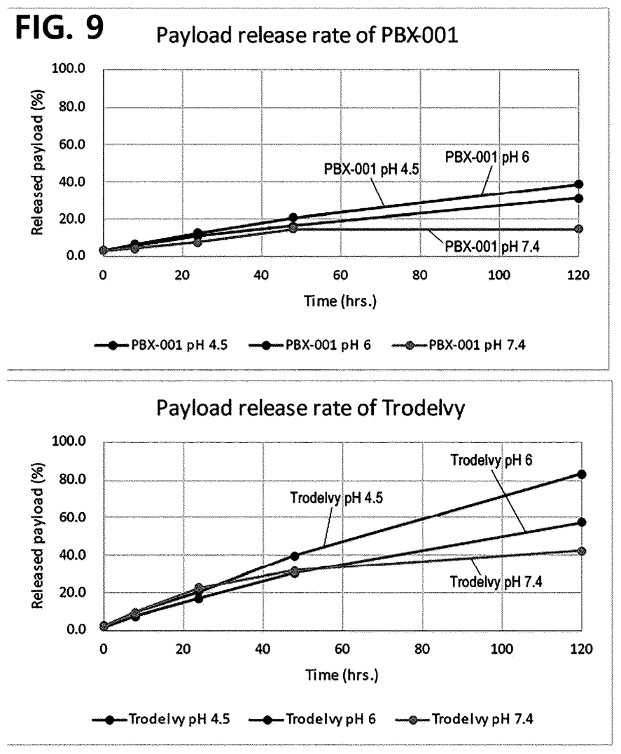

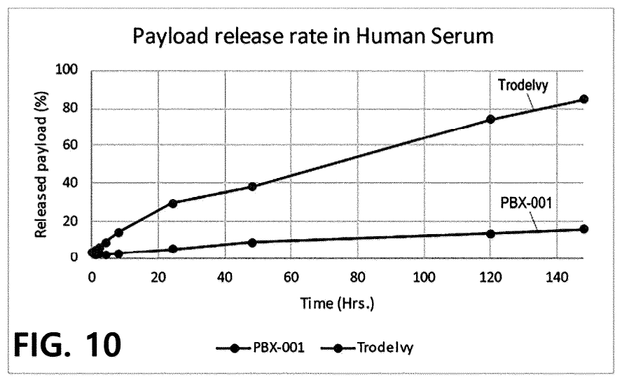

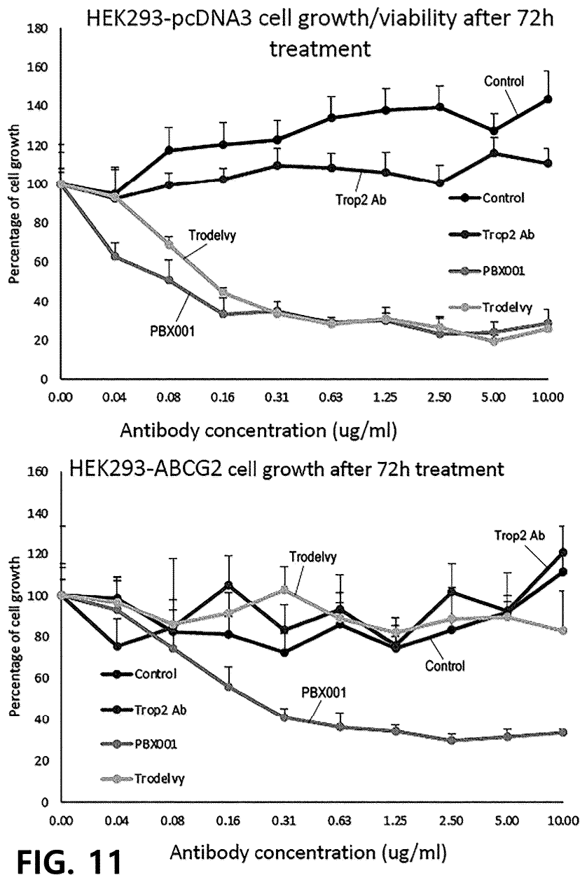

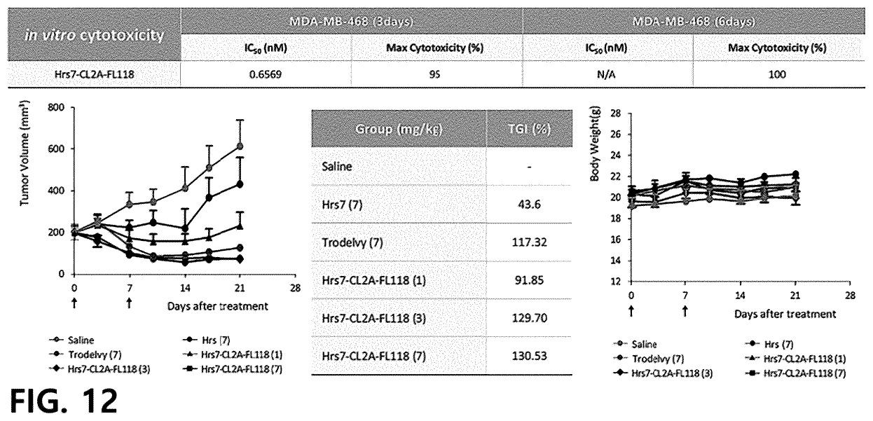

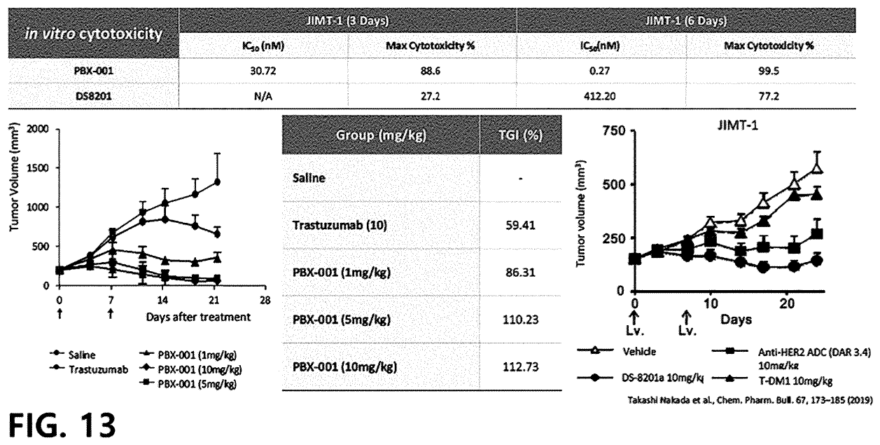

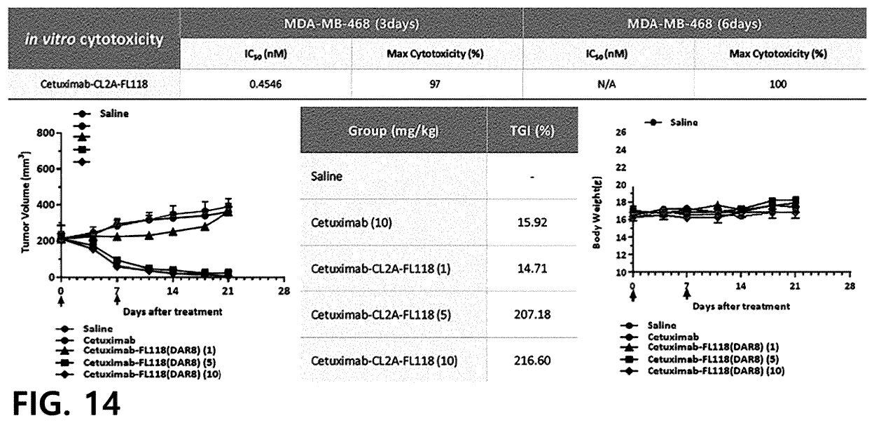

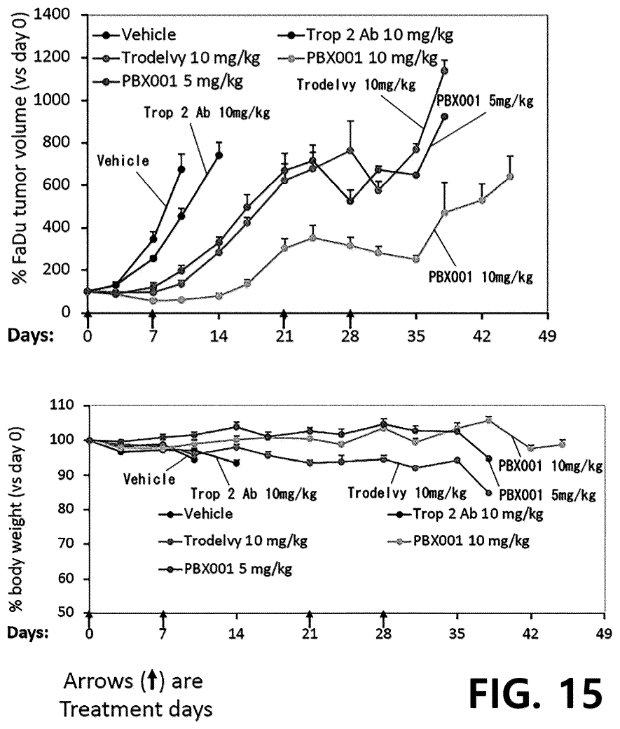

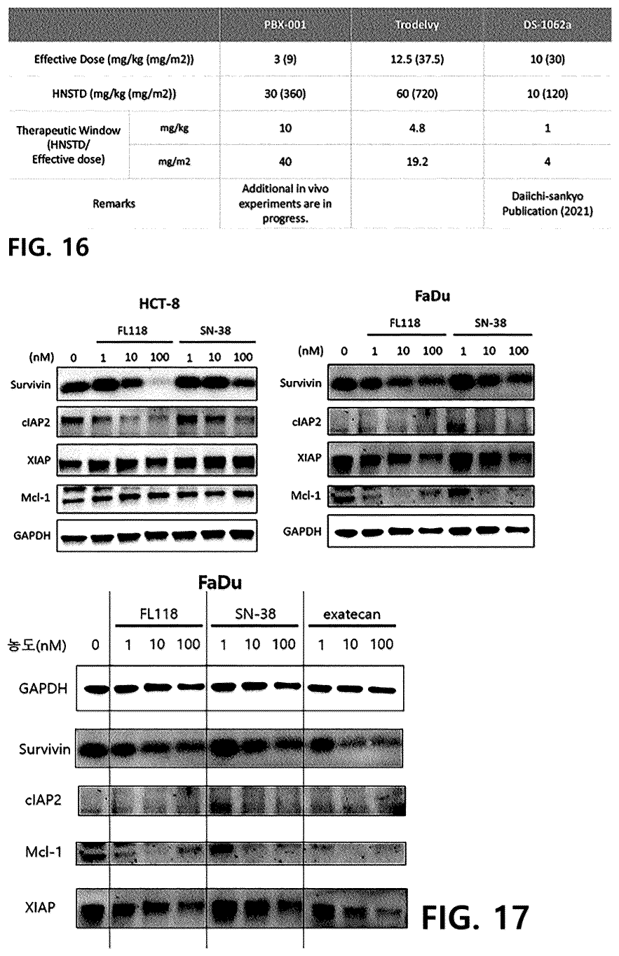

In addition, as shown in FIGS. 8 to 16, the antigen binding assay, stability according to pH, serum stability, efficacy in drug-resistant cells expressing an ABCG2 transporter protein, tumor growth inhibition (TGI) efficacy, excellent anticancer effect, and preclinical safety profile of PBX-001 were confirmed. From this, a conceptual diagram illustrating the MoA of an ADC, including a drug-linker conjugate consisting of the combination of a camptothecin-based drug and an acid-sensitive linker, is shown in FIG. 6.

### Preparation Example 9: Preparation of Anti-HER2 Antibody-CL2A Linker-Exatecan/Dxd Immunoconjugate (DAR 8)

2 mg/mL of anti-HER2 antibodies (trastuzumab) in a reaction buffer (20 mM histidine, 150 mM NaCl, pH 6.0) were reduced with 825 μM TCEP for 2 hours at 25° C., and a thiol site required for reaction was created from a disulfide of the antibody. After reduction, TCEP was removed using a spin desalting column (PD-10).

A solution of DMSO and the compounds in Preparation Example 6 and Preparation Example 7 (5 mM stock in DMSO) was added to the reduced antibody solution with a final DMSO concentration of 10% at 12 eq relative to the antibody. The reaction mixture was mixed properly, and the reaction vial was left at 25° C. for 1 hour.

After conjugation, the reaction mixture was passed through a spin desalting column (PD-10) to remove an unreacted compound and separate only the ADC. Subsequently, the product was sterilized and filtrated through a 0.2-μm PVDF disposable filter. The resulting immunoconjugate was characterized and 0.07 mM PS80 and 20 mM trehalose dehydrate were added.

HIC and MS were used to determine the DAR of the immunoconjugate of Preparation Example 3, and an average MS-DAR value was 7.14, and an HIC-DAR value was 8.00.

### Example 1: Synthesis of Dual Payload-ADC (Trastuzumab-FL118/MMAE)

A trastuzumab-FL118/MMAE dual-drug ADC was prepared by the following method.

After buffer exchange with a reaction buffer (150 mM NaCl, 50 mM histidine, pH 6.0) using a PD-10 desalting column, 27.5 μM of the prepared trastuzumab antibodies were treated with 825 μM tris(2-carboxyethyl)phosphine (TCEP) at 25° C. for 2 hours to create a thiol site required for reaction from a disulfide of the antibody.

Afterward, excess TCEP was removed using a PD-10 desalting column, and a first conjugation reaction was carried out by allowing a 61.9 μM mc-vc-PAB-MMAE and 13.8 μM reduced trastuzumab to react in a 10% DMSO-containing reaction buffer at 4° C. for 1 hour.

After the first reaction, a second reaction with CL2A-FL118 was carried out by two methods: the first method was to carry out the second reaction after removing residual mc-vc-PAB-MMAE using a PD-10 desalting column, and the second method was to add CL2A-FL118 to the first reaction solution without the removal step.

In the first method, the second reaction was carried out using 13.8 μM reduced trastuzumab and 82.5 μM CL2A-FL118, and in the second method, 12.1 μM reduced trastuzumab and 72.4 μM CL2A-FL118 were allowed to react, and the other reaction conditions were the same as those of the first reaction.

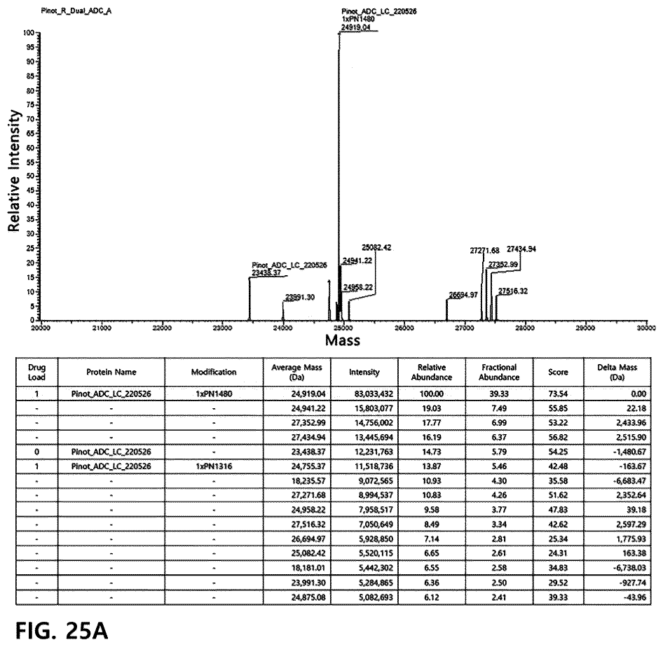

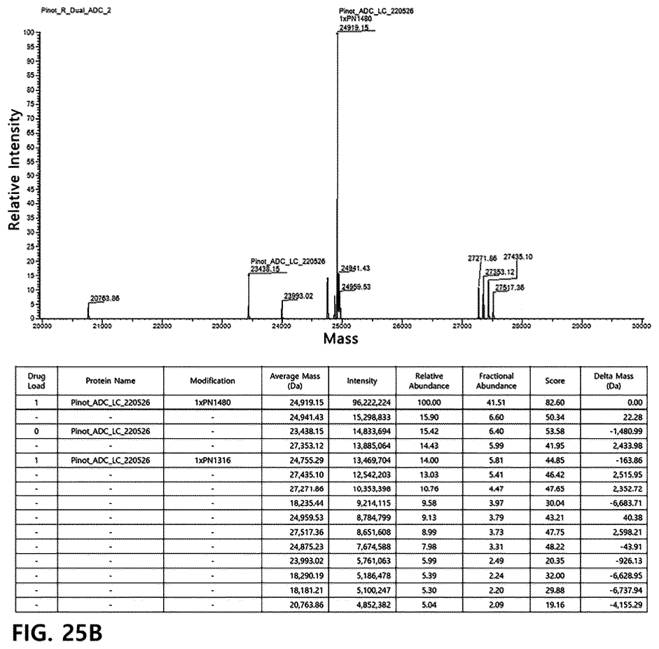

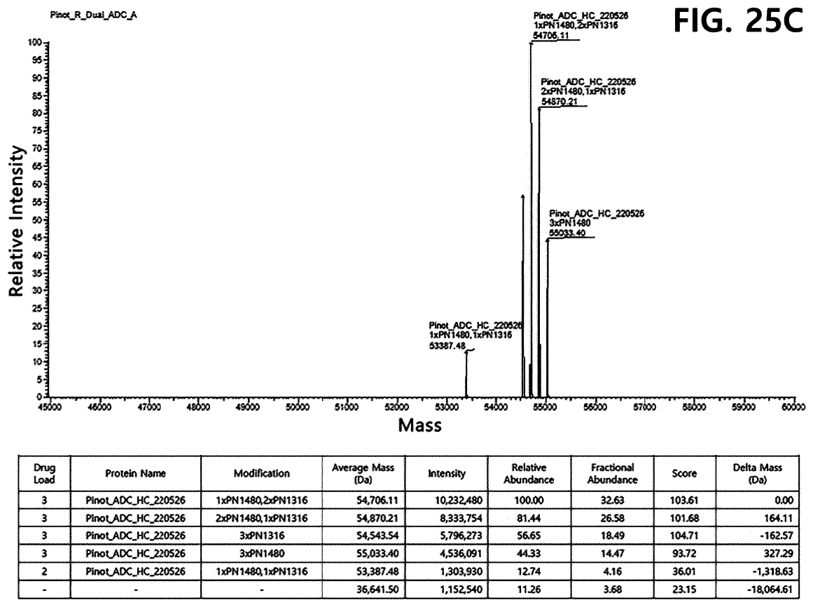

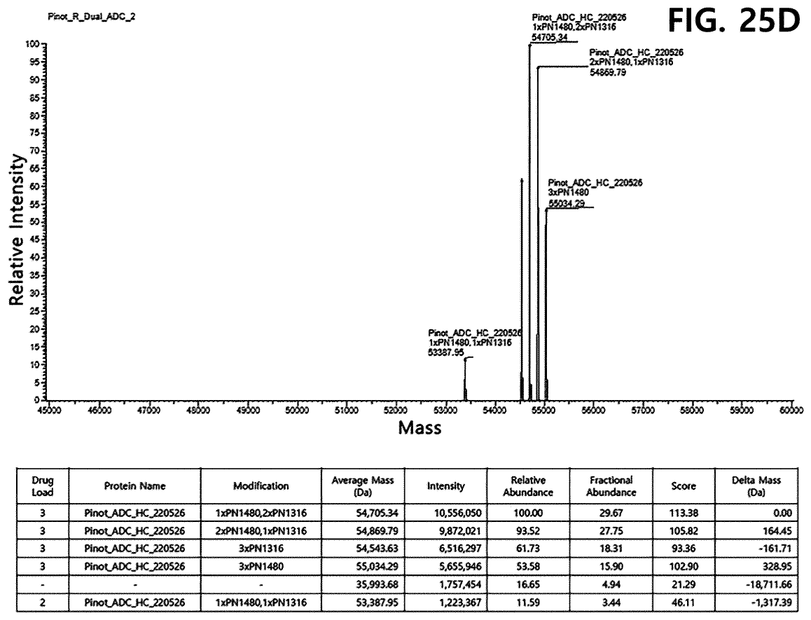

Then, the ADC was purified using SEC, confirming that it was purified as a monomer without aggregation. It was confirmed through SDS-PAGE that the drug was conjugated through the band shifts of a light chain and a heavy chain. Afterward, the DAR of the ADC and the composition ratio of two drugs were analyzed through LC-MS (FIG. 25).

The DAR was calculated from the LC-MS data as follows.

$$FL 118 DAR = 2 * ( average number of FL 118 binding to light chain + average number of FL 118 binding to heavy chain )$$

$$MMAEDAR = 2 * ( average number of MMAE binding to light chain + average number of MMAE binding to heavy chain )$$

$$Total DAR = MMAEDAR + FL 118 DAR$$

TABLE 1

| | When ADC is prepared | When ADC is prepared |
|---|---|---|
| | without removal of | with removal of |
| | excess mc-vc-PAB-MMAE | excess mc-vc-PAB-MMAE |
| FL118 DAR | 4.32 | 4.41 |
| MMAE DAR | 3.36 | 3.27 |
| Total DAR | 7.68 | 7.69 |

From this, it can be seen that there is a preference for the MMAE conjugation site among eight endogenous cysteine residues in the antibody, and that DAR for MMAE is 4 or less.

### Example 2: Preparation of Product 1 (when Payload 1 and Payload 2 are Different)

### A. Steps 1 and 2

According to the method disclosed in an Example of Korean Patent Publication No. 10-2349925, an ADC with DAR 8 was prepared by using the combination of a monoclonal Ab with an IgG structure, i.e., trastuzumab, and a CL2A/FL118 linker-payload having maleimide at an end as a linker-payload. An ADC with a structure of DAR 8 was confirmed using HIC, SEC, and mass spectrometry.

### B. Step 3

See FIG. 38 showing Scheme 2. Gram-Scale Synthesis of Trastuzumab-AJICAP-MMAE (5)

To functionalize lysine 248 of the trastuzumab-CL2A-FL118 ADC with DAR 8 obtained above (A), two Val-Cit-PAB-MMAEs were introduced at lysine 248 using the method described in Scheme 2 and Experimental Section (ACS Omega (2019) Vol. 4, pp. 20564-20570).

Through HIC and SEC analyses, it was confirmed that aggregation occurred at less than 5%, and using mass spectrometry, it was confirmed that an ADC with a structure in which eight FL118s and two MMAEs were introduced was produced.

### Example 3: Preparation of Product 1 (when Payload 1 and Payload 2 are the Same)

### A. Steps 1 and 2

According to the method described in an Example of Korean Patent Publication No. 10-2349925, an ADC with DAR 8 was prepared by using the combination of a monoclonal Ab with an IgG structure, i.e., trastuzumab, and a CL2A/FL118 linker-payload having maleimide at an end as a linker-payload. An ADC with a structure of DAR 8 was confirmed using HIC, SEC, and mass spectrometry.

### B. Step 3

To functionalize the lysine 248 of the trastuzumab-CL2A-FL118 ADC with DAR 8 obtained above (A), two additional CL2A/FL118s were further introduced at the lysine 248 by the method described in Scheme 2 and Experimental Section (ACS Omega (2019) Vol. 4, pp. 20564-20570) except that CL2A/FL118 disclosed in Korean Patent Publication No. 10-2349925 was used as a linker-payload, instead of maleimide-caproyl amide-Val-Cit-PABC-MMAE.

Through HIC and SEC analyses, it was confirmed that aggregation occurred at less than 5%, and using mass spectrometry, it was confirmed that an ADC with a structure in which ten FL118s were introduced was produced.

### Example 4: Preparation of Product 2 (when Payload 1 and Payload 2 are Different)

### A. Steps 1 and 2

An ADC with DAR 4 was prepared using the combination of a monoclonal Ab with an IgG structure, i.e., trastuzumab, and a CL2A/FL118 linker-payload having maleimide at an end as a linker-payload. The detailed experimental method is described in an Example of PCT/KR2021/009204, and an ADC with a structure of DAR 4 was confirmed using HIC, SEC, and mass spectrometry.

### B. Step 3

To functionalize the lysine 248 of the trastuzumab-CL2A-FL118 ADC with DAR 4 obtained above (A), two Val-Cit-PAB-MMAEs were introduced at the lysine 248 using the method described in Scheme 2 and Experimental Section (ACS Omega (2019) Vol. 4, pp. 20564-20570). Through HIC and SEC analyses, it was confirmed that aggregation occurred at less than 5%, and using mass spectrometry, it was confirmed that an ADC with a structure in which four FL118s and two MMAEs were introduced was produced.

### Example 5: Preparation of Product 2 (when Payload 1 and Payload 2 are the Same)

### A. Steps 1 and 2

An ADC with DAR 4 was prepared using the combination of a monoclonal Ab with an IgG structure, i.e., trastuzumab, and a CL2A/FL118 linker-payload having maleimide at an end as a linker-payload. The detailed experimental method is described in an Example of PCT/KR2021/009204, and an ADC with a structure of DAR 4 was confirmed using HIC, SEC, and mass spectrometry.

### B. Step 3

To functionalize the lysine 248 of the trastuzumab-CL2A-FL118 ADC with DAR 4 obtained above (A), but, two additional CL2A/FL118s were introduced at the lysine 248 by the method described in Scheme 2 and Experimental Section (ACS Omega (2019) Vol. 4, pp. 20564-20570), except that CL2A/FL118 disclosed in Korean Patent Publication No. 10-2349925 was used as a linker-payload, instead of maleimide-caproyl amide-Val-Cit-PABC-MMAE. Through HIC and SEC analyses, it was confirmed that aggregation occurred at less than 5%, and using mass spectrometry, it was confirmed that an ADC with a structure in which six FL118s were introduced was produced.

### Comparative Example 1: Method of Preparing THIOMAB-MMAE (DAR 2)

After buffer exchange with a reduction buffer (150 mM NaCl, 50 mM histidine pH 6.0) using a PD-10 desalting column, 27.5 μM THIOMAB (trastuzumab) antibodies were treated with 825 μM TCEP at 25° C. for 2 hours to create a thiol site.

Subsequently, residual TCEP was removed using a PD-10 column, and 13.8 μM THIOMAB was treated with 275 μM dehydroascorbic acid such that interchain cysteine residues that would not be used in conjugation were oxidized to disulfide bonds.

After oxidation, excess dehydroascorbic acid was removed using a PD-10 column, and conjugation was carried out by reacting 68.8 μM mc-vc-PAB-MMAE linker-payload and 13.8 μM THIOMAB, which had been prepared in the previous step, in a reaction buffer (25 mM histidine pH 6.0) containing 10% DMSO at 25° C. for 1 hour.

After conjugation, the excess linker-payload was removed using PD-10, and the conjugation of MMAE was confirmed through the heavy chain band shift of SDS-PAGE and HIC.

### Example 6: Method of Preparing Dual-Drug ADC Using THIOMAB (THIOMAB-MMAE/25-6, DAR 2+6)

An ADC consisting of one trastuzumab antibody, two MMAEs (vc linker) and six PBX-7016s (GGFG linker) (trastuzumab-MMAE(2)-25-6(6)), that is, an ADC with a microtubule inhibitor and a Top1 inhibitor as dual payloads, was prepared as follows.

MMAE was first conjugated in the same manner as in the method of preparing THIOMAB-MMAE (DAR 2) of Comparative Example 1.

After the first conjugation reaction, buffer exchange was performed with a reduction buffer using a PD-10 column. Subsequently, 27.5 μM antibodies were treated with 825 μM TCEP at 25° C. for 2 hours to perform reduction, and a thiol site required for the second conjugation reaction was created from a disulfide of the antibody.

The excess TCEP was removed again using a PD-10 column, and the second conjugation reaction was performed by reacting 165 μM 25-6 linker-payload (Chemical Formula 5 and Preparation Example 5) and 13.8 μM reduced THIOMAB-MMAE in a reaction buffer containing 10% DMSO at 25° C. for 1 hour.

After the conjugation reaction, excess linker-payload was removed using a PD-10 column, the degree of ADC aggregation was confirmed using SEC, and the conjugation of the linker-payload was confirmed through the band shifts of a light chain and a heavy chain through SDS-PAGE.

### Example 7: Method of Preparing Dual-Drug ADC Using THIOMAB (THIOMAB-Veliparib-25-6 DAR 4+4)

An ADC consisting of one trastuzumab antibody, four veliparibs (vc linker), and four PBX-7016s (GGFG linker) (trastuzumab-veliparib(4)-25-6(4)), that is, an ADC with a PAPR inhibitor and a Top1 inhibitor as dual payloads was prepared as follows.

Veliparib was first conjugated in the same manner as the method of preparing THIOMAB-MMAE (DAR 2) in Comparative Example 1, except that veliparib was used instead of MMAE.

After the first conjugation reaction, buffer exchange was performed with a reduction buffer using a PD-10 column. Subsequently, 27.5 μM antibodies were treated with 247.5 μM TCEP at 25° C. for 2 hours to perform reduction, and a thiol site required for the second conjugation reaction was created from a disulfide of the antibody.

Excess TCEP was removed again using a PD-10 column, and the second conjugation reaction was performed by reacting 82.5 μM 25-6 linker-payload and 13.8 μM reduced THIOMAB-veliparib in a reaction buffer containing 10% DMSO at 25° C. for 1 hour.

After the conjugation reaction, the excess linker-payload was removed using a PD-10 column, the degree of ADC aggregation was confirmed using SEC, and the conjugation of the linker-payload was confirmed through the band shifts of a light chain and a heavy chain through SDS-PAGE.

### Comparative Example 2: Method of Preparing Trastuzumab-25-6 (DAR 6)

After buffer exchange with a reduction buffer using a PD-10 desalting column, a thiol site required for reaction was created from a disulfide bond of an antibody by treating 27.5 μM of trastuzumab antibodies, which had been prepared, with 825 μM TCEP at 25° C. for 2 hours.

Subsequently, excess TCEP was removed again using a PD-10 desalting column, and conjugation was performed by reacting 165 μM 25-6 linker-payload (Chemical Formula 5 and Preparation Example 5) and 13.8 μM reduced trastuzumab in a reaction buffer containing 10% DMSO at 25° C. for 1 hour.

After conjugation, excess linker-payload was removed using a PD-10 column, the degree of ADC aggregation was confirmed using SEC, and the conjugation of the linker-payload was confirmed through the band shifts of a light chain and a heavy chain through SDS-PAGE.

### Comparative Example 3: Method of Preparing Trastuzumab-25-6 (DAR 4)

After buffer exchange with a reduction buffer using a PD-10 desalting column, a thiol site required for reaction was created from a disulfide bond of an antibody by treating 27.5 μM of trastuzumab antibodies, which had been prepared, with 247.5 μM TCEP at 25° C. for 2 hours.

Subsequently, the excess TCEP was removed again using a PD-10 desalting column, and conjugation was performed by reacting 82.5 μM 25-6 linker-payload (Chemical Formula 5 and Preparation Example 5) and 13.8 μM reduced trastuzumab in a reaction buffer containing 10% DMSO at 25° C. for 1 hour.

After conjugation, the excess linker-payload was removed using a PD-10 column, the degree of ADC aggregation was confirmed using SEC, and the conjugation of the linker-payload was confirmed through the band shifts of a light chain and a heavy chain through SDS-PAGE.

### Experimental Example 1. Confirmation of Activity to Inhibit the Expression of Cancer-Associated Survival Genes DDX5, p-DDX5, Survivin, Mcl-1, XIAP and cIAP2 Through Western Blotting

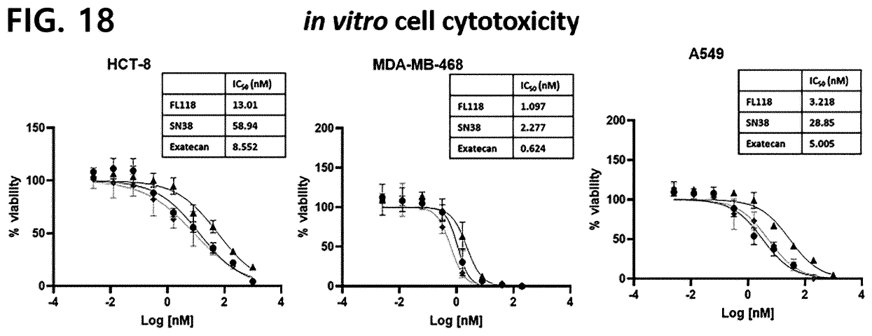

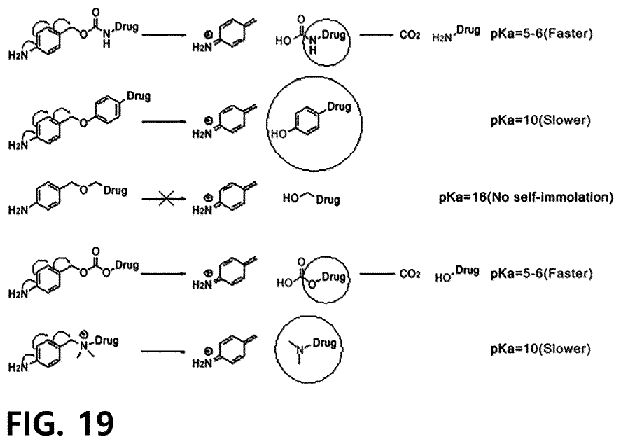

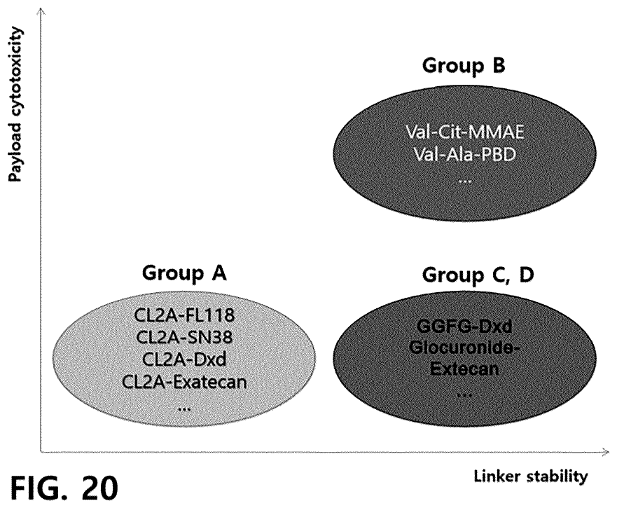

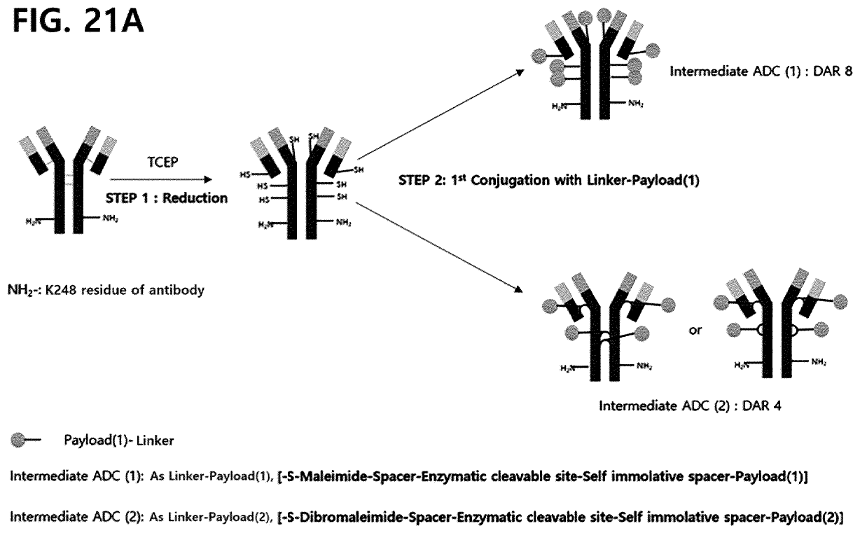

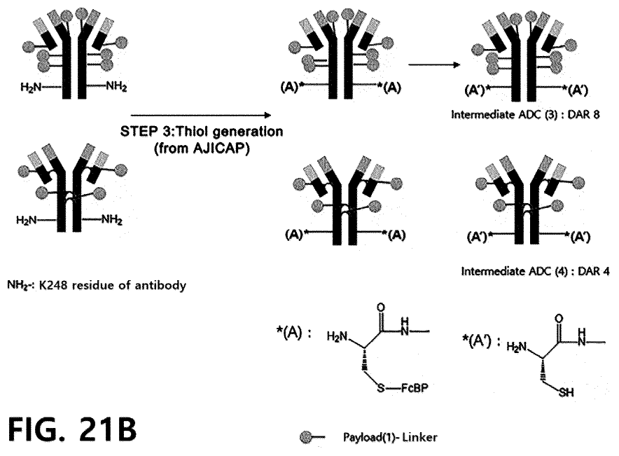

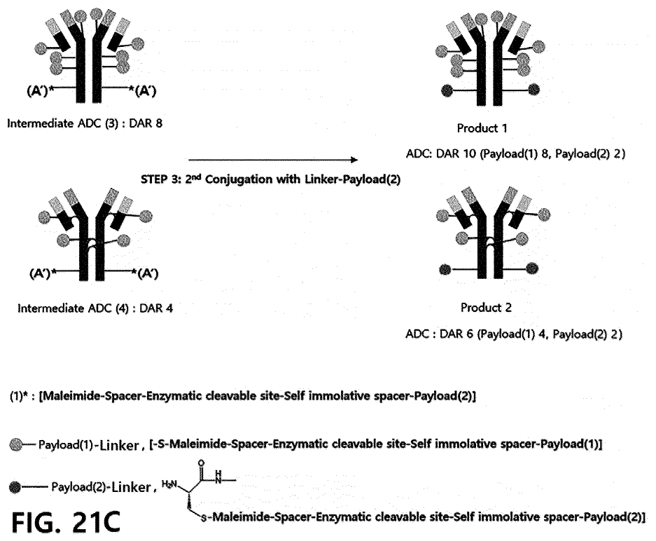

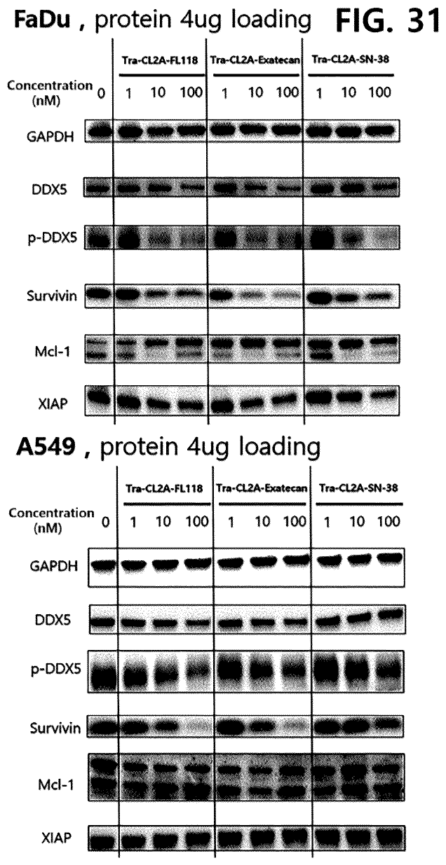

For FL118, exatecan, SN-38, Dxd, PBX-7011, PBX-7014, PBX-7016, Tra-CL2A-FL118, Tra-CL2A-SN-38, and Tra-CL2A-exatecan (Preparation Example 6), an anti-apoptotic protein expression inhibition analysis was performed as follows (FIGS. 17, 26, and 31).

Tra-CL2A-FL118 and Tra-CL2A-SN-38 were prepared in the same manner as in Preparation Example 9, except that the FL118 drug and SN-38 were used instead of exatecan, respectively.

### (1) Protein Extraction

200,000 cells/well of a FaDu cell line not expressing ABCG2 or an A549 cell line overexpressing ABCG2 were seeded in a 6-well plate and cultured (37° C., 5% CO₂). After 24 hours, the wells were respectively treated with drugs (FL118, exatecan, SN-38, Dxd, PBX-7011, PBX-7014, PBX-7016, Tra-CL2A-FL118, Tra-CL2A-SN-38, and Tra-CL2A-exatecan) at concentrations of 0 nM, 10 nM, and 100 nM, and incubated for 24 hours (37° C., 5% CO₂). The wells were treated with 100 μL of RIPA buffer containing a protease inhibitor cocktail. The plate was placed on ice, and incubated on an orbital shaker for 2 hours. The RIPA buffer containing the lysed cells was transferred to an EP tube and centrifuged (16,000 rcf, 20 min, 4° C.). Afterward, the supernatant was transferred to a new EP tube. The protein concentration was confirmed through a protein assay.

### (2) Protein Separation Through Electrophoresis

A protein sample and a 4×SDS-PAGE loading buffer were mixed in a ratio of 3:1, boiled at 95° C. for 10 minutes, and then cooled. The samples were loaded into the wells of a gel so that the amount of protein was the same. Electrophoresis was run on the gel at 60V.

(3) Transfer of Protein from Gel to Membrane

Seven sheets of activated filter paper, a PVDF membrane, the gel, and seven sheets of another filter paper were sequentially placed in cassettes, the lid was closed, the cassette was placed in the Trans-Blot® Turbo™ Transfer System, and then the protocol was run.

### (4) Antibody Incubation

The transferred membrane was immersed in a blocking buffer and incubated (RT, 1 h). Then, it was incubated in a primary antibody solution (4° C., overnight). The membrane was rinsed with a TBST buffer for 3 minutes (repeated three times). The membrane was incubated in an HRP-conjugated secondary antibody solution (RT, 1 h). It was rinsed with a TBST buffer for 3 minutes (repeated three times).

### (5) Imaging and Result Analysis

The membrane was immersed in an ECL substrate for about 3 to 5 minutes, and then signals were confirmed using the ChemiDoc™ MP Imaging System. The light emitted by oxidization of the luminol of ECL by HRP bound to the secondary antibody was detected and displayed as an image. Since the thickness of the band is proportional to the amount of protein, the amount of protein may be compared through band thickness.

As shown in FIGS. 17, 26, and 31, how the expression level of the protein was changed by the treatment of drugs (FL118 drug, SN-38 drug, exatecan drug, PBX-7011, PBX-7014, PBX-7016, Tra-CL2A-FL118, Tra-CL2A-SN-38, and Tra-CL2A-exatecan) was confirmed. GAPDH is an enzyme involved in glycolysis, which is an essential metabolic process in cells. Since it is a gene that is always expressed in cells and its expression level does not change much, it is an indicator of whether equal amounts of protein samples were loaded onto a gel.

It was confirmed that various camptothecin-based compounds degrade DDX5 or phosphorylated DDX5 (p-DDX5), known to be a factor directly involved in the expression of anti-apoptotic proteins. In addition, the expression of anti-apoptotic proteins, such as survivin, Mcl-1, cIAP2, and XIAP, was also partially inhibited, confirming that various camptothecin-based compounds have the dual mechanism of action that not only inhibits topoisomerase-1 but also acts as an anti-apoptotic protein inhibitor.

### Review: Evaluation of PBX-7011, PBX-7014, and PBX-7016

As a result of confirming the degree of inhibiting the expression of anti-apoptotic proteins in the FaDu cell line through Western blotting, it was shown that the higher the concentrations of PBX-7011, PBX-7014, and PBX-7016, the lower the expression of survivin, cIAP2, Mcl-1, and XIAP, and compared to the existing FL118, SN-38, and exatecan drugs, PBX-7011, PBX-7014, and PBX-7016 exhibited similar efficacy to the FL118 and exatecan drugs, but better efficacy than SN-38.

It was confirmed that PBX-7011, PBX-7014, and PBX-7016 exhibit the dual inhibitory action of not only inhibiting topoisomerase-1 but also degrading DDX5, and thus have a heterogeneous characteristic of DDX5 degradation and a subsequent anticancer effect.

Specifically, as a result of treating two cell lines (FaDu and A549) with certain concentrations (0, 10, and 100 nM) of drugs, and confirming the expression level of each protein (DDX, survivin, Mcl-1, and XIAP) through Western blotting, the DDX5 degradation by FL118 was superior to other substances, followed by 7011, 7016, and 7014 in this order.

This was similar not only for DDX5, but also for anti-apoptotic proteins downstream thereof.

In short, PBX-7014 and PBX-7016 showed a dual MoA that does not only inhibit topoisomerase-1, but also acts as a degrader of the oncoprotein DDX5 (p68) that regulates survivin, Mcl-1, and XIAP.

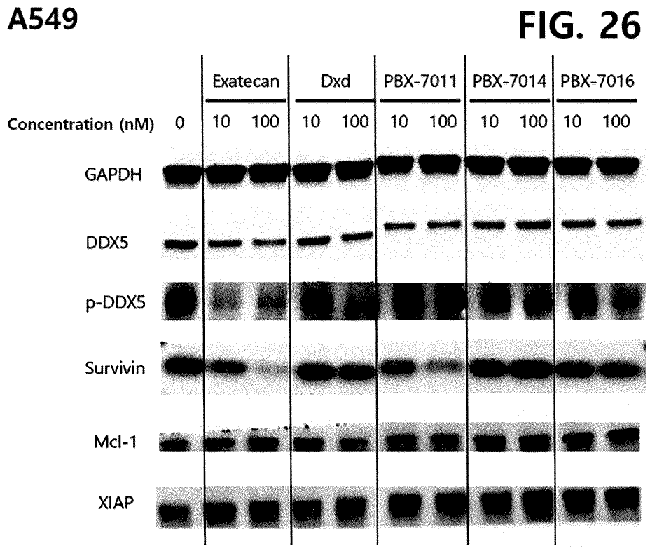

The evaluation results demonstrated that, among PBX-7014 and PBX-7016, PBX-7016 inhibits DDX5 in a concentration-dependent manner, and by observing that PBX-7016 inhibited survivin, Mcl-1, and XIAP, not only did PBX-7016 show the role of FL118 as a DDX5 degrader as well as a topoisomerase I inhibitor, it was confirmed that PBX-7016 works better than PBX-7014 (FIG. 26).

### Experimental Example 2-1. In Vitro Cell Viability Test

Trastuzumab (molecular weight: 148 kDa, purity: 97%), provided from BCD in a solution state stored at −80° C., was dissolved (concentration: 10.09 mg/mL) in a buffer (20 mM histidine, 150 mM NaCl, pH 6.0), and used as a control.

3000 cells/well of a Her2-high cell line (MDA-MB-453) or a HER2 positive breast cancer-derived cell line (SK-BR-3) were seeded in a 96-well plate and incubated (37° C., 5% CO₂). After 24 hours, the cells were treated with 100 L of each drug at nine different concentrations (1/5 serial dilution from 1000 nM). Here, the cells were treated with FL118, exatecan, SN-38, Dxd, PBX-7011, PBX-7014, PBX-7016, PBX-7018, PBX-7020, PBX-7022, PBX-7024, trastuzumab, Tra-CL2A-FL118, and Tra-CL2A-exatecan. After culture (37° C., 5% CO₂) for 3 to 6 days, cell viability was observed. 100 μL of a CellTiter-Glo reagent (using the CellTiter-Glo® Luminescent Cell Viability Assay kit (Promega, G7571)) was added to each well and pipetted. After culture (RT) for 10 minutes, luminescence was measured. Provided that the luminescence value is 100% when the drug concentration is 0, the concentration that exhibits a 50% luminescence value is the IC₅₀ value.

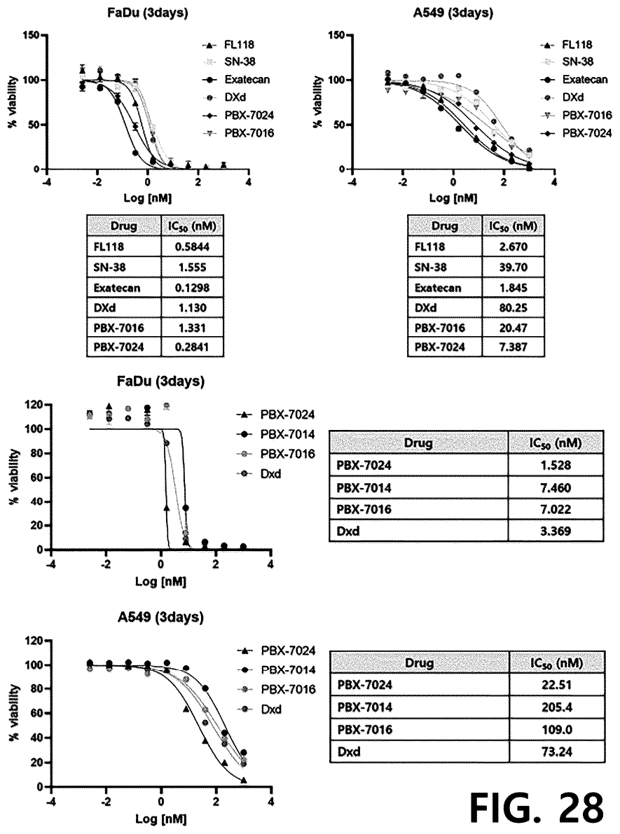

Each of two cell lines (FaDu and A549) was treated with three compounds (PBX-7014, PBX-7016, and PBX-7024) and reference compounds (Dxd, SN-38, exatecan, and FL118), incubated for 3 days, and then cell viability was observed (FIG. 28).

As shown in FIG. 28, PBX-7024 exhibited potent apoptotic efficacy not only in the FaDu cell line not expressing ABCG2, but also in the cancer cell line A549 overexpressing ABCG2. That is, it was confirmed that, compared to Dxd, which is the payload used in the existing Enhertu, PBX-7024 has the same or higher IC₅₀ level.

It can be confirmed that, in the cancer cell line A549 overexpressing ABCG2, as shown in FIG. 28, although the camptothecin compounds, including Dxd, show increased IC₅₀ values, PBX-7016 and PBX-7024 still maintain potent efficacy.

That is, when the novel camptothecin compounds, PBX-7016 and PBX-7024, according to the present invention are used, unlike ADCs using the existing camptothecin compounds, such as SN-38 and DXd, they have the effect of overcoming the resistance mechanism caused by the overexpression of ABCG2.

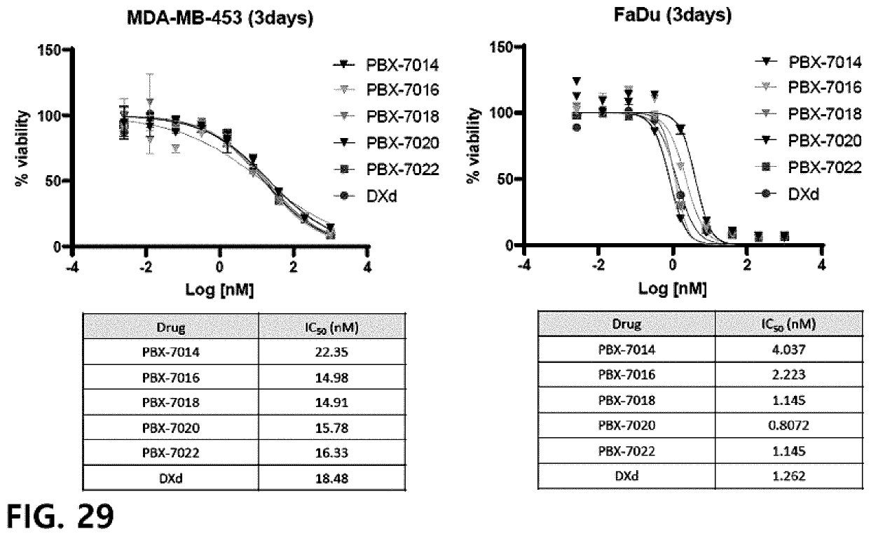

Two cell lines (MDA-MB-453 and FaDu) were treated with five compounds (PBX-7014, PBX-7016, PBX-7018, PBX-7020, and PBX-7022) and one reference compound (Dxd), incubated for 3 days, and then cell viability was observed. The results are shown in FIG. 29.

As shown in FIG. 29, according to the evaluation results, it was confirmed that PBX-7016, PBX-7018, PBX-7020, and PBX-7022 have an IC₅₀ that is equal to or higher than that of Dxd. In the FaDu cell line not expressing ABCG2, PBX-7016, PBX-7018, PBX-7020, and PBX-7022 also have an IC₅₀ that is equal to or higher than that of Dxd.

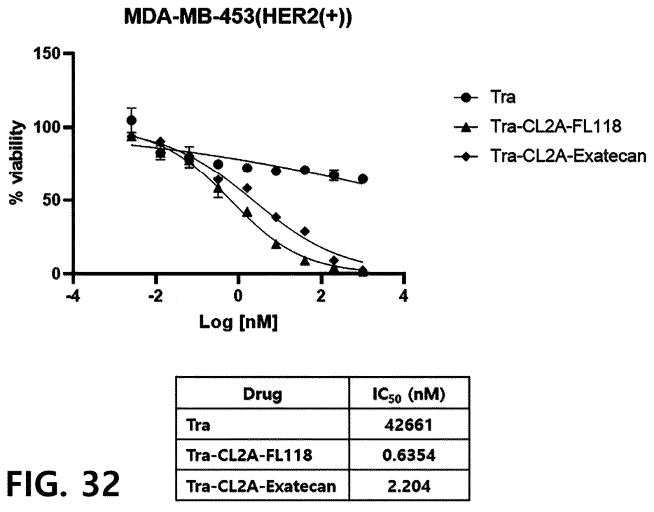

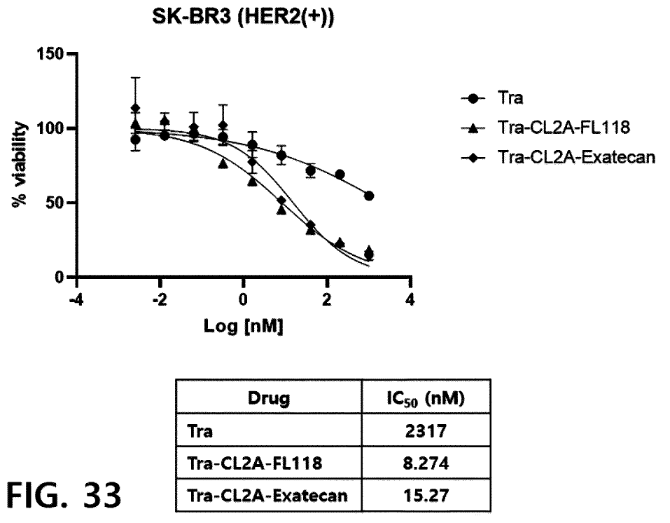

As shown in FIGS. 32 and 33, trastuzumab-CL2A-exatecan (Preparation Example 9) exhibited cytotoxicity in the Her2-high cell line (MDA-MB-453) and the HER2-positive breast cancer-derived cell line (SK-BR-3).

From this, it was confirmed that ADCs in which various camptothecin-based compounds of Chemical Formula 1 or Chemical Formula 2, such as FL118 and exatecan, are included as payloads and are connected by acid-sensitive linkers such as CL2A exhibit good cytotoxicity and act with high efficiency.

### Experimental Example 2-2. New PBX-Series Compounds as Candidate for ADC Payload: In Vitro Cell Viability Test

A cell viability assay was performed on the MDA-MB-453(HER2++) cell line and the FaDu (HER2+) cell line in the same manner as in Experimental Example 2-1.

The two cell lines (MDA-MB-453, FaDu) were treated with five compounds (PBX-7014, PBX-7016, PBX-7018, PBX-7020, and PBX-7022) and one reference compound (Dxd), incubated for 3 days, and then cell viability was observed. The results are shown in FIG. 29.

As shown in FIG. 29, according to the evaluation results, it was confirmed that PBX-7016, PBX-7018, PBX-7020, and PBX-7022 have an IC₅₀ that is equal to or higher than that of Dxd. As shown in FIG. 29, it was confirmed that, compared to the MDA-MB-453 cell line, in the FaDu cell line not expressing ABCG2, PBX-7016, PBX-7018, PBX-7020, and PBX-7022 have the same or higher IC₅₀ than Dxd.

For the ADC using each of Dxd, PBX-7014 and PBX-7016 as a payload, an in vitro cell viability assay was performed on the MDA-MB-453 (HER2++) cell line, the FaDu (HER2+) cell line, and the MDA-MB-468 (HER2−) cell line in the same manner as in Experimental Example 2-2.

Here, a HER2 targeting drug Herceptin (generic name: trastuzumab), the Dxd drug, PBX-7014 (Preparation Example 2), PBX-7016 (Preparation Example 3), and their ADCs Tra-25-4, Tra-25-6 (Preparation Example 5) and Tra-Dxd (Enhertu®), were treated.

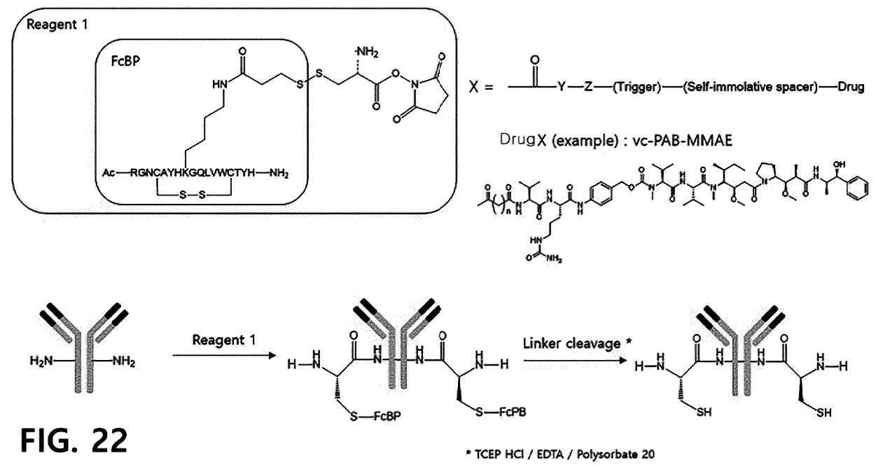

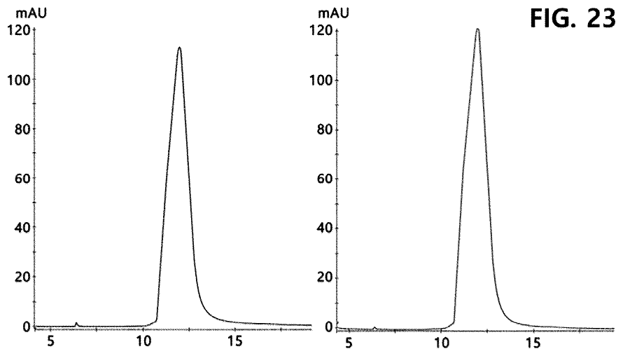

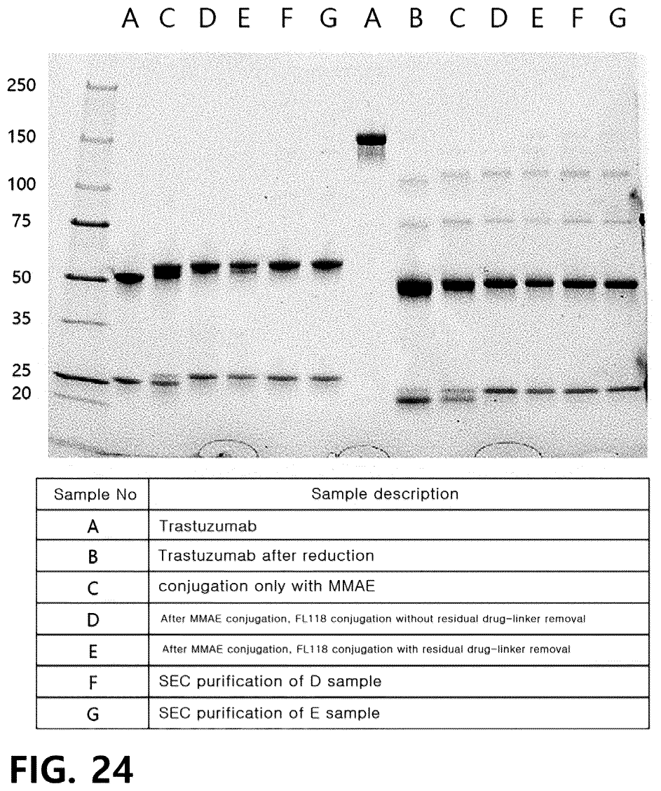

Three cell lines with different Her2 expression levels, MDA-MB-453 (Her2++), FaDu (Her2+), and MDA-MB-468 (Her2−), were treated with Enhertu (Tra-Dxd, DAR 8) as the reference and incubated for 3 days, and then cell viability was observed (FIG. 22).

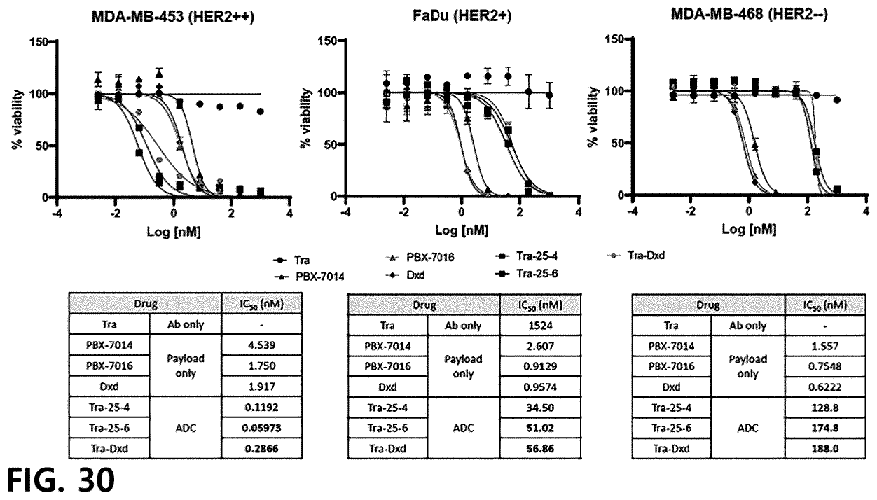

As shown in FIG. 30, it was confirmed that the anti Her2 ADCs with PBX-payloads have excellent target selectivity.

### Review: Evaluation of PBX-7014 and PBX-7016 as ADC Payloads

As shown in FIG. 30, it was confirmed that, in Her2++ MDA-MB-453, the ADC (Tra-25-6) with PBX-7016 as a payload has an IC₅₀ value that is approximately 5 times better than Enhertu (Tra-Dxd), and in the negative cell line MDA-MB-468, the ADC (Tra-25-6) has an equivalent IC₅₀ level, confirming that, when the linker-payload systems of Chemical Formula 4 and Chemical Formula 5 are developed as ADCs, they not only work by distinguishing between positive/negative cell lines, but also show superior efficacy compared to the reference, Enhertu.

### Review: Originality and Excellence of PBX-7016

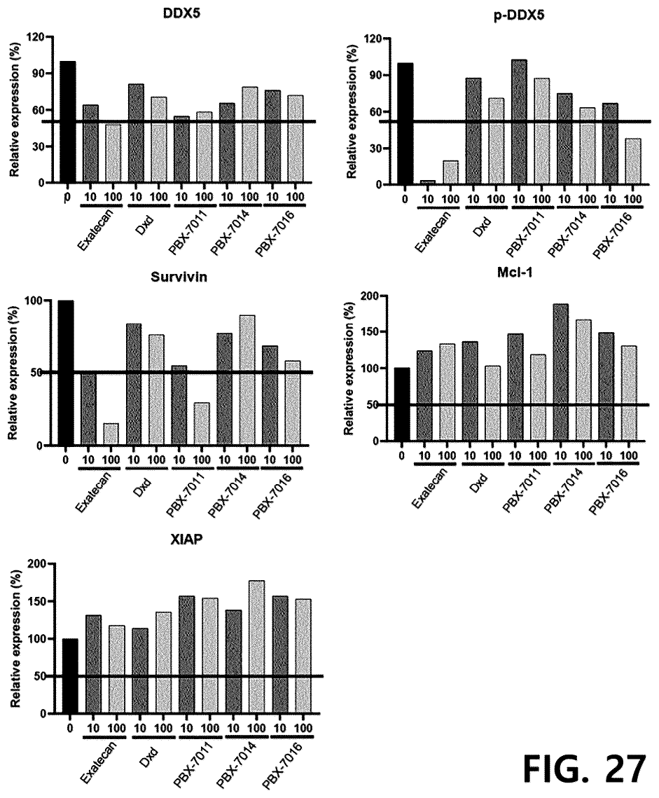

Presented is a new camptothecin-based compound PBX-7016, which is newly designed and synthesized from Chemical Formula 1 according to the present invention, is a compound newly derived from the MD-CPT backbone FL118, and a novel topoisomerase-1 inhibitor that degrades DDX5, like FL118, and acts not only as a topoisomerase I inhibitor but also as a DDX5 degrader (FIGS. 26 and 27).

In addition, when simply comparing cell viability, PBX-7016 has an IC₅₀ value that is 1.5 to 2 times that of Dxd, but surprisingly, when used as an ADC payload, it was confirmed that the PBX-7016-containing ADC has cell viability about 5 times better than the Dxd-containing ADC (Enhertu) (FIG. 30).

### Experimental Example 3. Anti HER2 ADCs with PBX-Payloads: In Vitro Potency and Target Selectivity

For the ADC using each of Dxd, PBX-7014 and PBX-701 as a payload, an in vitro cell viability assay was performed on the MDA-MB-453 (HER2++) cell line, the FaDu (HER2+) cell line, and the MDA-MB-468 (HER2−) cell line in the same manner as in Example 2-2.

Here, a HER2 targeting drug Herceptin (generic name: trastuzumab), the Dxd drug, PBX-7014 (Preparation Example 2), PBX-7016 (Preparation Example 3), and their ADCs Tra-25-4, Tra-25-6 (Preparation Example 5) and Tra-Dxd (Enhertu®), were treated.

Three cell lines with different Her2 expression levels, MDA-MB-453 (Her2++), FaDu (Her2+), and MDA-MB-468 (Her2−), were treated with Enhertu (Tra-Dxd, DAR 8) as the reference and incubated for 3 days, and then cell viability was observed (FIG. 30).

As shown in FIG. 30, it was confirmed that anti Her2 ADCs with PBX-payloads exhibit excellent target selectivity.

### Review: Evaluation of PBX-7014 and PBX-7016 as ADC Payloads

As shown in FIG. 30, it was confirmed that, in the Her2++ MDA-MB-453, the ADC (Tra-25-6) with PBX-7016 as a payload has an IC₅₀ value that is approximately 5 times better than Enhertu (Tra-Dxd), and in the negative cell line MDA-MB-468, the ADC (Tra-25-6) has an equivalent IC₅₀ level, confirming that, when the linker-payload systems of Chemical Formula 4 and Chemical Formula 5 are developed as ADCs, they not only work by distinguishing between positive/negative cell lines, but also show superior efficacy compared to the reference, Enhertu.

### Review: Originality and Excellence of PBX-7016

Presented is a new camptothecin-based compound PBX-7016, which is newly designed and synthesized from Chemical Formula 1 according to the present invention, is a compound newly derived from the MD-CPT backbone FL118, and a novel topoisomerase-1 inhibitor that degrades DDX5, like FL118, and acts not only as a topoisomerase I inhibitor but also as a DDX5 degrader (FIGS. 26 and 27).

In addition, when simply comparing cell viability, PBX-7016 has an IC₅₀ value that is 1.5 to 2 times that of Dxd, but surprisingly, when used as an ADC payload, it was confirmed that the PBX-7016-containing ADC has cell viability about 5 times better than the Dxd-containing ADC (Enhertu) (FIG. 30).

### Experimental Example 4: Anti HER2 ADCs with PBX-Payload: Bystander Effect Study

The bystander effect is one of the factors that significantly affects the efficacy of an ADC drug. To confirm the bystander effect, analysis was performed using FACS under 40 nM ADC treatment conditions.

To verify the efficacy and utility of an antibody-drug conjugate (ADC) in which a payload-linker binds to the trastuzumab antibody targeting the HER2 protein as an ADC drug when using the lead substance of the present invention, PBX-7016, as a payload, an in vitro bystander effect was confirmed through a flow cytometry analysis experiment. In this example, the bystander effect was defined as the induction of cell death by ADC treatment in a HER2-positive cell line, MDA-MB-453, and a drug (payload) released during the cell death exhibits toxicity to a surrounding HER2-negative cell line, leading to cell death.

In Preparation Example 5, to evaluate the in vitro bystander effect of the prepared ADC, the presence of the bystander effect caused by ADC treatment was evaluated under conditions where a HER2 expression-negative cell line and a HER2 expression-positive cell line were mixed and co-cultured at different ratios on a 6-well cell culture plate.

Specifically, after mixing and co-culturing GFP-MDA-MB-468 cells (3×10\hat{ }5 cells) manufactured to express a green fluorescent protein (GFP) in a HER2 expression-negative cell line, MDA-MB-468, and a HER2 expression-positive cell line, i.e., MDA-MB-453 cells (1×10\hat{ }5 cells), in a 3:1 ratio for 24 hours, the cell mixture was treated with each of 40 nM trastuzumab antibody, and five types of ADC samples (Trastuzumab-DXd, Isotype IgG-DXd, Trastuzumab-25-6, Isotype IgG-25-6, and Enhertu) and cultured for 6 days, and then among living cells, the GFP fluorescence and cell count of the HER2 expression-negative cell line, i.e., GFP-MDA-MB-468 cells, were checked through flow cytometry.

In addition, after mixing and co-culturing the HER2 expression-negative cell line, i.e., MDA-MB-468 cells (3×10\hat{ }5 cells), and the HER2 expression-positive cell line, i.e., MDA-MB-453 cells (1×10\hat{ }5 cells) in a 3:1 ratio for 24 hours on a 6-well cell culture plate, the cell mixture was treated with each of 40 nM trastuzumab antibody, and five types of ADC samples (Trastuzumab-DXd, Isotype IgG-DXd, Trastuzumab-25-6, Isotype IgG-25-6, and Enhertu) and cultured for 6 days. Then, after cell staining using an anti-HER2-FITC antibody that was designed to exhibit fluorescence by labeling a fluorescein isothiocyanate (FITC) fluorochrome dye to an antibody that can recognize the HER2 protein, among living cells, the FITC fluorescence and cell count of the HER2 expression-positive cell line, i.e., MDA-MB-453 cells, were checked through flow cytometry.

For flow cytometry, after the cells present in each well of the 6-well cell culture plate were harvested and diluted with a buffer (400 μL), GFP or FITC fluorescence was measured for each sample for the same time (1 min each) and at the same flow rate using FL1 laser.

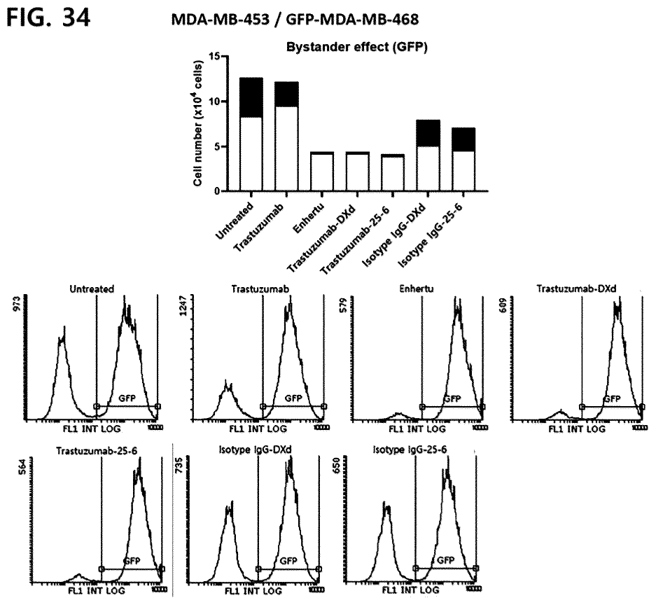

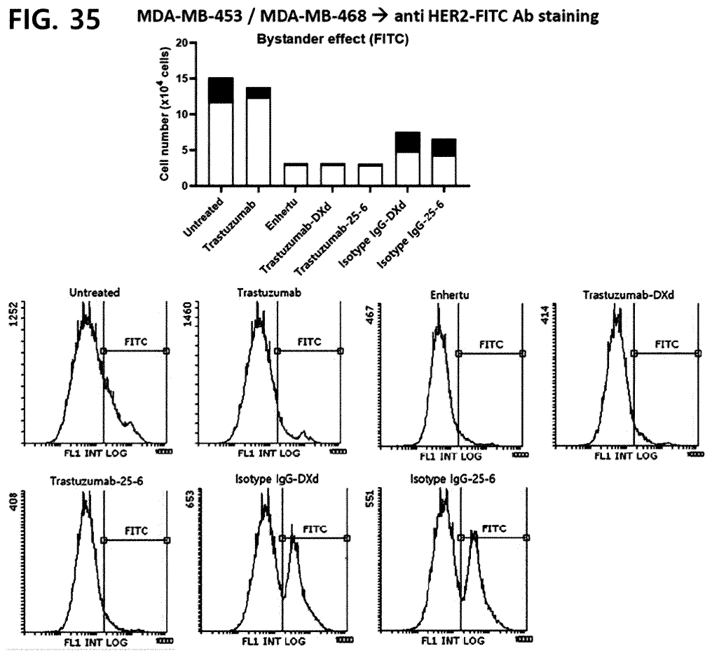

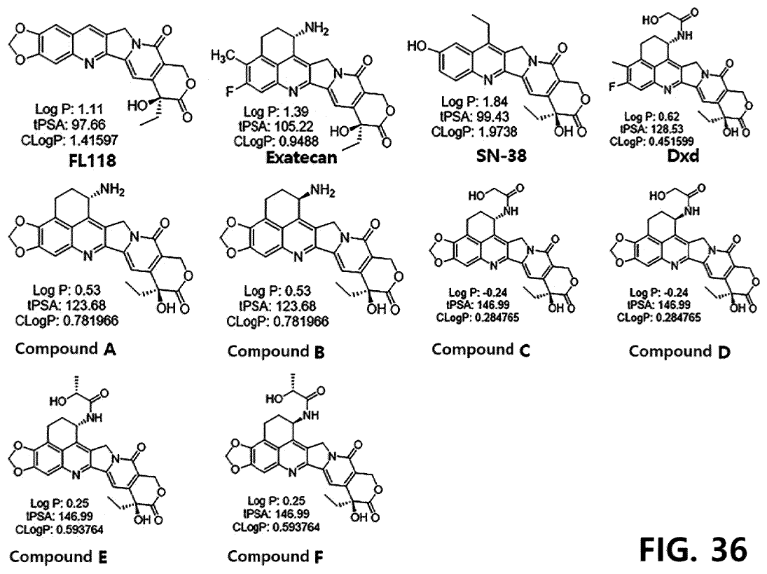

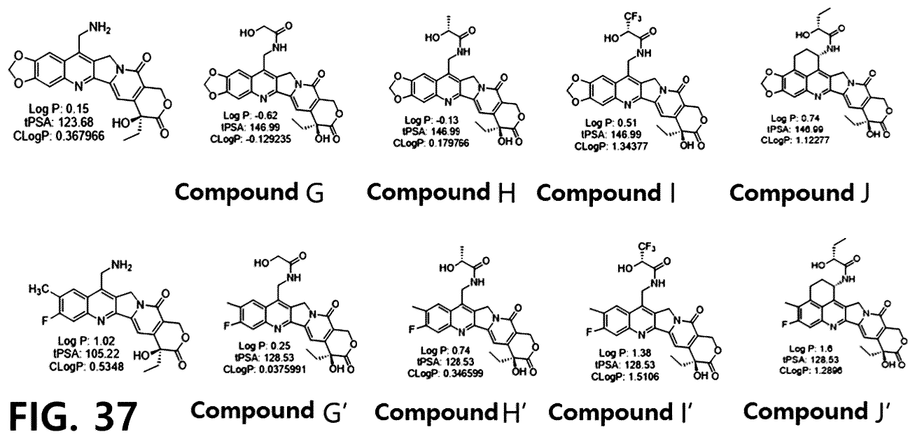

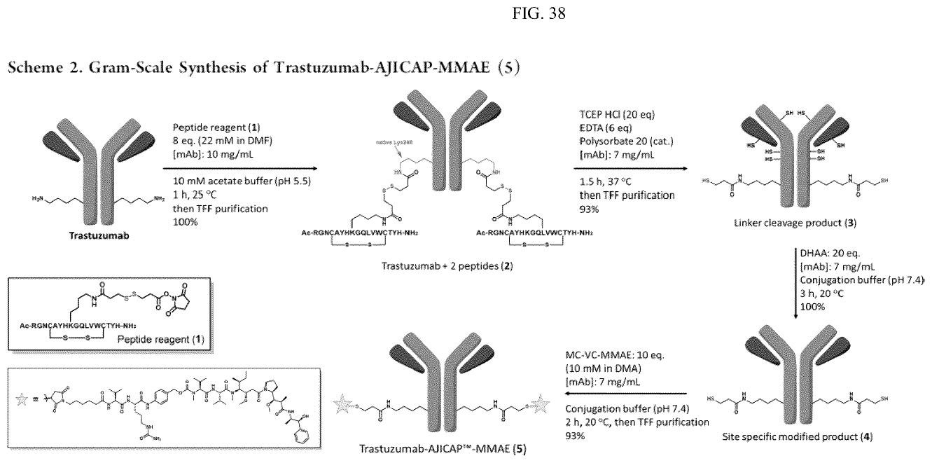

As shown in FIGS. 34 and 35, from the results confirmed through flow cytometry, it was determined that, compared with DXd-based ADCs, Trastuzumab-Dxd and Enhertu, the PBX-7016-based ADC, Trastuzumab-25-6 not only killed HER2 expression-positive cells but also simultaneously killed HER2 expression-negative cells, showing a bystander effect at the same level as the existing Dxd-based Enhertu. On the other hand, it was confirmed that negative control ADCs, Isotype IgG-DXd and Isotype IgG-25-6, do not greatly induce HER2 expression-negative cell death, compared to non-ADC-treated conditions.

In short, an anti HER2 ADC using PBX-7016 exhibited a bystander effect similar to Enhertu.

### Experimental Example 5. In Vitro Cell Viability Test for Dual Payload-ADC Payload

Cell viability assay was performed on the MDA-MB-453 cell line and the MDA-MB-468 cell line in the same manner as in Experimental Example 2-1.

Both MDA-MB-453 and MDA-MB-468 are cell lines that are generally used in cancer research, particularly, breast cancer studies.

MDA-MB-453: This cell line is extracted from a metastatic site of human breast adenocarcinoma. It is known to be estrogen receptor-negative (ER−) and progesterone receptor-negative (PR−), meaning that MDA-MB-453 cells do not express these hormone receptors. MDA-MB-453 cells overexpress human epithelial growth factor receptor 2 (HER2) and exhibit amplification of the ERBB2 gene, indicating HER2 positivity. In addition, the cells are known to have a TP53 mutation. Researchers often use MDA-MB-453 cells to study HER2-positive breast cancer and test new therapeutic approaches that target HER2.

MDA-MB-468: This cell line was established from the pleural effusion of a 51-year-old female with metastatic breast adenocarcinoma. MDA-MB-468 cells are triple-negative breast cancer (TNBC) cells, which lack the expression of the estrogen receptor (ER−), progesterone receptor (PR−), and human epithelial growth factor receptor 2 (HER2−). These cells are also known to have a TP53 mutation. TNBC is generally more aggressive and has a poorer prognosis compared to other subtypes of breast cancer. Researchers utilize MDA-MB-468 cells to investigate TNBC biology and test potential treatments for this subtype.

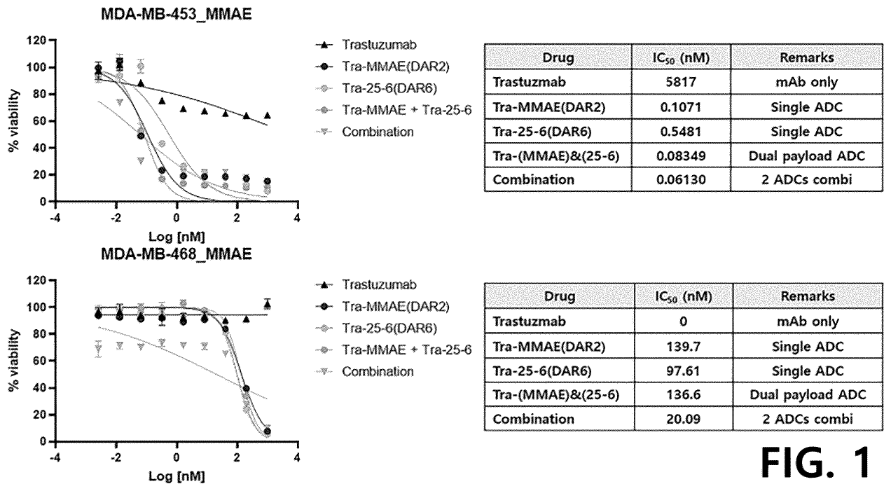

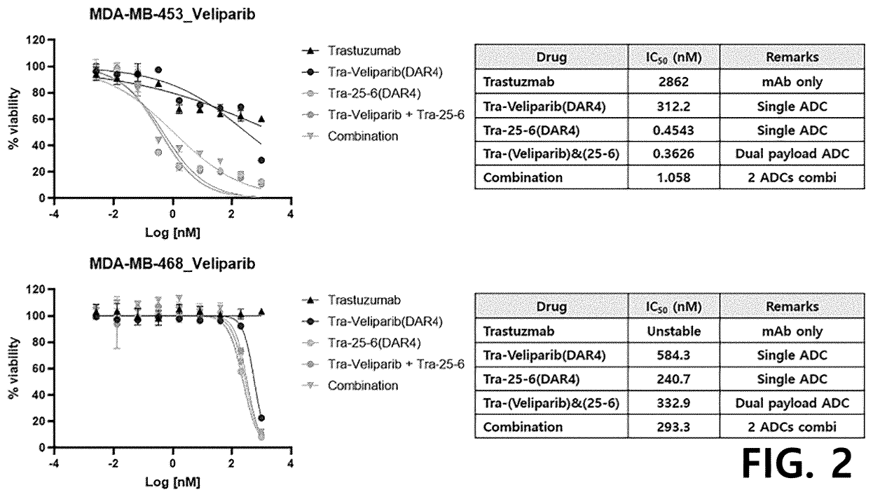

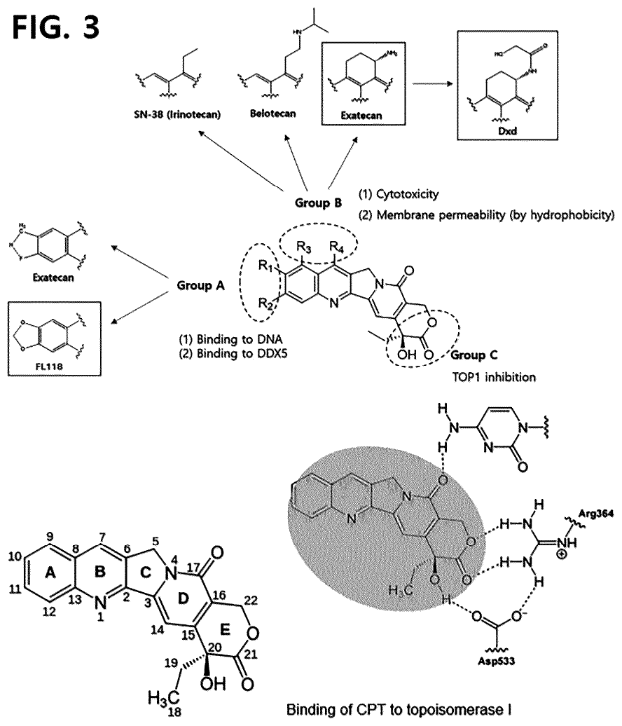

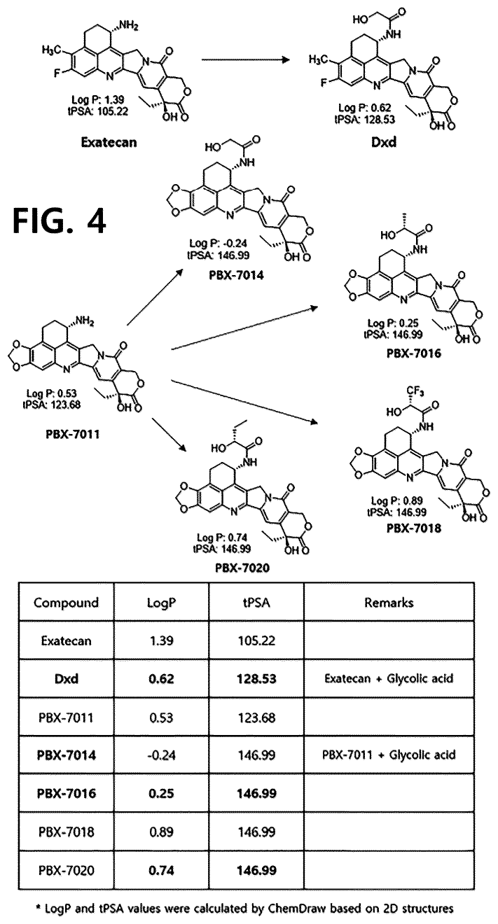

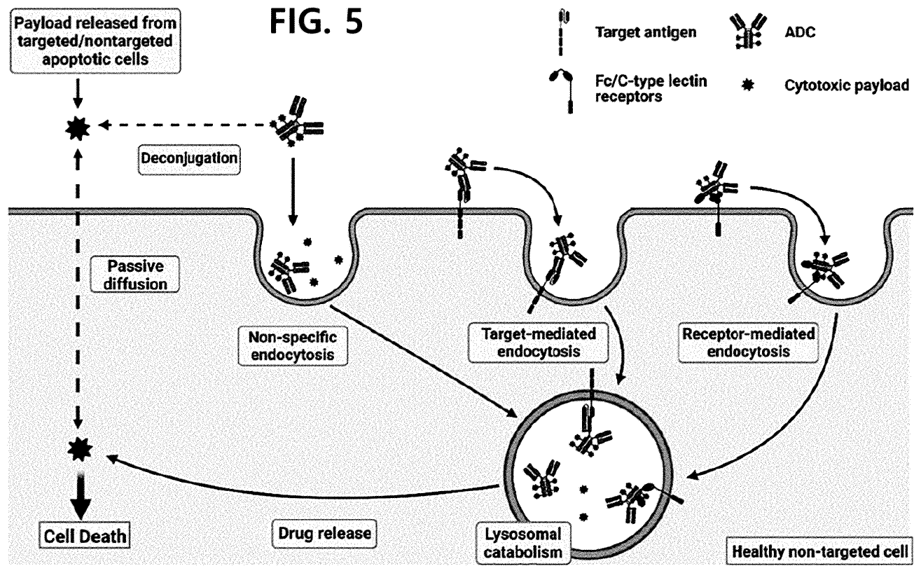

FIGS. 1 and 2 show the evaluation results of dual payload ADCs according to Examples 6 and 7. That is, these are evaluation results of dual payload ADCs, single payload ADCs, and a combination of two single payload ADCs.

The two types of dual payload ADCs synthesized according to Examples 6 and 7, and the single ADC as a reference for the two types of dual payload ADCs are as follows.
(1) Trastuzumab-MMAE(2)-25-6(6): An ADC that has one trastuzumab antibody, two MMAEs (vc linker), and six PBX-7016s (GGFG linker), that is, an ADC with a microtubule inhibitor and a Top1 inhibitor as dual payloads (Example 6)
(2) Trastuzumab-veliparib(4)-25-6(4): An ADC that has one trastuzumab antibody, four veliparibs (vc linkers) and four PBX-7016s (GGFG linkers), that is, an ADC with a PAPR inhibitor and a Top1 inhibitor as dual payloads (Example 7)
Single ADC: a reference for the two types of dual-payload ADCs
Trastuzumab-25-6 (DAR 4) (Comparative Example 3)
Trastuzumab-25-6 (DAR 6) (Comparative Example 2)
Trastuzumab-MMAE (DAR 2) (Comparative Example 1)
Trastuzumab-veliparib (DAR 4)

### Review:

Analysis of the IC₅₀ results of Trastuzumab-MMAE(2)-25-6(6) in Her2 positive cell line MDA-MB-453 and Her2 negative cell line MDA-MB-468

In the Her2 positive cell line MDA-MB-453,
(1) the dual payload ADC exhibited higher potency (lower IC₅₀) than the single payload ADCs, and
(2) the dual-payload ADC exhibited similar levels of potency in an in vitro experiment to a combination of single payload ADCs (Trastuzumab-25-6 (DAR 6)+Trastuzumab-MMAE (DAR 2).

However, in the Her2 negative cell line MDA-MB-468,
(3) the dual payload ADC had a significantly higher IC₅₀ value than a combination of single payload ADCs (in terms of off-target toxicity of ADC), confirming high stability.

### Results of Evaluation of Trastuzumab-Veliparib(4)-25-6(4)

In the Her2 positive cell line MDA-MB-453,
(1) the dual payload ADC exhibited higher potency (lower IC₅₀) than the single payload ADCs, and
(2) the dual-payload ADC exhibited a similar level of potency (lower IC₅₀) in an in vitro experiment to a combination of single payload ADCs (Trastuzumab-veliparib (DAR 4)+Trastuzumab-25-6 (DAR 4)).

However, in the Her2 negative cell line MDA-MB-468,
(3) the dual payload ADC was confirmed to have stability relatively the same or higher than that of a combination of the single payload ADCs (Trastuzumab-veliparib (DAR 4)+Trastuzumab-25-6 (DAR 4)).

Taken together, it was confirmed that (1) a dual payload ADC exhibits superior potency to a single ADC with the same DAR, (2) when compared with a combination of two types of single ADCs with the same DAR, a dual payload ADC exhibits an equivalent level of potency and relatively higher stability (in the case of Trastuzumab-MMAE(2)-25-6(6)), and (3) when compared with a combination of two types of single ADCs with the same DAR, a dual payload ADC exhibits an equivalent level of stability and relatively higher potency (in the case of Trastuzumab-veliparib(4)-25-6(4)).

1. A method of preparing an antibody-drug conjugate (ADC), in which a drug-linker conjugate (A) consisting of the combination of a camptothecin-based drug that degrades DDX5 protein with a DAR=4 or higher and (i) an acid-sensitive linker or (ii) an enzyme-sensitive linker is linked to one antibody, and which is designed to increase the therapeutic index of the ADC or its payload, which is a camptothecin-based drug, and inhibit the non-selective uptake of the camptothecin-based drug and/or ADC, which is released from apoptotic cells, the method comprising:
designing and/or synthesizing an ADC in which two types of drug-linker conjugates are linked such that a drug-linker conjugate (A) consisting of the combination of a camptothecin-based drug that degrades the DDX5 protein with a DAR of 4 or higher, and (i) an acid-sensitive linker or (ii) an enzyme-sensitive linker; and a drug-linker conjugate (B) consisting of the combination of a non-camptothecin-based cytotoxic drug and an enzyme-sensitive linker are linked to one antibody.

2. The method of claim 1, wherein the camptothecin-based drug degrading the DDX5 protein is an active camptothecin derivative represented by Chemical Formula 1 or Chemical Formula 2 below, designed to bind to the DDX5 protein and an E3 ligase, 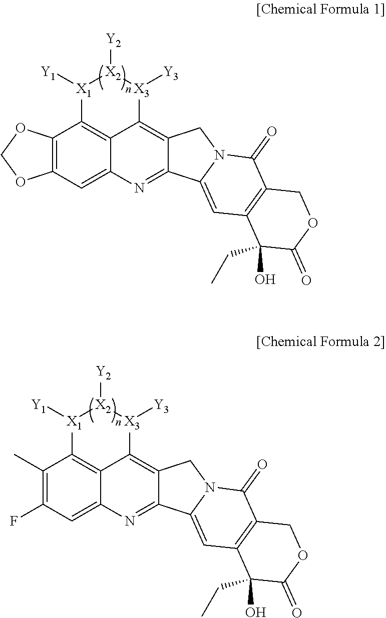
here, X₁ and X₃ are each independently carbon, oxygen, nitrogen, or sulfur, and are the same or different,
X₂ is carbon, oxygen, nitrogen, sulfur, a single bond, or a double bond,
X₁, (X₂)ₙ and X₃ form a 5-, 6- or 7-membered ring (n=0 to 2), and
Y₁, Y₂ and Y₃ are each independently hydrogen, or a functional group containing oxygen, nitrogen, phosphorus or sulfur.

3. The method of claim 1, wherein the additional connection of the drug-linker conjugate (B) consisting of the combination of a non-camptothecin-based cytotoxic drug and an enzyme-sensitive linker inhibits the non-selective uptake of the camptothecin-based drug-containing ADC in non-target cells of the ADC.

4. The method of claim 2, wherein the non-camptothecin-based cytotoxic drug alleviates or inhibit adverse effects of the ADC by regulating the excessive bystander effect of the camptothecin-based drug released from both target/non-target apoptotic cells.

5. The method of claim 2, wherein the non-camptothecin-based cytotoxic drug solves the off-target toxicity problem of the camptothecin-based drug released together from target/non-target apoptotic cells.

6-14. (canceled)

15. An antibody-drug conjugate (ADC) in which two types of drug-linker conjugates are linked to one antibody, comprising
a drug-linker conjugate (A) consisting of a combination of the camptothecin-based drug degrading DDX5 protein with a DAR of 4 or more and (i) an acid-sensitive linker or (ii) an enzyme-sensitive linker; and
a drug-linker conjugate (B-1) consisting of a combination of a non-camptothecin super toxic drug having a DAR of 4 or less and an enzyme-sensitive linker or a drug-linker conjugate (B-2) consisting of a combination of an anti-apoptotic protein inhibitor drug and an enzyme-sensitive linker, which are linked to one antibody.

16. The ADC of claim 15, wherein the camptothecin-based drug is an active camptothecin derivative represented by Chemical Formula 1 or Chemical Formula 2, designed to bind to the DDX5 protein and an E3 ligase, 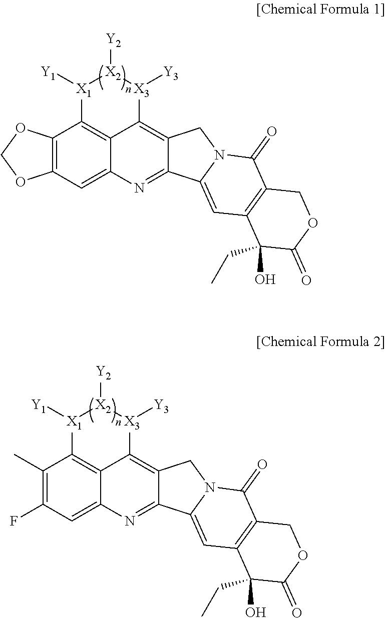
here, X₁ and X₃ are each independently carbon, oxygen, nitrogen, or sulfur, and are the same or different,
X₂ is carbon, oxygen, nitrogen, sulfur, a single bond, or a double bond,
X₁, (X₂)ₙ and X₃ form a 5-, 6- or 7-membered ring (n=0 to 2), and
Y₁, Y₂ and Y₃ are each independently hydrogen, or a functional group containing oxygen, nitrogen, phosphorus or sulfur.

17. The ADC of claim 15, wherein the combination of a non-camptothecin-based super toxic drug or an anti-apoptotic protein inhibitor drug is used as a payload to prevent cells not expressing a target antigen from absorbing the camptothecin-based drug-containing ADC.

18. The ADC of claim 15, wherein the non-camptothecin-based super toxic drug or anti-apoptotic protein inhibitor drug inhibits the bystander effect of a free camptothecin-based drug released from target/non-target apoptotic cells.

19. The ADC of claim 15, wherein the non-camptothecin-based super toxic drug or anti-apoptotic protein inhibitor drug overcomes the off-target toxicity problem of the free camptothecin-based drug released from target/non-target apoptotic cells.

20. The ADC of claim 15, wherein a dose of the ADC is 2 to 10 mg/kg, and preferably, 4 to 10 mg/kg.

21. The ADC of claim 15, wherein the non-camptothecin-based super toxic drug is a tubulin disruptor or a PARP inhibitor.

22. The ADC of claim 15, wherein, after targeting the ADC including the drug-linker conjugate (A) with a combination of [camptothecin-based drug]-[acid-sensitive linker] to cancer cells by an antibody targeting an antigen of the cancer cells,
the acid-sensitive linker is degraded in an acidic environment (pH≤7) around cancer to release at least part of the camptothecin-based drug, and the free-camptothecin-based drug migrates into the cells through the cell membrane, and
optionally, the camptothecin-based drug-linked ADC is internalized within the cells to release the camptothecin-based drug from lysosomes.

23. The ADC of claim 15, wherein the acid-sensitive linker is stable in a neutral environment (pH 7.3 to 7.5) of bloodstream, but hydrolyzed around tumor cells (pH 6.5 to 7.2) or in endosomes (pH 5.0 to 6.5) or lysosomes (pH 4.5 to 5.0) after intracellular internalization, thereby releasing the active drug.

24. The ADC of claim 15, wherein, after being targeted to cancer cells by an antigen-binding site targeting an antigen of the cancer cells, the acid-sensitive linker is degraded in an acidic environment (pH≤7) around cancer to release at least part of the camptothecin-based drug, thereby releasing the drug both inside and outside the cells.

25. The ADC of claim 15, wherein the drug-linker conjugate (A) consisting of a combination of the camptothecin-based drug and of (i) an acid-sensitive linker or (ii) an enzyme-sensitive linker; and the drug-linker conjugate (B-1) consisting of a combination of a non-camptothecin-based super toxic drug and an enzyme-sensitive linker or the drug-linker conjugate (B-2) consisting of a combination of an anti-apoptotic protein inhibitor drug and an enzyme-sensitive linker are each independently connected to a cysteine or lysine residue of the antibody.

26. The ADC of claim 15, wherein the drug-linker conjugate (A) consisting of a combination of the camptothecin-based drug and a (i) an acid-sensitive linker or (ii) an enzyme-sensitive linker; and the drug-linker conjugate (B-1) consisting of a combination of a non-camptothecin-based super toxic drug and an enzyme-sensitive linker or the drug-linker conjugate (B-2) consisting of a combination of an anti-apoptotic protein inhibitor drug and an enzyme-sensitive linker are homogeneously and symmetrically linked to the antibody.

27. The ADC of claim 15, wherein the antibody targets one of human epidermal growth factor receptors (HER/EGFR/ERBB), such as HER2, FolR, PSMA, or Trop-2

28. A pharmaceutical composition for preventing or treating cancer, which comprises the antibody-drug conjugate (ADC) of claim 15, or a pharmaceutically acceptable salt thereof as an active ingredient.

29. The pharmaceutical composition of claim 28, wherein the antibody is trastuzumab, cetuximab, or sacituzumab.
